(19) United States
(12) Patent Application Publication
(10) Pub. No.: US 2024/0237600 A1
(43) Pub. Date: Jul. 18, 2024

(54) ELITE SAFFLOWER EVENT

(71) Applicant: Commonwealth Scientific and Industrial Research Organisation, Acton (AU)

(72) Inventors: Shoko Okada, Coombs (AU); Amratha Menon, Casey (AU); Anu Mathew, Moncrieff (AU); Craig Christopher Wood, Dickson (AU)

(73) Assignee: Commonwealth Scientific and Industrial Research Organisation, Acton (AU)

(21) Appl. No.: 18/565,463

(22) PCT Filed: May 31, 2021
(86) PCT No.: PCT/AU2021/050533

§ 371 Date: Nov. 29, 2023

(51) Int. Cl. A01H 1/00; A01H 5/10; A01H 6/14; C12N 9/02; C12N 9/12

(52) U.S. Cl. CPC A01H 1/104; A01H 5/10; A01H 6/1416; C12N 9/0071; C12N 9/1205

(57) ABSTRACT

The present invention relates to elite transgenic safflower events which produce high levels of oleic acid.

### FIELD OF THE INVENTION

The present invention relates to elite transgenic safflower events which produce high levels of oleic acid.

### BACKGROUND OF THE INVENTION

Plant oils are an important source of dietary fat for humans, representing about 25% of caloric intake in developed countries (Broun et al., 1999). World production of plant oils is at least about 110 million tons per year, of which 86% is used for human consumption. Almost all of these oils are obtained from oilseed crops such as soy bean, canola, safflower, sunflower, cottonseed and groundnut, or plantation trees such as palm, olive and coconut (Gunstone, 2001: Oil World Annual, 2004). The growing scientific understanding and community recognition of the impact of the individual fatty acid components of food oils on various aspects of human health is motivating the development of modified vegetable oils that have improved nutritional value while retaining the required functionality for various food applications. These modifications require knowledge about the metabolic pathways for plant fatty acid synthesis and genes encoding the enzymes for these pathways (Liu et al., 2002: Thelen and Ohlrogge, 2002).

Considerable attention is being given to the nutritional impact of various fats and oils, in particular the influence of the constituents of fats and oils on cardiovascular disease, cancer and various inflammatory conditions. High levels of cholesterol and saturated fatty acids in the diet are thought to increase the risk of heart disease and this has led to nutritional advice to reduce the consumption of cholesterol-rich saturated animal fats in favour of cholesterol-free unsaturated plant oils (Liu et al., 2002).

While dietary intake of cholesterol present in animal fats can significantly increase the levels of total cholesterol in the blood, it has also been found that the fatty acids that comprise the fats and oils can themselves have significant effects on blood serum cholesterol levels. Of particular interest is the effect of dietary fatty acids on the undesirable low density lipoprotein (LDL) and desirable high density lipoprotein (HDL) forms of cholesterol in the blood. In general, saturated fatty acids, particularly myristic acid (14:0) and palmitic acid (16:0), the principal saturates present in plant oils, have the undesirable property of raising serum LDL-cholesterol levels and consequently increasing the risk of cardiovascular disease (Zock et al., 1994; Hu et al., 1997). However, it has become well established that stearic acid (18:0), the other main saturate present in plant oils, does not raise LDL-cholesterol, and may actually lower total cholesterol (Bonanome and Grundy, 1988: Dougherty et al., 1995). Stearic acid is therefore generally considered to be at least neutral with respect to risk of cardiovascular disease (Tholstrup, et al., 1994). On the other hand, unsaturated fatty acids, such as the monounsaturate oleic acid (18:1), have the beneficial property of lowering LDL-cholesterol (Roche and Gibney, 2000), thus reducing the risk of cardiovascular disease.

Oil high in oleic acid also has many industrial uses such as, but not limited to, lubricants often in the form of fatty acid esters, biofuels, raw materials for fatty alcohols, plasticizers, waxes, metal stearates, emulsifiers, personal care products, soaps and detergents, surfactants, pharmaceuticals, metal working additives, raw material for fabric softeners, inks, transparent soaps, PVC stabilizer, alkyd resins, and intermediates for many other types of downstream oleochemical derivatives.

Oil processors and food manufacturers have traditionally relied on hydrogenation to reduce the level of unsaturated fatty acids in oils, thereby increasing their oxidative stability in frying applications and also providing solid fats for use in margarine and shortenings. Hydrogenation is a chemical process that reduces the degree of unsaturation of oils by converting carbon-carbon double bonds into carbon-carbon single bonds. Complete hydrogenation produces a fully saturated fat. However, the process of partial hydrogenation results in increased levels of both saturated fatty acids and monounsaturated fatty acids. Some of the monounsaturates formed during partial hydrogenation are in the trans isomer form (such as elaidic acid, a trans isomer of oleic acid) rather than the naturally occurring cis isomer (Sebedio et al., 1994; Fernandez. San Juan, 1995). In contrast to cis-unsaturated fatty acids, trans-fatty acids are now known to be as potent as palmitic acid in raising serum LDL cholesterol levels (Mensink and Katan, 1990; Noakes and Clifton, 1998) and lowering serum HDL cholesterol (Zock and Katan, 1992), and thus contribute to increased risk of cardiovascular disease (Ascherio and Willett, 1997). As a result of increased awareness of the anti-nutritional effects of trans-fatty acids, there is now a growing trend away from the use of hydrogenated oils in the food industry, in favour of fats and oils that are both nutritionally beneficial and can provide the required functionality without hydrogenation, in particular those that are rich in either oleic acid where liquid oils are required or stearic acid where a solid or semi-solid fat is preferred.

There is a need for further lipids and oils with high oleic acid content and reliable sources thereof.

### SUMMARY OF THE INVENTION

The present inventors have identified elite lines of safflower which can be used for commercial scale production of oil comprising high levels of oleic acid.

In an aspect, the present invention provides a safflower plant cell comprising
(a) a polynucleotide which comprises a sequence of nucleotides provided as SEQ ID NO:13 and a polynucleotide which comprises a sequence of nucleotides provided as SEQ ID NO: 14,
(b) a polynucleotide which comprises a sequence of nucleotides provided as SEQ ID NO:21 and a polynucleotide which comprises a sequence of nucleotides provided as SEQ ID NO:22,
(c) a T-DNA molecule, wherein the T-DNA molecule is inserted into a region of a chromosome of the cell, wherein the region has a nucleotide sequence provided as SEQ ID NO:11,
(d) a T-DNA molecule, wherein the T-DNA molecule is inserted into a region of a chromosome of the cell, wherein the region has a nucleotide sequence provided as SEQ ID NO:18, or
(e) any combination thereof.

In an embodiment, the safflower plant cell comprises (a) and (c), for example both (a) and (c) apply in the case of GOR73226. In an embodiment, the safflower plant cell comprises (b) and (d), for example both (b) and (d) apply in the case of GOR73240. In an embodiment, the safflower plant cell comprises (a) to (d), for example where the safflower line GOR73226 is crossed with the line GOR73240 so that the safflower cell comprises both T-DNAs.

In a preferred embodiment, the cell has (a) or (b) but not both. The (a) or (b) can be present in a heterozygous state or preferably in a homozygous state in the safflower genome. In the homozygous embodiments, the safflower cell lacks either SEQ ID NO:11 (in the case of (a)) or lacks SEQ ID NO:18 (in the case of (b)) as contiguous sequences due to the T-DNA insertion.

In another aspect, the present invention provides a safflower plant cell comprising one or more of
(a) a polynucleotide which comprises a sequence of nucleotides provided as SEQ ID NO:7,
(b) a polynucleotide which comprises a sequence of nucleotides provided as nucleotides 296 to 8640 of SEQ ID NO:7,
(c) a polynucleotide which comprises a sequence of nucleotides provided as SEQ ID NO:33,
(d) a polynucleotide which comprises a sequence of nucleotides provided as SEQ ID NO:34, or
(e) a polynucleotide having at least 95% identity with the full length of nucleotides 296 to 8640 of SEQ ID NO:7.

In a preferred embodiment, all of (a), (b) and (e) apply. In an embodiment, (a), (b), (c) and (e) apply, for example in GOR73226. In an embodiment, (a), (b), (d) and (e) apply, for example in GOR73240. In a preferred embodiment, the cell has a single T-DNA insertion.

In an embodiment, the polynucleotide(s) and/or the T-DNA(s) are stably integrated into the genome of the cell, preferably into the nuclear genome of the cell. In an embodiment, the safflower plant cell, or a plant or plant part of the invention, is homozygous for the polynucleotide(s) and/or the T-DNA(s). Alternatively, the safflower cell, plant or plant part is heterozygous for the polynucleotide(s) and/or the T-DNA(s), for example in the case of F1 seed.

In a further aspect, the present invention provides a cell of safflower line GOR73226 or GOR73240.

In an embodiment, oleic acid comprises at least 90% by weight of the total fatty acids in a safflower plant cell of the invention. In an embodiment, oleic acid comprises between 90% to 95% by weight of the total fatty acids in a safflower plant cell of the invention. In an embodiment, oleic acid comprises between 91% to 93% by weight of the total fatty acids in a safflower plant cell of the invention. In an embodiment, oleic acid comprises between 91.5% to 92.5% by weight of the total fatty acids in a safflower plant cell of the invention.

In an embodiment, at least 95% by weight of the lipid in a plant cell of the invention is triacylglycerol (TAG).

In an embodiment, palmitic acid comprises less than 2.7% by weight of the total fatty acids in a safflower plant cell of the invention. In an embodiment, palmitic acid comprises between 2.5% and 2.7% by weight of the total fatty acids in a safflower plant cell of the invention.

In an embodiment, linoleic acid comprises less than 1.7% by weight of the total fatty acids in a safflower plant cell of the invention. In an embodiment, linoleic acid comprises between 1.2% and 1.6% by weight of the total fatty acids in a safflower plant cell of the invention.

In an embodiment, α-linolenic acid comprises less than 0.2% by weight of the total fatty acids in a safflower plant cell of the invention. In an embodiment, α-linolenic acid (ALA) comprises less than 0.1%, or about 0.1%, by weight of the total fatty acids in a safflower plant cell of the invention. In an embodiment, the total fatty acids in a safflower plant cell of the invention do not comprise α-linolenic acid, and/or the α-linolenic acid is undetectable.

In an embodiment, a safflower plant cell of the invention comprises a hygromycin phosphotransferase polypeptide, and a polynucleotide encoding the polypeptide, such as a hygromycin phosphotransferase polypeptide comprising a sequence of amino acids as provided in SEQ ID NO:8.

In an embodiment, a safflower plant cell of the invention, where the cell is a safflower seed cell, has reduced CtFAD2-2 protein activity and reduced CtFATB-3 protein activity relative to a corresponding safflower cell lacking the polynucleotide(s) and/or the T-DNA(s). In an embodiment of the safflower seed cell, the level of CtFAD2-2 protein activity is reduced by between 90% and 99% relative to a corresponding safflower cell lacking the polynucleotide(s) and/or the T-DNA(s), preferably reduced by between 95% and 99% or between 96% and 99%. In an embodiment, the level of CtFATB-3 protein activity is reduced by between 50% and 95% relative to a corresponding safflower cell lacking the polynucleotide(s) and/or the T-DNA(s), preferably reduced by between 60% and 95%, or between 75% and 95%. In an embodiment, a safflower plant cell of the invention has some CtFATB-3 protein activity and some CtFATB-3 protein activity. In an embodiment of the safflower cell, which is a cell other than a seed cell, the CtFAD2-2 and CtFATB-3 protein activities are not significantly reduced relative to a corresponding safflower cell lacking the polynucleotide(s) and/or the T-DNA(s). This phenotype is associated with the seed-specific property of the promoter driving the polynucleotide, for example in GOR73226 and GOR73240. In an embodiment, the safflower cell other than a seed cell nevertheless produces the Hph polypeptide.

In an embodiment, a safflower plant cell of the invention comprises an ol allele of the CtFAD2-1 gene or an oll allele of the CIFAD2-1 gene, or both alleles. In a preferred embodiment, the ol allele or the oll allele of the CIFAD2-1 gene is present in the homozygous state. In a preferred embodiment, the allele is an ol allele.

In an embodiment, a safflower plant cell of the invention is a seed cell, either in a developing seed or in a mature seed. In an embodiment, a safflower plant cell of the invention is in a seed of a safflower plant growing in a field or in a harvested seed.

In another aspect, the present invention provides a safflower seed comprising a cell comprising
(a) a polynucleotide which comprises a sequence of nucleotides provided as SEQ ID NO:13 and a polynucleotide which comprises a sequence of nucleotides provided as SEQ ID NO: 14,
(b) a polynucleotide which comprises a sequence of nucleotides provided as SEQ ID NO:21 and a polynucleotide which comprises a sequence of nucleotides provided as SEQ ID NO:22,
(c) a T-DNA molecule, wherein the T-DNA molecule is inserted into a region of a chromosome of the cell, wherein the region has a nucleotide sequence provided as SEQ ID NO:11,
(d) a T-DNA molecule, wherein the T-DNA molecule is inserted into a region of a chromosome of the cell, wherein the region has a nucleotide sequence provided as SEQ ID NO:18, or
(e) any combination thereof.

In another aspect, the present invention provides a safflower seed comprising a cell comprising comprising one or more of
(a) a polynucleotide which comprises a sequence of nucleotides provided as SEQ ID NO:7,
(b) a polynucleotide which comprises a sequence of nucleotides provided as nucleotides 296 to 8640 of SEQ ID NO: 7,
(c) a polynucleotide which comprises a sequence of nucleotides provided as SEQ ID NO:33,
(d) a polynucleotide which comprises a sequence of nucleotides provided as SEQ ID NO:34, or
(e) a polynucleotide having at least 95% identity with the full length of nucleotides 296 to 8640 of SEQ ID NO:7.

In a further aspect, the present invention provides a seed of safflower line GOR73226 or GOR73240.

In another aspect, the present invention provides a safflower seed comprising a cell of the invention.

A seed of the invention can have any of the features outlined above in relation to a cell of the invention. For example, in an embodiment oleic acid comprises at least 90% by weight of the total fatty acids in a safflower plant seed of the invention, preferably between 90% and 95%.

In a further aspect, the present invention provides a collection of safflower seeds, wherein at least 95% of the seeds are seeds of the invention.

In another aspect, the present invention provides a safflower plant, or part thereof, comprising a cell of the invention. In an embodiment, the plant or part thereof comprises
(a) a polynucleotide which comprises a sequence of nucleotides provided as SEQ ID NO:13 and a polynucleotide which comprises a sequence of nucleotides provided as SEQ ID NO: 14,
(b) a polynucleotide which comprises a sequence of nucleotides provided as SEQ ID NO:21 and a polynucleotide which comprises a sequence of nucleotides provided as SEQ ID NO:22,
(c) a T-DNA molecule, wherein the T-DNA molecule is inserted into a region of a chromosome of the cell, wherein the region has a nucleotide sequence provided as SEQ ID NO:11,
(d) a T-DNA molecule, wherein the T-DNA molecule is inserted into a region of a chromosome of the cell, wherein the region has a nucleotide sequence provided as SEQ ID NO:18, or
(e) any combination thereof.

In another aspect, the present invention provides a safflower plant, or part thereof, comprising a cell comprising comprising one or more of
(a) a polynucleotide which comprises a sequence of nucleotides provided as SEQ ID NO:7,
(b) a polynucleotide which comprises a sequence of nucleotides provided as nucleotides 296 to 8640 of SEQ ID NO:7,
(c) a polynucleotide which comprises a sequence of nucleotides provided as SEQ ID NO:33,
(d) a polynucleotide which comprises a sequence of nucleotides provided as SEQ ID NO:34, or
(e) a polynucleotide having at least 95% identity with the full length of nucleotides 296 to 8640 of SEQ ID NO: 7.

In a further aspect, the present invention provides a safflower line GOR73226 or GOR73240, or a part thereof.

In another aspect, the present invention provides a safflower plant, or part thereof, comprising a cell of the invention.

A plant of the invention can have any of the features outlined above in relation to a cell of the invention. For example, in an embodiment oleic acid comprises at least 90% by weight of the total fatty acids in a safflower plant part, such as a seed, of the invention, preferably between 90% and 95%.

In an embodiment, the plant comprises cells of the invention. In an embodiment, the part is a seed. In an embodiment, the plant comprises, or is capable of producing, a seed of the invention.

The present inventors have found that plants of the invention produce seed with consistently high levels of oleic acid. In an embodiment, the level of oleic acid in the seed is consistent over multiple generations, such as nine generations. In an embodiment, the level of oleic acid in the seed is at least 90%, between 90% to 95%, between 91% to 93% or between 91.5% to 92.5% by weight of the total fatty acids over multiple generations, such as nine generations. In an embodiment, the level of oleic acid by weight of the total fatty acid content varies less than 5%, less than 4%, less than 2% or less than 1% over multiple generations, such as nine generations.

The present inventors have also found that plants of the invention produce seed with an oil content that is not significantly reduced compared to the oil content of seed of a corresponding safflower plant lacking the polynucleotide(s) and T-DNA(s) of the invention, grown under the same conditions, or is preferably increased in oil content relative to the nontransgenic seed. In an embodiment, the oil content is increased by 1-4% on an absolute basis relative to the nontransgenic seed, for example from 31% to at least 32% or 35%. In an embodiment, the oil content of the seed of the invention is consistent over multiple generations, such as nine generations. In an embodiment, the oil content is at least 33% or at least 34% by weight, preferably between 33% and 37% by weight or between 34% and 37% by weight. In an embodiment, the oil content varies less than 5%, less than 4%, less than 2% or less than 1% over multiple generations, such as nine generations.

In an embodiment, a safflower plant, or part thereof, of the invention is produced by growing the seed of the invention.

In an embodiment, the polynucleotide(s) and/or the T-DNA(s) are stably integrated into the genome of the plant, or part thereof (such as a seed), for at least nine generations.

In an embodiment, one or more or all of the following features are the same as a corresponding plant lacking the polynucleotide(s) and/or the T-DNA(s) grown under the same conditions: seedling vigour, plant height, time to flowering, harvest lodging, seed crude protein content, seed crude fat content, seed ash content and seed carbohydrate content.

In an embodiment, the safflower plant, or part thereof, comprises a transgene other than the polynucleotide(s) and/or the T-DNA(s).

Also provided is pollen of a plant of the invention.

Also provided is an ovule of a plant of the invention.

Also provided is a tissue culture of regenerable cells, wherein the cells are cells of the invention. In an embodiment, the tissue is selected from the group consisting of leaves, pollen, embryos, roots, root tips, pods, flowers, ovules and stems.

In another aspect, the present invention provides a method for producing a safflower plant or seed therefrom, the method comprising:
(a) crossing a first safflower plant of the invention with a second safflower plant to yield progeny safflower seed: and
(b) growing the progeny safflower seed, under plant growth conditions, to yield a progeny safflower plant, and optionally
(c) harvesting seed from the progeny safflower plant.

In an embodiment, the second safflower plant has at least one agronomically desirable trait that is lacking in the first safflower plant. Examples of suitable agronomic traits include, but are not limited to, herbicide resistance, insect resistance, bacterial disease resistance, fungal disease resistance, viral disease resistance, female sterility or male sterility. In an embodiment, the agronomic trait is conferred by a transgene.

In an embodiment, the method further comprises repeating steps (a) and (b) one or more times.

In an embodiment, the harvested seed is a first generation (F1) hybrid safflower seed.

In an embodiment, the second safflower plant is a safflower plant of the invention.

In an embodiment, the second safflower plant does not produce seed of the invention.

In an embodiment, the method further comprises
(d) backcrossing one or more progeny plants from step (b) with plants of the same genotype as the second safflower plant for a sufficient number of times to produce a plant with at least 75%, at least at least 80% or at least 90% of the genotype of the second safflower plant.

Also provided is a safflower plant, or part thereof, produced by a method of the invention.

In a further aspect, the present invention provides a method of identifying a safflower plant, the method comprising analysing DNA obtained from the plant for one or more of the polynucleotide(s) and/or T-DNA molecule(s) defined herein. In a related aspect, the present invention includes a method of determining whether or not a plant, plant part, preferably a seed, is a plant, plant part or seed of the invention, by analysing DNA obtained from the plant, plant part or seed for one or more of the polynucleotide(s) and/or T-DNA molecule(s) defined herein. In an embodiment, a collection of safflower seeds is assayed to determine whether or not a seed of the invention is present in that collection.

In an embodiment, the polynucleotide(s) and/or T-DNA molecule(s) are detected using a technique selected from the group consisting of: restriction fragment length polymorphism analysis, amplification fragment length polymorphism analysis, nucleic acid sequencing, and/or nucleic acid amplification.

In an embodiment, the method comprises
i) obtaining a sample of DNA from a safflower plant, or part thereof such as a seed or cell,
ii) mixing the sample with a pair of primers capable of amplifying a polynucleotide and/or T-DNA defined herein, or a portion thereof, and reagents for nucleic acid amplification,
iii) performing an amplification reaction, and
iv) analysing the product from step iii) for an amplification product.

In an embodiment, the DNA is from a collection of seeds which may or may not comprise a seed of the invention.

In an embodiment, the amplification product spans an integration site of the polynucleotide and/or T-DNA. In a preferred embodiment, the amplification product comprises any one of SEQ ID NOs: 14, 15, 21 or 22.

In an embodiment, the primer pair TTGGATACCGAGGGGAATTTATGGAAC (SEQ ID NO: 35) and CAATCACAATAAGTCGTTGC (SEQ ID NO: 36), or a variant of one or both thereof, for example which is shorter or longer and hybridizes the same region of DNA, is used to detect a polynucleotide which comprises a polynucleotide which comprises a sequence of nucleotides provided as SEQ ID NO:13 and a polynucleotide which comprises a sequence of nucleotides provided as SEQ ID NO:14, such as in line GOR73226, where a 562 bp amplicon is produced.

In an embodiment, the primer pair TTGGATACCGAGGGGAATTTATGGAAC (SEQ ID NO: 37) and TGATAACGATCTTGCGCAAC (SEQ ID NO: 38), or a variant of one or both thereof, for example which is shorter or longer and hybridizes the same region of DNA, is used to detect a polynucleotide which comprises a polynucleotide which comprises a sequence of nucleotides provided as SEQ ID NO:21 and a polynucleotide which comprises a sequence of nucleotides provided as SEQ ID NO:22, such as in line GOR73240 where a 199 bp amplicon is produced.

In an embodiment, the primer pair AAAAGCCTGAACTCACCGC (SEQ ID NO: 39) and TCGTCCATCACAGTTTGCC (SEQ ID NO: 40), or a variant of one or both thereof, for example which is shorter or longer and hybridizes the same region of DNA, is used to detect a gene encoding Hph polypeptide such as in lines GOR73226 and GOR73240 where a 689 bp amplicon is produced.

In an embodiment, the method comprises:
i) obtaining a sample of DNA from a safflower plant, or part thereof such as a seed or cell,
ii) mixing the sample with a probe capable of hybridizing to a polynucleotide and/or T-DNA defined herein, or a portion thereof, and reagents for polynucleotide hybridization,
iii) performing a hybridization reaction, and
iv) analysing the product from step iii) for the probe.

In an embodiment, the DNA is from a collection of seeds which may or may not comprise a seed of the invention.

In an embodiment, the probe hybridizes to a region spanning the integration site of the polynucleotide and/or T-DNA, but will not produce a detectable signal if the polynucleotide and/or T-DNA is not present in the DNA.

In a further aspect, the present invention provides a method of producing safflower seed, the method comprising,
a) growing a plant of the invention, preferably in a field as part of a population of at least 1000 such plants, and
b) harvesting the seed.

In an embodiment, the growing is done in an open field.

In a further aspect, the present invention provides a method of producing safflower oil, comprising obtaining seed of the invention and processing the seed to obtain safflower oil.

In a further aspect, the present invention provides oil obtained from, or obtainable by, one or more of the cell of the invention, the seed of the invention, the safflower plant of the invention, or the method of the invention.

In a further aspect, the present invention provides a composition, preferably a food or feed composition, comprising one or more of the cell of the invention, the seed of the invention, the safflower plant of the invention, the oil of the invention, or the oil produced by a method of the invention, and one or more acceptable carriers.

In a further aspect, the present invention provides for the use of one or more of the cell of the invention, the seed of the invention, the safflower plant of the invention or the oil of the invention, the composition of the invention, or the produced by a method of the invention, for the manufacture of an industrial product.

In a further aspect, the present invention provides a method of producing a feedstuff, the method comprising admixing one or more of the cell of the invention, the seed of the invention, the safflower plant of the invention, the oil of the invention, the composition of the invention, or the oil produced by a method of the invention, with at least one other food ingredient. In a related aspect, the present invention provides a method of producing a feedstuff, the method comprising heating, for example frying, a food product in the presence of the oil of the invention or the composition of the invention, or the oil produced by a method of the invention.

In a further aspect, the present invention provides a feedstuffs, cosmetics or chemicals comprising reacting one or more of the cell of the invention, the seed of the invention, the safflower plant of the invention, the oil of the invention, the composition of the invention, or the oil produced by a method of the invention.

In a further aspect, the present invention provides seedmeal extracted from the safflower seed of the invention. The seedmeal may contain residual oil of the invention, for example if the seed of the invention are crushed to release most of the oil from the seed, or the seedmeal may have been further treated with a solvent to extract residual oil and thereby lack the seedoil, but still comprising DNA which comprises the polynucleotide(s) and/or T-DNAs from the seed.

In another aspect, the present invention provides a process for producing an industrial product, the process comprising the steps of:
i) obtaining one or more of the cell of the invention, the seed of the invention, the safflower plant of the invention, the oil of the invention, the composition of the invention, or the oil produced by a method of the invention,
ii) optionally physically processing one or more of the cell of the invention, the seed of the invention, the safflower plant of the invention, the oil of the invention or the composition of the invention, of step i),
iii) converting at least some of the cell of the invention, the seed of the invention, the safflower plant of the invention, the oil of the invention or the composition of the invention, or the physically processed product of step ii), to the industrial product by applying heat, chemical, or enzymatic means, or any combination thereof, to the lipid, and
iv) recovering the industrial product, thereby producing the industrial product.

In another aspect, the present invention provides a method of producing biofuel, the method comprising
i) reacting one or more of the cell of the invention, the seed of the invention, the safflower plant of the invention, the oil of the invention, the composition of the invention, or the oil produced by a method of the invention, with an alcohol, optionally in the presence of a catalyst, to produce alkyl esters, and
ii) optionally, blending the alkyl esters with petroleum based fuel.

In an embodiment, the alkyl esters are methyl esters.

In a further aspect, the present invention provides a kit comprising primers and/or probes for detecting a polynucleotide and/or T-DNA as defined herein. For instance, the kit may comprise two or more primers defined in Table 1. The kit may also include instructions for use and/or reagents for performing, for example, a DNA amplification reaction and/or for probe hybridization and detection.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e, one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1: Schematic outline of the T-DNA region of transformation vector pCW732. Features include: RB=Right Border: LB=Left Border: Flax linin=flax linin promoter (2033 bp): CtFATB=Carthamus tinctorius L palmitoyl-ACP thioesterase sequence (412 bp): CtFAD2.2=Carthamus tinctorius L. A12 desaturase sequence (756 bp): intl=PDK intron sequence (768 bp): int2=Catalase 1 intron sequence (196 bp): ocs3′=octopine synthase transcription terminator/polyA region (708 bp); nos3′=nopaline synthase transcription terminator/polyA (277 bp): p35S=35S promoter (343 bp): hph=hygromycin phosphotransferase gene including intron (1216 bp).

FIG. 2: Schematic of PCR based genome walking analysis of GOR73226 and GOR73240.

FIG. 3: Schematic genetic map of the T-DNA from pCW732 when inserted into the safflower genome, showing the positions of the PacI and KpnI restriction sites and fragments in the Southern blot hybridisation assay. The location of the rightward PacI and KpnI sites in the flanking genomic DNA was dependant on the site of T-DNA insertion and was unique for each independent transgenic line. The position of the probe is shown. Not drawn to scale.

FIG. 4: Southern blot hybridisation analysis of safflower transformed with the T-DNA from the vector pCW732. DNAs from independent transgenic safflower lines were digested with either KpnI or PacI and probed with a radio-labelled DNA fragment from the protein coding region of the hygromycin phosphotransferase gene. The lanes in order from left to right had DNA digested with KpnI enzyme, S-317 as a non-transgenic negative control: pCW732-40 (GOR73240); pCW732-48 (Event 48); pCW732-22 (Event 22): pCW732-21 (Event 21): pCW732-21 (Event 21, repeated); pCW732-26 (GOR73226): pCW732-30 (Event 30): pCW732-34 (Event 34): pCW732-33 (Event 33). The same order of DNA samples was repeated with PacI digestion, in the lanes to the right. Fragment sizes were estimated from in silico digests.

FIG. 5: Sequence characterisation of the T-DNA insertion of GOR73226. Sequenced amplicons identified from PCR-based genome walking analysis (E26_RB Junction and E26_LB Junction) were aligned to the sequences of the pCW732 vector (pCW732_LB and pCW732_RB) and a draft safflower genomic sequence database (Bower16_13860-7_8).

FIG. 6: Sequence characterisation of the T-DNA insertion of GOR73240. Sequenced amplicons identified from PCR-based genome walking analysis (E40_RB Junction and E40_LB Junction) were aligned to the sequences of the pCW732 vector (pCW732_LB and pCW732_RB) and a draft safflower genomic sequence database (gx_s317 Scaff097804).

FIG. 7: Schematic structure of the T-DNA from pCW732 into safflower to produce transgenic event GOR73226. The schematic map shows 1000 bp upstream and downstream of the insertion site, position of a deletion and the position of the elements of the T-DNA sequences that remain after insertion. E26TF and E26GR are the priming sites for a GOR73226-specific PCR.

FIG. 8: Schematic structure of the GOR73240 insertion of T-DNA into the safflower line CBI1582. The schematic map shows 1000 bp upstream and 960 downstream of the insertion site, duplications, position of a deletion and duplication and the position of the elements of the T-DNA sequences that remain after insertion. E40GF and E40GR are the priming sites of the genome walking analysis. Note that a deletion of 34 bases was also found at the site of the 35 bp duplication. E40_TF and E40_GR2 are priming sites for GOR73240-specific PCR.

FIG. 9: Negative correlation between linoleic and oleic acid in a cross between GOR40 and a linoleic breeding line, see Example 4. The percentage of linoleic acid is negatively correlated with oleic acid.

FIG. 10: Down regulation of CtFAD2.2 and CtFATB in GOR73226 and GOR73240 plants. An average of three biological replicates and three technical replicates were assessed for each event and the non-transgenic safflower control plants. GOR73226 and GOR73240 plants had significantly reduced (p<0.05) levels of transcript abundance for CtFAD2.2 and CtFATB-3 genes compared to the non-transgenic safflower plants. Mean relative mRNA expression levels with the same letter are not significantly different (p>0.05).

FIG. 11: Vigour score for GOR73226 and GOR73240 safflower compared to the parental line. For each site, plots were given a vigour score indicating emergence and establishment where a score of 0 represents no vigour and a score of 9 represents the most vigorous plot. Bars represent the mean vigour score±standard error.

FIG. 12: Plant height of GOR73226 and GOR73240 safflower compared to the parental line. For each site, the height of 10 plants were measured in each plot and the average taken. Bars represent the mean height at each site #standard error.

FIG. 13: Disease incidence scores for GOR73226 and GOR73240 safflower and the parental line. For each site, the incidence of disease on 10 leaves in each plot were assessed where a score of 0 represent a high incidence of disease and a score of 9 represents the most healthy leaves. Bars represent the mean disease score at each site±standard error.

FIG. 14: Insect damage scores for GOR73226 and GOR73240 safflower and the parental line. For each site, the incidence of insect damage on 10 leaves in each plot were assessed where a score of 0 represent a high incidence of insect damage and a score of 9 represents the least damaged leaves. Bars represent the mean disease score at each site±standard error.

FIG. 15: Yield of GOR73226 and GOR73240 safflower compared to the parental line. For each site, the yield of each plot were assessed and converted to T/ha. Bars represent the mean yield at each site±standard error.

### KEY TO THE SEQUENCE LISTING

SEQ ID NO:1-Amino acid sequence of the safflower FAD2-2 polypeptide (CtFAD2-2, Accession No. KC257448.

SEQ ID NO:2—Nucleotide sequence of the cDNA corresponding to the mRNA encoding safflower FAD2-2 polypeptide (CtFAD2-2), without the polyA tail. The translation start codon is at nucleotides 81-83, the translation stop codon at nucleotides 1230-1232, the protein coding region corresponds to nucleotides 81-1229.

SEQ ID NO:3-Amino acid sequence of the safflower FATB-3 polypeptide (CtFATB-3, Accession No. KU059745).

SEQ ID NO:4—Nucleotide sequence of the cDNA corresponding to the mRNA from the safflower FATB-3 gene (CtFATB-3), without the polyA tail. The translation start codon is at nucleotides 237-239, the translation stop codon at nucleotides 1320-1322, the protein coding region corresponds to nucleotides 237-1319; nucleotides 373-784 were used in FATB-3 hpRNA.

SEQ ID NO:5—Nucleotide sequence of the region of the cDNA for the safflower FAD2-2 gene used in the genetic construct pCW732: corresponding to nucleotides 426-1181 of SEQ ID NO:2.

SEQ ID NO:6—Nucleotide sequence of the region of the cDNA for the safflower FATB-3 gene used in the genetic construct pCW732: corresponding to nucleotides 485-784 of SEQ ID NO:4.

SEQ ID NO:7—Nucleotide sequence of the region of the genetic construct pCW732 spanning the T-DNA. Nucleotides 6-168, Right border region, the T-DNA Right border starts at nucleotide 128: nucleotides 296-2328, conlinin promoter: nucleotides 2396-2807, region of CtFATB-3 in antisense orientation: nucleotides 2816-3573, region of CtFAD2-2 in sense orientation: nucleotides 3643-4410, intron from Flaveria trinerva PDK gene, in sense orientation: nucleotides 4445-4634, intron from Cat-1 gene in reverse orientation: nucleotides 4697-5454, region of the CtFAD2-2 gene, in antisense orientation: nucleotides 5463-5874, region of the CtFATB-3 gene in sense orientation; nucleotides 5918-6625, ocs3′ transcription terminator/polyA region: nucleotides 6673-7124, CaMV 35S promoter: nucleotides 7125-8340, Hph coding region including a Cat-1 intron at nucleotides 7464-7653: nucleotides 8366-8640, nos3′ transcription terminator/polyadenylation region: nucleotides 8817-9266, T-DNA left border region, the Left Border ends at nucleotide 9222.

SEQ ID NO:8—Amino acid sequence of the hygromycin phosphotransferase polypeptide (Hph).

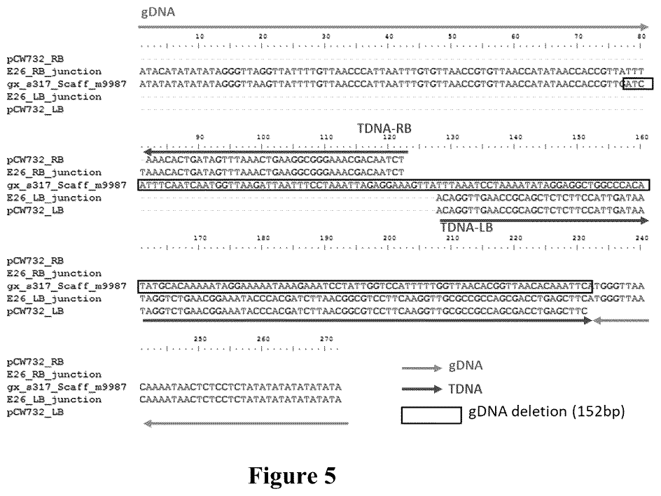

SEQ ID NO:9—Nucleotide sequence of the Right border sequence inserted into GOR73226 (pCW732_RB) (see FIG. 5).

SEQ ID NO:10—Nucleotide sequence of the GOR73226 Right border junction sequence (E26_RB_junction): nucleotides 1-81 correspond to flanking genomic DNA from safflower, nucleotides 82-122 correspond to the Right border sequence of the T-DNA (see FIG. 5).

SEQ ID NO:11—Nucleotide sequence of a Safflower scaffold sequence into which the T-DNA of GOR73226 has inserted (gx_S317_Scaff_m9987): nucleotides 77-231 were deleted during the transformation process to generate GOR73226 (see FIG. 5).

SEQ ID NO: 12—Nucleotide sequence of the GOR73226 Left border junction sequence (E26_LB_junction): nucleotides 1-104 correspond to the Left border sequence of the T-DNA, nucleotides 105-145 correspond to flanking genomic DNA from safflower (see FIG. 5).

SEQ ID NO:13—Nucleotide sequence of the Left border sequence inserted into GOR73226 (E26_LB) (see FIG. 5).

SEQ ID NO:14—Nucleotide sequence of a portion of the GOR73226 Right border junction sequence: nucleotides 1-20 correspond to flanking safflower genomic DNA, nucleotides 21-40 correspond to inserted T-DNA sequence at the Right border.

SEQ ID NO:15—Nucleotide sequence of a portion of the GOR73226 Left border junction sequence: nucleotides 1-20 correspond to inserted T-DNA sequence at the Left border, nucleotides 21-40 correspond to flanking safflower genomic DNA.

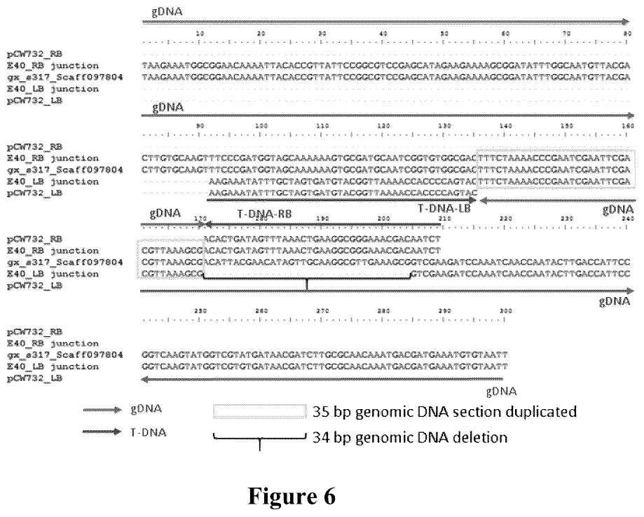

SEQ ID NO:16—Nucleotide sequence of the Right border sequence (pCW732_RB in FIG. 6) inserted into GOR73240.

SEQ ID NO:17—Nucleotide sequence of the GOR73240 Right border junction sequence (E40)_RB_junction): nucleotides 1-170) correspond to flanking genomic DNA.

nucleotides 171-209 correspond to the Right border sequence inserted into GOR73240) (see FIG. 6).

SEQ ID NO:18—Nucleotide sequence of a Safflower scaffold sequence into which the T-DNA of GOR73240) has inserted (gx_S317_Scaff097804): nucleotides 136-170 were duplicated and nucleotides 171-204 were deleted at the Left border junction during the transformation process (see FIG. 6).

SEQ ID NO:19—Nucleotide sequence of the GOR73240 Left border junction sequence (E40)_LB_junction): nucleotides 1-44 correspond to the Left border sequence, nucleotides 45-79 correspond to a 35 bp safflower genomic sequence that was duplicated during the transformation process, nucleotides 80-175 correspond to safflower genomic DNA. A 34 bp genomic sequence was deleted during the transformation process (see FIG. 6).

SEQ ID NO:20—Nucleotide sequence of the Left border of the T-DNA in GOR73240) (pCW732_LB) (see FIG. 6).

SEQ ID NO:21—Nucleotide sequence of a portion of the GOR73240 Right border junction sequence: nucleotides 1-20 correspond to flanking safflower genomic DNA, nucleotides 21-40 correspond to inserted T-DNA sequence at the Right border. SEQ ID NO:22—Nucleotide sequence of a portion of the GOR73240 Left border junction sequence: nucleotides 1-20 correspond to inserted T-DNA sequence at the Left border, nucleotides 21-40 correspond to flanking safflower genomic DNA.

SEQ ID NO's 23 to 32 and 35 to 40—Oligonucleotide primers.

SEQ ID NO:33—Nucleotide sequence of the insertion of the T-DNA in GOR73226 including the flanking safflower genomic sequences. Nucleotides 1-996, safflower genomic sequence upstream of the T-DNA: nucleotides 1002-1042 Right border sequence integrated: nucleotides 1170-3202, conlinin promoter; nucleotides 3270-3681, region of CtFATB-3 in antisense orientation: nucleotides 3690-4445, region of CtFAD2-2 in sense orientation: nucleotides 4517-5284, intron from Flaveria trinerva PDK gene, in sense orientation; nucleotides 5319-5514, intron from Cat-1 gene in reverse orientation: nucleotides 5573-6329, region of the CtFAD2-2 gene, in antisense orientation: nucleotides 6337-6748, region of the CtFATB-3 gene in sense orientation; nucleotides 6792-7499, ocs3′ transcription terminator/poly A region: nucleotides 7645-7987, CaMV 35S promoter: nucleotides 7999-9214, Hph coding region including a Cat-1 intron at nucleotides 8338-8527: nucleotides 9239-9515, nos3′ transcription terminator/polyadenylation region: nucleotides 9691-9851, T-DNA left border region; nucleotides 9852-10056, additional backbone insertion: nucleotides 10057-11060, safflower genomic sequence downstream of the T-DNA.

SEQ ID NO:34—Nucleotide sequence of the insertion of the T-DNA in GOR73240) including the flanking safflower genomic sequences. Nucleotides 1-1059, safflower genomic sequence upstream of the T-DNA: nucleotides 1060-1098, Right border sequence integrated: nucleotides 1226-3258, conlinin promoter: nucleotides 3326-3737, region of CtFATB-3 in antisense orientation: nucleotides 3746-4501, region of CtFAD2-2 in sense orientation: nucleotides 4573-5340), intron from Flaveria trinerva PDK gene, in sense orientation: nucleotides 5375-5570, intron from Cat-1 gene in reverse orientation: nucleotides 5629-6385, region of the CtFAD2-2 gene, in antisense orientation: nucleotides 6393-6804, region of the CtFATB-3 gene in sense orientation; nucleotides 6848-7555, ocs3′ transcription terminator/poly A region: nucleotides 7701-8043. CaMV 35S promoter: nucleotides 8055-9270, Hph coding region including a Cat-1 intron at nucleotides 8394-8583; nucleotides 9295-9571, nos3′ transcription terminator/polyadenylation region: nucleotides 9750-9762, T-DNA left border region; nucleotides 9763-10726, safflower genomic sequence downstream of the T-DNA.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, plant breeding, fatty acid chemistry, gene silencing, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning. John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, more preferably +/−4%, more preferably +/−3%, more preferably +/−2%, more preferably +/−1.5%, more preferably +/−1%, even more preferably +/−0.5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “safflower” refers to members of the species Carthamus tinctorius.

A “line” is a group of plants that displays very little overall variation among individuals sharing that designation. “Line” also refers to a homogeneous assemblage of plants carrying substantially the same genetic material that display little or no genetic variation between individuals for at least one trait. “Variety” or “cultivar” may be used interchangeably with “line,” but in general the former two terms refer to a line that is suitable for commercial production. “Genetically derived” as used for example in the phrase “genetically derived from the parent lines” means that the characteristic in question is dictated wholly or in part by an aspect of the genetic makeup of the plant in question.

An “elite line”, as used herein, is a line selected from a group of lines, obtained by transformation with the same transforming DNA or by back-crossing with plants obtained by such transformation, based on the expression and stability of the transgene construct(s), its compatibility with optimal agronomic characteristics of the plant comprising it, and realization of the desired phenotypic trait. Thus, the criteria for elite event selection are at least one, and advantageously all, of the following:
(a) the presence of the transgene does not unduly compromise other desired characteristics of the plant, such as those relating to agronomic performance or commercial value:
(b) the event is characterized by a well-defined molecular configuration that is stably inherited and for which appropriate diagnostic tools for identity control can be developed:
(c) the genes of interest in the transgene cassette show a correct, appropriate and stable spatial and temporal phenotypic expression, both in heterozygous (or hemizygous) and homozygous condition of the event, at a commercially acceptable level in a range of environmental conditions in which the plants carrying the event are likely to be exposed in normal agronomic use. The foreign DNA may also be associated with a position in the plant genome that allows introgression into further desired commercial genetic backgrounds.

As used herein, GOR73226 refers to a line of safflower plants comprising a polynucleotide which comprises a sequence of nucleotides provided as SEQ ID NO:13, a polynucleotide which comprises a sequence of nucleotides provided as SEQ ID NO:14, and a T-DNA molecule, wherein the T-DNA molecule is inserted into a region of a chromosome of the plant, wherein the region has a nucleotide sequence provided as SEQ ID NO:11. The line is available through the Budapest Treaty under Accession No. ______ at ______ submitted on The Applicant (Commonwealth Scientific and Industrial Research Organisation of Clunies Ross St, Acton ACT 2601, Australian) and their commercial partner (Go Resources of 15 Sutherland Street, Brunswick VIC 3056, Australia) warrant the availability of seed of this line under the same terms as a depository under the Budapest Treaty whenever not available through such a depository.

As used herein, GOR73240 refers to a line of safflower plants comprising a polynucleotide which comprises a sequence of nucleotides provided as SEQ ID NO:21, a polynucleotide which comprises a sequence of nucleotides provided as SEQ ID NO:22 and a T-DNA molecule, wherein the T-DNA molecule is inserted into a region of a chromosome of the plant, wherein the region has a nucleotide sequence provided as SEQ ID NO:18. The line is available through the Budapest Treaty under Accession No. ______ at ______ submitted on The Applicant (Commonwealth Scientific and Industrial Research Organisation of Clunies Ross St, Acton ACT 2601, Australian) and their commercial partner (Go Resources of 15 Sutherland Street, Brunswick VIC 3056, Australia) warrant the availability of seed of this line under the same terms as depository under the Budapest Treaty whenever not available through such a depository.

The terms “seed” and “grain” are related terms as used herein, and have overlapping meanings. “Grain” refers to mature grain such as harvested grain or grain which is still on a plant but ready for harvesting, but can also refer to grain after imbibition or germination, according to the context. Mature grain commonly has a moisture content of less than about 10-12%, for example 6-10% by weight. “Seed” includes “developing seed” as well as “grain” which is mature grain, but not grain after imbibition or germination. “Developing seed” as used herein refers to a seed prior to maturity, typically found in the reproductive structures of the plant after fertilisation or anthesis, but can also refer to such seeds prior to maturity which are isolated from a plant. Seed development in planta is typically divided into early-, mid-, and late phases of development.

“Plant part” includes plant cells, plant organs, plant protoplasts, plant cell tissue cultures from which plants can be regenerated, plant calli, plant clumps, and plant cells that are intact in plants or parts of plants such as embryos, pollen, ovules, seeds, pods, leaves, flowers, branches, fruit, stalks, roots, root tips, anthers, cotyledons, hypocotyls, radicles, single cells, gametes, cell cultures, tissue cultures, and the like. A cotyledon is a type of seed leaf: a small leaf contained on a plant embryo. A cotyledon contains the food storage tissues of the seed. The embryo is a small plant contained within a mature seed. “Plant cells” also encompasses nonregenerable plant cells.

“Progeny” means all descendants including offspring and derivatives of a plant or plants and includes the first, second, third, and subsequent generations: and may be produced by self-pollination or crossing with plants with the same or different genotypes, and may be modified by a range of suitable genetic engineering techniques. Cultigen generally relates to plants that have been deliberately altered and selected by human. “TO” refers to the first generation of transformed plant material, “TI” refers to the seed produced on TO plants, T1 seed gives rise to plants that produce T2 seed, etc., to subsequent Tx progeny.

“Backcrossing” is a process in which a breeder repeatedly crosses hybrid progeny back to a parental line, for example, a first generation hybrid F1 with one of the parental genotypes of the F1 hybrid.

The term “corresponding” refers to a cell, or plant or part thereof (such as a seed) that has the same or similar genetic background as a cell, or plant or part thereof (seed) of the invention but that has not been modified as described herein (for example, the cell, or plant or part thereof lacks a polynucleotide and/or T-DNA as defined herein). A corresponding cell or, plant or part thereof (seed) can be used as a control to compare, for example, CtFAD2-2 protein activity and/or CtFATB-3 protein activity relative with a cell, or plant or part thereof (seed) modified as described herein. A person skilled in the art is able to readily determine an appropriate “corresponding” cell, plant or part thereof (seed) for such a comparison.

As used herein, the terms “FAD2-2” and “CtFAD2-2” and variations thereof refer to a safflower FAD2 polypeptide whose amino acid sequence is provided as SEQ ID NO: 1, such as a polypeptide encoded by nucleotides having a sequence provided as SEQ ID NO:2. As used herein, a FAD2-2 gene is a gene encoding such a polypeptide, or a mutant allele thereof. These terms also include naturally occurring or artificially induced or produced variants of the sequences provided. In an embodiment. FAD2-2 of the invention comprises an amino acid sequence which is at least 95% identical, more preferably at least 99% identical, to the sequence provided as SEQ ID NO:1. CtFAD2-2 genes include alleles which are mutant, that is, that encode polypeptides with altered desaturase activity such as reduced activity, or do not encode functional polypeptides (null alleles). Such alleles may be naturally occurring or induced by artificial mutagenesis. The activity of a CtFAD2-2 gene of the safflower cells of the invention are preferably reduced through RNAi interference, i.e, by addition of a genetic construct that encodes an inhibitory RNA molecule, without modifying the endogenous CtFAD2-2 gene per se. More preferably, the expression of the CtFAD2-2 gene is reduced preferentially in the cells of the developing seed of the safflower plant through the use of a seed-specific promoter, with minimal if any reduction in (FAD2-2 gene expression in tissues other than the seed.

As used herein, the terms “FATB-3” and “CtFATB-3” and variations thereof refer to a safflower FATB polypeptide whose amino acid sequence is provided as SEQ ID NO:3, such as a polypeptide encoded by nucleotides having a sequence provided as SEQ ID NO:4. As used herein, a FATB-3 gene is a gene encoding such a polypeptide or a mutant allele thereof. These terms also include naturally occurring or artificially induced or produced variants of the sequences provided. In an embodiment. FATB-3 of the invention comprises an amino acid sequence which is at least 95% identical, more preferably at least 99% identical, to the sequence provided as SEQ ID NO:3. CtFATB-3 genes include alleles which are mutant, that is, that encode polypeptides with altered palmitoyl-ACP thioesterase activity such as reduced activity, or do not encode functional polypeptides (null alleles). Such alleles may be naturally occurring or induced by artificial mutagenesis. The activity of a CtFATB-3 gene of the safflower cells of the invention are preferably reduced through RNAi interference, i.e, by addition of a genetic construct that encodes an inhibitory RNA molecule, without modifying the endogenous CtFATB-3 gene per se. More preferably, the expression of the CtFATB-3 gene is reduced preferentially in the cells of the developing seed of the safflower plant through the use of a seed-specific promoter, with minimal if any reduction in CtFATB-3 gene expression in tissues other than the seed.

As described herein, the OL locus corresponds to the CtFAD2-1 gene. The oleic acid content of seedoil in olol (homozygous) genotypes was usually 71-75% for greenhouse-grown plants (Knowles, 1989). Knowles (1968) incorporated the ol allele into a safflower breeding program and released the first high oleic (HO) safflower variety “UC-1” in 1966 in the US, which was followed by the release of improved varieties “Oleic Leed” and the Saffola series including Saffola 317 (S-317), S-517 and S-518. The high oleic (olol) genotypes were relatively stable in the oleic acid level when grown at different temperatures in the field (Bartholomew, 1971). In addition, Knowles (1972) also described a different allele ol, at the same locus, which produced in homozygous condition between 35 and 50% oleic acid. In contrast to olol genotype, the oljoli genotype showed a strong response to temperature (Knowles, 1972). As determined herein, the allele of the ol mutation which confers reduced FAD2-1 activity (and overall FAD2 activity) in safflower seed is a mutant FAD2-1 gene comprising the frameshift mutation (due to deletion of a single nucleotide).

As used herein. “T-DNA” refers to for example, T-DNA of an Agrobacterium tumefaciens Ti plasmid or from an Agrobacterium rhizogenes Ri plasmid, or man made variants thereof which function as T-DNA, and to molecules derived therefrom after the T-DNA has been transferred into a safflower cell and integrated into the nuclear genome. The T-DNA may therefor comprise an entire T-DNA including both right and left border sequences, or be derived therefrom, for example by loss of sequences from one or both ends of the molecule through the integration process, including where the right and/or left T-DNA border sequences are lost. The T-DNAs of the invention have inserted into them, anywhere between the right and left border sequences (if present), the polynucleotide of interest. The sequences encoding factors required in trans for transfer of the T-DNA into a plant cell such as vir genes, may be inserted into the T-DNA, or may be present on the same replicon as the T-DNA, or preferably are in trans on a compatible replicon in the Agrobacterium host. Such “binary vector systems” are well known in the art.

As used herein, the term “the same”—for instance in relation to a safflower plant of the invention having “the same “seedling vigour, plant height, time to flowering, harvest lodging, seed crude protein content, seed crude fat content, seed ash content or seed carbohydrate content as a corresponding plant lacking the polynucleotide(s) and/or the T-DNA(s) grown under the same conditions—refers to no statistically significant difference such as having a p-value higher than 0.05 (>0.05).

As used herein, the term “extracted oil” or “extracted lipid” refers to an oil composition which comprises at least 60% (w/w) oil and which has been extracted from a transgenic organism or part thereof.

As used herein, the term “purified” when used in connection with lipid or oil of the invention typically means that that the extracted lipid or oil has been subjected to one or more processing steps of increase the purity of the lipid/oil component. For example, a purification step may comprise one or more or all of the group consisting of: degumming, deodorising, decolourising, drying and/or fractionating the extracted oil. However, as used herein, the term “purified” does not include a transesterification process or other process which alters the fatty acid composition of the lipid or oil of the invention so as to increase the oleic acid content as a percentage of the total fatty acid content. Expressed in other words, the fatty acid composition of the purified lipid or oil is essentially the same as that of the unpurified lipid or oil. The fatty acid composition of the extracted lipid or oil, such as for example the oleic, linoleic and palmitic acid contents, is essentially the same as the fatty acid composition of the lipid or oil in the plant seed from which it is obtained. In this context, “essentially the same” means +/−1%, or, preferably, +/−0.5%. For example, if the oil in the plant seed has 92% oleic acid, the extracted oil has between 91-93% oleic acid.

As used herein, the term “seedoil” refers to a composition obtained from the 20) seed of a plant which comprises at least 60% (w/w) lipid, or obtainable from the seed if the seedoil is still present in the seed. That is, seedoil of, or obtained using, the invention includes seedoil which is present in the seed or portion thereof such as cotyledons or embryo, unless it is referred to as “extracted seedoil” or similar terms in which case it is oil which has been extracted from the seed. The seedoil is preferably extracted seedoil. Seedoil is typically a liquid at room temperature. Preferably, the total fatty acid (TFA) content in the seedoil is >90% oleic acid (C18:149). The fatty acids are typically in an esterified form such as for example, TAG, DAG, acyl-CoA or phospholipid. Unless otherwise stated, the fatty acids may be free fatty acids and/or in an esterified form, preferably >95% or >98% by weight is in the esterified form. In an embodiment, at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99% of the fatty acids in seedoil of the invention can be found as TAG. In an embodiment, seedoil of the invention is “substantially purified” or “purified” oil that has been separated from one or more other lipids, nucleic acids, polypeptides, or other contaminating molecules with which it is associated in the seed or in a crude extract. It is preferred that the substantially purified seedoil is at least 60% free, more preferably at least 75% free, and more preferably, at least 90% free from other components with which it is associated in the seed or extract. Seedoil of the invention may further comprise non-fatty acid molecules such as, but not limited to, sterols such as one or more or all of cholesterol, chalinasterol/24-methylene cholesterol, campesterol/24-methylcholesterol campestanol/24-methylcholestanol, 45-stigmasterol, ergost-7-en-3β-ol, eburicol, β-sitosterol/24-ethylcholesterol, 45-avenasterol/isofucosterol, Δ7-stigmasterol/stigmast-7-en-3β-ol and A7-avenasterol. Seedoil may be extracted from seed by any method known in the art. This typically involves extraction with nonpolar solvents such as hexane, diethyl ether, petroleum ether, chloroform/methanol or butanol mixtures, generally associated with first crushing or rolling of the seeds. Lipids associated with the starch in the grain may be extracted with water-saturated butanol. The seedoil may be “de-gummed” by methods known in the art to remove polysaccharides and/or phospholipids or treated in other ways to remove contaminants or improve purity, stability, or colour. The TAGs and other esters in the seedoil may be hydrolysed to release free fatty acids such as by acid or alkali treatment or by the action of lipases, or the seedoil hydrogenated, treated chemically, or enzymatically as known in the art. However, once the seedoil is processed so that it no longer comprises the TAG, it is no longer considered seedoil as referred to herein.

The free and esterified sterol (for example, sitosterol, campesterol, stigmasterol, brassicasterol, Δ5-avenasterol, sitostanol, campestanol, and cholesterol) concentrations in the purified and/or extracted lipid or oil may be as described in Phillips et al. (2002) and/or as provided in Example 17 of WO 2013/159149. Sterols in plant oils are present as free alcohols, esters with fatty acids (esterified sterols), glycosides and acylated glycosides of sterols. The recovered or extracted seedoils of the invention preferably have between about 100 and about 1000 mg total sterol/100 g of oil. For use as food or feed, it is preferred that sterols are present primarily as free or esterified forms rather than glycosylated forms. In the seedoils of the present invention, preferably at least 50% of the sterols in the oils are present as esterified sterols. The safflower seedoil of the invention preferably has between about 150 and about 400 mg total sterol/100 g, typically about 300 mg total sterol/100 g of seedoil, with sitosterol the main sterol.

As used herein, the term “fatty acid” refers to a carboxylic acid with a long aliphatic tail of at least 8 carbon atoms in length, either saturated or unsaturated. Typically, fatty acids have a carbon-carbon bonded chain of at least 12 carbons in length. Most naturally occurring fatty acids have an even number of carbon atoms because their biosynthesis involves acetate which has two carbon atoms. The fatty acids may be in a free state (non-esterified) or in an esterified form such as part of a TAG, DAG, MAG, acyl-CoA (thio-ester) bound, or other covalently bound form. When covalently bound in an esterified form, the fatty acid is referred to herein as an “acyl” group. The fatty acid may be esterified as a phospholipid such as a phosphatidylcholine (PC), phosphatidylethanolamine, phosphatidylserine, phosphatidylglycerol, phosphatidylinositol, or diphosphatidylglycerol. Saturated fatty acids do not contain any double bonds or other functional groups along the chain. The term “saturated” refers to hydrogen, in that all carbons (apart from the carboxylic acid [—COOH] group) contain as many hydrogens as possible. In other words, the omega (ω) end contains 3 hydrogens (CH3—) and each carbon within the chain contains 2 hydrogens (—CH2—). Unsaturated fatty acids are of similar form to saturated fatty acids, except that one or more alkene functional groups exist along the chain, with each alkene substituting a singly-bonded “—CH2—CH2-” part of the chain with a doubly-bonded “—CH═CH—” portion (that is, a carbon double bonded to another carbon). The two next carbon atoms in the chain that are bound to either side of the double bond can occur in a cis or trans configuration.

“Triacylglyceride” or “TAG” is glyceride in which the glycerol is esterified with three fatty acids. In the Kennedy pathway of TAG synthesis, DAG is formed as described above, and then a third acyl group is esterified to the glycerol backbone by the activity of DGAT. Alternative pathways for formation of TAG include one catalysed by the enzyme PDAT and the MGAT pathway (PCT/AU2011/000794).

As used herein, the term “by weight” refers to the weight of a substance (for example, oleic acid, palmitic acid or linoleic acid) as a percentage of the weight of the composition comprising the substance or a component in the composition. For example, the weight of a particular fatty acid such as oleic acid may be determined as a percentage of the weight of the total fatty acid content of the lipid or seedoil, or the seed.

As used herein, the term “biofuel” refers to any type of fuel, typically as used to power machinery such as automobiles, trucks or petroleum powered motors, whose energy is derived from biological carbon fixation rather than from fossil fuel. Biofuels include fuels derived from biomass conversion, as well as solid biomass, liquid fuels and biogases. Examples of biofuels include bioalcohols, biodiesel, synthetic diesel, vegetable oil, bioethers, biogas, syngas, solid biofuels, algae-derived fuel, biohydrogen, biomethanol, 2,5-Dimethylfuran (DMF), biodimethyl ether (bioDME), Fischer-Tropsch diesel, biohydrogen diesel, mixed alcohols and wood diesel.

As used herein, the term “industrial product” refers to a hydrocarbon product which is predominantly made of carbon and hydrogen such as fatty acid methyl—and/or ethyl-esters or alkanes such as methane, mixtures of longer chain alkanes which are typically liquids at ambient temperatures, a biofuel, carbon monoxide and/or hydrogen, or a bioalcohol such as ethanol, propanol, or butanol, or biochar. The term “industrial product” is intended to include intermediary products that can be converted to other industrial products, for example, syngas is itself considered to be an industrial product which can be used to synthesize a hydrocarbon product which is also considered to be an industrial product. The term industrial product as used herein includes both pure forms of the above compounds, or more commonly a mixture of various compounds and components, for example the hydrocarbon product may contain a range of carbon chain lengths, as well understood in the art.

### Polynucleotides

The terms “polynucleotide”, and “nucleic acid” are used interchangeably. They refer to a polymeric form of nucleotides of any length, either deoxyribonucleotides or ribonucleotides. A polynucleotide defined herein may be of genomic, cDNA, semisynthetic, or synthetic origin, double-stranded or single-stranded and by virtue of its origin or manipulation: (1) is not associated with all or a portion of a polynucleotide with which it is associated in nature, (2) is linked to a polynucleotide other than that to which it is linked in nature, or (3) does not occur in nature. Preferred polynucleotides of the invention encode double-stranded DNA molecules which are capable of being transcribed in plant cells and silencing RNA molecules.

As used herein, the term “gene” is to be taken in its broadest context and includes the deoxyribonucleotide sequences comprising the transcribed region and, if translated, the protein coding region, of a structural gene and including sequences located adjacent to the coding region on both the 5′ and 3′ ends for a distance of at least about 2 kb on either end and which are involved in expression of the gene. In this regard, the gene includes control signals such as promoters, enhancers, termination and/or polyadenylation signals that are naturally associated with a given gene, or heterologous control signals, in which case, the gene is referred to as a “chimeric gene”. The sequences which are located 5′ of the protein coding region and which are present on the mRNA are referred to as 5′ non-translated sequences. The sequences which are located 3′ or downstream of the protein coding region and which are present on the mRNA are referred to as 3′ non-translated sequences. The term “gene” encompasses both cDNA and genomic forms of a gene. A genomic form or clone of a gene contains the coding region which may be interrupted with non-coding sequences termed “introns”, “intervening regions”, or “intervening sequences.” Introns are segments of a gene which are transcribed into nuclear RNA (nRNA). Introns may contain regulatory elements such as enhancers. Introns are removed or “spliced out” from the nuclear or primary transcript: introns therefore are absent in the mRNA transcript. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

An “allele” refers to one specific form of a genetic sequence (such as a gene) within a cell, an individual plant or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”.

A “transgene” is a gene that has been introduced into the genome by a transformation procedure. The transgene may be in an initial transformed plant produced by regeneration from a transformed plant cell or in progeny plants produced by self-fertilisation or crossing from the initial transformant or in plant parts such as seeds. The term “genetically modified” and variations thereof include introducing a gene into a cell by transformation or transduction, mutating a gene in a cell and genetically altering or modulating the regulation of a gene in a cell, or the progeny of any cell modified as described above.

A “genomic region” as used herein refers to a position within the genome where a transgene, or group of transgenes (also referred to herein as a cluster), have been inserted into a cell, or predecessor thereof, such that they are co-inherited in progeny cells after meiosis.

A polynucleotide (or T-DNA) defined herein is a “recombinant polynucleotide” or “exogenous polynucleotide” which refers to a nucleic acid molecule which has been constructed or modified by artificial recombinant methods. The recombinant polynucleotide may be present in a cell in an altered amount or expressed at an altered rate (e.g., in the case of mRNA) compared to its native state. An exogenous polynucleotide is a polynucleotide that has been introduced into a cell that does not naturally comprise the polynucleotide. Typically an exogenous DNA is used as a template for transcription of mRNA which is then translated into a continuous sequence of amino acid residues coding for a polypeptide of the invention within the transformed cell. In another embodiment, part of the exogenous polynucleotide is endogenous to the cell and its expression is altered by recombinant means, for example, an exogenous control sequence is introduced upstream of an endogenous polynucleotide to enable the transformed cell to express the polypeptide encoded by the polynucleotide. For example, an exogenous polynucleotide may express an antisense RNA to an endogenous polynucleotide.

A recombinant polynucleotide of the invention includes polynucleotides which have not been separated from other components of the cell-based or cell-free expression system in which it is present, and polynucleotides produced in said cell-based or cell-free systems which are subsequently purified away from at least some other components. The polynucleotide can be a contiguous stretch of nucleotides existing in nature, or comprise two or more contiguous stretches of nucleotides from different sources (naturally occurring and/or synthetic) joined to form a single polynucleotide. Typically, such chimeric polynucleotides comprise at least an open reading frame operably linked to a promoter suitable of driving transcription of the open reading frame in a cell of interest.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 50%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

A polynucleotide useful for the present invention may selectively hybridise, under stringent conditions, to a polynucleotide defined herein. As used herein, stringent conditions are those that: (1) employ during hybridisation a denaturing agent such as formamide, for example, 50% (v/v) formamide with 0.1% (w/v) bovine serum albumin, 0.1% Ficoll, 0.1% polyvinylpyrrolidone, 50 mM sodium phosphate buffer at pH 6.5 with 750 mM NaCl, 75 mM sodium citrate at 42° C.: or (2) employ 50% formamide, 5×SSC(ω).75 M NaCl, 0.075 M sodium citrate), 50 mM sodium phosphate (pH 6.8), 0.1% sodium pyrophosphate, 5×Denhardt's solution, sonicated salmon sperm DNA (50) g/ml), 0.1% SDS and 10% dextran sulfate at 42° C., in 0.2×SSC and 0.1% SDS, and/or (3) employ low ionic strength and high temperature for washing, for example, 0.015 M NaCl/0.0015 M sodium citrate/0).1% SDS at 50° C.

### RNA Silencing

RNA interference (RNAi) is particularly useful for specifically inhibiting the production of a particular protein such as CtFAD2-2 protein activity and/or CtFATB-3 protein activity as defined herein.

This technology relies on the presence of dsRNA molecules that contain a sequence that is essentially identical to the mRNA of the gene of interest or part thereof and a sequence that is complementary thereto. Conveniently, the dsRNA can be produced from a single promoter in a recombinant vector or host cell, where the sense and anti-sense sequences are covalently joined by a sequence, preferably an unrelated sequence, which enables the sense and anti-sense sequences in the corresponding transcript to hybridize to form the dsRNA molecule with the joining sequence forming a loop structure, although a sequence with identity to the target RNA or its complement can form the loop structure. Typically, the dsRNA is encoded by a double-stranded DNA construct which has sense and antisense sequences in an inverted repeat structure, arranged as an interrupted palindrome, where the repeated sequences are transcribed to produce the hybridising sequences in the dsRNA molecule, and the interrupt sequence is transcribed to form the loop in the dsRNA molecule. The design and production of suitable dsRNA molecules is well within the capacity of a person skilled in the art, particularly considering Waterhouse et al. (1998), Smith et al. (2000), WO 99/32619, WO 99/53050, WO 99/49029, and WO 01/34815.

In one example, a DNA is introduced that directs the synthesis of an at least partly double stranded RNA product(s) with homology, preferably at least 19 consecutive nucleotides complementary to a region of, a target RNA, to be inactivated.

The DNA therefore comprises both sense and antisense sequences that, when transcribed into RNA, can hybridize to form the double stranded RNA region. In one embodiment of the invention, the sense and antisense sequences are separated by a spacer region that comprises an intron which, when transcribed into RNA, is spliced out. This arrangement has been shown to result in a higher efficiency of gene silencing. The double stranded RNA region may comprise one or two RNA molecules, transcribed from either one DNA region or two. The presence of the double stranded molecule is thought to trigger a response from an endogenous system that destroys both the double stranded RNA and also the homologous RNA transcript from the target gene, efficiently reducing or eliminating the activity of the target gene.

The length of the sense and antisense sequences that hybridize should each be at least 19 contiguous nucleotides, corresponding to part of the target mRNA. The full-length sequence corresponding to the entire gene transcript may be used. The degree of identity of the sense and antisense sequences to the targeted transcript should be at least 85%, at least 90%, or at least 95% to 100%. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters.

### Recombinant Cells

The invention also provides a recombinant safflower cell which comprises one or more polynucleotides or T-DNAs defined herein, or combination thereof. The term “recombinant cell” is used interchangeably with the term “transgenic cell” herein. The recombinant cell may be a cell in culture, a cell in vitro, or in a safflower plant or part thereof such as a seed.

### Transformation of Plants

Transgenic plants can be produced using techniques known in the art, such as those generally described in Slater et al., Plant Biotechnology—The Genetic Manipulation of Plants, Oxford University Press (2003), and Christou and Klee, Handbook of Plant Biotechnology, John Wiley and Sons (2004).

As used herein, the terms “stably transforming”, “stably transformed” and variations thereof refer to the integration of the polynucleotide into the genome of the cell such that they are transferred to progeny cells during cell division without the need for positively selecting for their presence. Stable transformants, or progeny thereof, can be selected and/or identified by any means known in the art such as Southern blots on chromosomal DNA, or in situ hybridization of genomic DNA.

Agrobacterium-mediated transfer is a widely applicable system for introducing genes into plant cells because DNA can be introduced into cells in whole plant tissues, plant organs, or explants in tissue culture, for either transient expression, or for stable integration of the DNA in the plant cell genome. The use of Agrobacterium-mediated plant integrating vectors to introduce DNA into plant cells is well known in the art (see for example, U.S. Pat. Nos. 5,177,010, 5,104,310, 5,004,863, or U.S. Pat. No. 5,159,135). The region of DNA to be transferred is defined by the border sequences, and the intervening DNA (T-DNA) is usually inserted into the plant genome. Further, the integration of the T-DNA is a relatively precise process resulting in few rearrangements. In those plant varieties where Agrobacterium-mediated transformation is efficient, it is the method of choice because of the facile and defined nature of the gene transfer. Preferred Agrobacterium transformation vectors are capable of replication in E, coli as well as Agrobacterium, allowing for convenient manipulations as described (Klee et al., In: Plant DNA Infectious Agents, Hohn and Schell, eds., Springer-Verlag, New York, pp. 179-203 (1985)).

Acceleration methods that may be used include for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Such methods are well known in the art. In another embodiment, plastids can be stably transformed. Methods disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985: Toriyama et al., 1986; Abdullah et al., 1986).

Other methods of cell transformation can also be used and include but are not limited to the introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., In: Methods for Plant Molecular Biology, Academic Press, San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polynucleotide is cultivated using methods well known to one skilled in the art.

To confirm the presence of the transgenes in transgenic cells and plants, a polymerase chain reaction (PCR) amplification or Southern blot analysis can be performed using methods known to those skilled in the art. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

A transgenic plant formed using Agrobacterium or other transformation methods typically contains a single transgenic locus on one chromosome. Such transgenic plants can be referred to as being hemizygous for the added gene(s). More preferred is a transgenic plant that is homozygous for the added gene(s), that is, a transgenic plant that contains two added genes, one gene at the same locus on each chromosome of a chromosome pair. A homozygous transgenic plant can be obtained by self-fertilising a hemizygous transgenic plant, germinating some of the seed produced and analyzing the resulting plants for the gene of interest.

It is also to be understood that two different transgenic plants that contain two independently segregating exogenous genes or loci can also be crossed (mated) to produce offspring that contain both sets of genes or loci. Selfing of appropriate F1 progeny can produce plants that are homozygous for both exogenous genes or loci.

Back-crossing to a parental plant and out-crossing with a non-transgenic plant are also contemplated, as is vegetative propagation. Descriptions of other breeding methods that are commonly used for different traits and crops can be found in Fehr, In: Breeding Methods for Cultivar Development, Wilcox J, ed., American Society of Agronomy, Madison Wis. (1987).

For the transformation of safflower, particularly useful methods are described by Belide et al. (2011).

Marker Assisted Selection Marker assisted selection is a well recognised method of selecting for heterozygous plants required when backcrossing with a recurrent parent in a classical breeding program. The population of plants in each backcross generation will be heterozygous for the gene of interest normally present in a 1:1 ratio in a backcross population, and the molecular marker can be used to distinguish the two alleles of the gene. By extracting DNA from, for example, young shoots and testing with a specific marker for the introgressed desirable trait, early selection of plants for further backcrossing is made whilst energy and resources are concentrated on fewer plants. To further speed up the backcrossing program, the embryo from immature seeds (25 days post anthesis) may be excised and grown up on nutrient media under sterile conditions, rather than allowing full seed maturity. This process, termed “embryo rescue”, used in combination with DNA extraction at the three leaf stage and analysis for the desired genotype allows rapid selection of plants carrying the desired trait, which may be nurtured to maturity in the greenhouse or field for subsequent further backcrossing to the recurrent parent.

Any molecular biological technique known in the art which is capable of detecting a polynucleotide can be used in the methods of the present invention. Such methods include, but are not limited to, the use of nucleic acid amplification, nucleic acid sequencing, nucleic acid hybridization with suitably labeled probes, single-strand conformational analysis (SSCA), denaturing gradient gel electrophoresis (DGGE), heteroduplex analysis (HET), chemical cleavage analysis (CCM), catalytic nucleic acid cleavage or a combination thereof (see, for example, Lemieux, 2000: Langridge et al., 2001).

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme.

Methods for PCR are known in the art, and are taught, for example, in “PCR” (Ed. M. J. McPherson and S. G Moller (2000) BIOS Scientific Publishers Ltd, Oxford). PCR can be performed on cDNA obtained from reverse transcribing mRNA isolated from plant cells. However, it will generally be easier if PCR is performed on genomic DNA isolated from a plant.

A primer is an oligonucleotide sequence that is capable of hybridising in a sequence specific fashion to the target sequence and being extended during the PCR.

Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al. (supra) and Sambrook et al. (supra). Sequencing can be carried out by any suitable method, for example, dideoxy sequencing, chemical sequencing or variations thereof. Direct sequencing has the advantage of determining variation in any base pair of a particular sequence.

Hybridization based detection systems include, but are not limited to, the TaqMan assay and molecular beacon assay (U.S. Pat. No. 5,925,517). The TaqMan assay (U.S. Pat. No. 5,962,233) uses allele specific (ASO) probes with a donor dye on one end and an acceptor dye on the other end such that the dye pair interact via fluorescence resonance energy transfer (FRET).

In one embodiment, the method described in Example 3 is used in selection and breeding programs to identify and select safflower plants with the ol mutation. For instance, the method comprises performing an amplification reaction on genomic DNA obtained from the plant using primers outlined in Table 1.

Production of Lipids and/or Oils High in Oleic Acid

Techniques that are routinely practiced in the art can be used to extract, process, purify and analyze the lipids produced by the plants, in particular the seeds, of the instant invention. Such techniques are described and explained throughout the literature in sources such as, Fereidoon Shahidi, Current Protocols in Food Analytical Chemistry, John Wiley & Sons, Inc. (2001) D1.1.1-D1.1.11, and Perez-Vich et al. (1998).

### Production of Seedoil

Typically, plant seeds are cooked, pressed, and/or extracted to produce crude seedoil, which is then degummed, refined, bleached, and deodorized. Generally, techniques for crushing seed are known in the art. For example, safflower seed can be tempered by spraying them with water to raise the moisture content to, for example, 8.5%, and flaked using a smooth roller with a gap setting of 0.23 to 0.27 mm. Depending on the type of seed, water may not be added prior to crushing. Application of heat deactivates enzymes, facilitates further cell rupturing, coalesces the lipid droplets, and agglomerates protein particles, all of which facilitate the extraction process.

In an embodiment, the majority of the seedoil is released by passage through a screw press. Cakes expelled from the screw press may then be solvent extracted for example, with hexane, using a heat traced column. Alternatively, crude seedoil produced by the pressing operation can be passed through a settling tank with a slotted wire drainage top to remove the solids that are expressed with the seedoil during the pressing operation. The solid residue from the pressing and extraction, after removal of the hexane, is the seedmeal, which is typically used as animal feed. The clarified seedoil can be passed through a plate and frame filter to remove any remaining fine solid particles. If desired, the seedoil recovered from the extraction process can be combined with the clarified seedoil to produce a blended crude seedoil.

Once the solvent is stripped from the crude seedoil, the pressed and extracted portions are combined and subjected to normal lipid processing procedures such as, for example, degumming, caustic refining, bleaching, and deodorization. Some or all steps may be omitted, depending on the nature of the product path, e.g. for feed grade oil, limited treatment may be needed whereas for oleochemical applications, more purification steps are required.

### Degumming

Degumming is an early step in the refining of oils and its primary purpose is the removal of most of the phospholipids from the oil, which may be present as approximately 1-2% of the total extracted lipid. Addition of ˜2% of water, typically containing phosphoric acid, at 70-80° C., to the crude oil results in the separation of most of the phospholipids accompanied by trace metals and pigments. The insoluble material that is removed is mainly a mixture of phospholipids and triacylglycerols and is also known as lecithin. Degumming can be performed by addition of concentrated phosphoric acid to the crude seedoil to convert non-hydratable phosphatides to a hydratable form, and to chelate minor metals that are present. Gum is separated from the seedoil by centrifugation.

### Alkali Refining

Alkali refining is one of the refining processes for treating crude oil, sometimes also referred to as neutralization. It usually follows degumming and precedes bleaching. Following degumming, the seedoil can treated by the addition of a sufficient amount of an alkali solution to titrate all of the fatty acids and phosphoric acids, and removing the soaps thus formed. Suitable alkaline materials include sodium hydroxide, potassium hydroxide, sodium carbonate, lithium hydroxide, calcium hydroxide, calcium carbonate and ammonium hydroxide. This process is typically carried out at room temperature and removes the free fatty acid fraction. Soap is removed by centrifugation or by extraction into a solvent for the soap, and the neutralised oil is washed with water. If required, any excess alkali in the oil may be neutralized with a suitable acid such as hydrochloric acid or sulphuric acid.

### Bleaching

Bleaching is a refining process in which oils are heated at 90-120° C. for 10-30 minutes in the presence of a bleaching earth (0.2-2.0%) and in the absence of oxygen by operating with nitrogen or steam or in a vacuum. This step in oil processing is designed to remove unwanted pigments (carotenoids, chlorophyll, gossypol etc), and the process also removes oxidation products, trace metals, sulphur compounds and traces of soap.

### Deodorization

Deodorization is a treatment of oils and fats at a high temperature (200-260° C.) and low pressure (0.1-1 mm Hg). This is typically achieved by introducing steam into the seedoil at a rate of about 0.1 ml/minute/100 ml of seedoil. After about 30 minutes of sparging, the seedoil is allowed to cool under vacuum. The seedoil is typically transferred to a glass container and flushed with argon before being stored under refrigeration. This treatment improves the colour of the seedoil and removes a majority of the volatile substances or odorous compounds including any remaining free fatty acids, monoacylglycerols and oxidation products.

### Winterisation

Winterization is a process sometimes used in commercial production of oils for the separation of oils and fats into solid (stearin) and liquid (olein) fractions by crystallization at sub-ambient temperatures. It was applied originally to cottonseed oil to produce a solid-free product. It is typically used to decrease the saturated fatty acid content of oils.

### Transesterification

Transesterification is a process that exchanges the fatty acids within and between TAGs, initially by releasing fatty acids from the TAGs either as free fatty acids or as fatty acid esters, usually fatty acid ethyl esters. When combined with a fractionation process, transesterification can be used to modify the fatty acid composition of lipids (Marangoni et al., 1995). Transesterification can use either chemical or enzymatic means, the latter using lipases which may be position-specific (sn-⅓ or sn-2 specific) for the fatty acid on the TAG, or having a preference for some fatty acids over others. The fatty acid fractionation to increase the concentration of LC-PUFA in an oil can be achieved by any of the methods known in the art, such as, for example, freezing crystallization, complex formation using urea, molecular distillation, supercritical fluid extraction and silver ion complexing. Complex formation with urea is a preferred method for its simplicity and efficiency in reducing the level of saturated and monounsaturated fatty acids in the oil (Gamez et al., 2003). Initially, the TAGs of the oil are split into their constituent fatty acids, often in the form of fatty acid esters, by hydrolysis or by lipases and these free fatty acids or fatty acid esters are then mixed with an ethanolic solution of urea for complex formation. The saturated and monounsaturated fatty acids easily complex with urea and crystallize out on cooling and may subsequently be removed by filtration. The non-urea complexed fraction is thereby enriched with LC-PUFA.

### Hydrogenation

Hydrogenation of fatty acids involves treatment with hydrogen, typically in the presence of a catalyst. Non-catalytic hydrogenation takes place only at very high temperatures.

Hydrogenation is commonly used in the processing of plant oils. Hydrogenation converts unsaturated fatty acids to saturated fatty acids, and in some cases, trans fats. Hydrogenation results in the conversion of liquid plant oils to solid or semi-solid fats, such as those present in margarine. Changing the degree of saturation of the fat changes some important physical properties such as the melting range, which is why liquid oils become semi-solid. Solid or semi-solid fats are preferred for baking because the way the fat mixes with flour produces a more desirable texture in the baked product. Because partially hydrogenated vegetable oils are cheaper than animal source fats, are available in a wide range of consistencies, and have other desirable characteristics (e.g., increased oxidative stability/longer shelf life), they are the predominant fats used as shortening in most commercial baked goods.

In an embodiment, the lipid/oil of the invention has not been hydrogenated. An indication that a lipid or oil has not been hydrogenated is the absence of any trans fatty acids in its TAG.

### Uses of Oils

The lipids/oils such as the seedoil, preferably the safflower seedoil, produced by the methods described herein have a variety of uses. In some embodiments, the lipids are used as food oils. In other embodiments, the lipids are refined and used as lubricants or for other industrial uses such as the synthesis of plastics. It may be used in the manufacture of cosmetics, soaps, fabric softeners, electrical insulation or detergents. It may be used to produce agricultural chemicals such as surfactants or emulsifiers. In some embodiments, the lipids are refined to produce biodiesel. The oil of the invention may advantageously be used in paints or varnishes since the absence of linolenic acid means it does not discolour easily.

An industrial product produced using a method of the invention may be a hydrocarbon product such as fatty acid esters, preferably fatty acid methyl esters and/or a fatty acid ethyl esters, an alkane such as methane, ethane or a longer-chain alkane, a mixture of longer chain alkanes, an alkene, a biofuel, carbon monoxide and/or hydrogen gas, a bioalcohol such as ethanol, propanol, or butanol, biochar, or a combination of carbon monoxide, hydrogen and biochar. The industrial product may be a mixture of any of these components, such as a mixture of alkanes, or alkanes and alkenes, preferably a mixture which is predominantly (>50%) C4-C8 alkanes, or predominantly C6 to C10 alkanes, or predominantly C6 to C8 alkanes. The industrial product is not carbon dioxide and not water, although these molecules may be produced in combination with the industrial product. The industrial product may be a gas at atmospheric pressure/room temperature, or preferably, a liquid, or a solid such as biochar, or the process may produce a combination of a gas component, a liquid component and a solid component such as carbon monoxide, hydrogen gas, alkanes and biochar, which may subsequently be separated. In an embodiment, the hydrocarbon product is predominantly fatty acid methyl esters. In an alternative embodiment, the hydrocarbon product is a product other than fatty acid methyl esters.

Heat may be applied in the process, such as by pyrolysis, combustion, gasification, or together with enzymatic digestion (including anaerobic digestion, composting, fermentation). Lower temperature gasification takes place at, for example, between about 700° C., to about 1000° C. Higher temperature gasification takes place at, for example, between about 1200° C., to about 1600° C. Lower temperature pyrolysis (slower pyrolysis), takes place at about 400° C., whereas higher temperature pyrolysis takes place at about 500° C. Mesophilic digestion takes place between about 20° C., and about 40° C. Thermophilic digestion takes place from about 50° C., to about 65° C.

Chemical means include, but are not limited to, catalytic cracking, anaerobic digestion, fermentation, composting and transesterification. In an embodiment, a chemical means uses a catalyst or mixture of catalysts, which may be applied together with heat. The process may use a homogeneous catalyst, a heterogeneous catalyst and/or an enzymatic catalyst. In an embodiment, the catalyst is a transition metal catalyst, a molecular sieve type catalyst, an activated alumina catalyst or sodium carbonate as a catalyst. Catalysts include acid catalysts such as sulphuric acid, or alkali catalysts such as potassium or sodium hydroxide or other hydroxides. The chemical means may comprise transesterification of fatty acids in the lipid, which process may use a homogeneous catalyst, a heterogeneous catalyst and/or an enzymatic catalyst. The conversion may comprise pyrolysis, which applies heat and may apply chemical means, and may use a transition metal catalyst, a molecular sieve type catalyst, an activated alumina catalyst and/or sodium carbonate as a catalyst.

Enzymatic means include, but are not limited to, digestion by microorganisms in, for example, anaerobic digestion, fermentation or composting, or by recombinant enzymatic proteins.

### Feedstuffs

The lipid/oil of the invention has advantages in food applications because of its very high oleic acid content and the low levels of linoleic acid (<3.2%) and saturated fatty acids such as palmitic acid, and the essentially zero level of linolenic acid. This provides the oil with a high oxidative stability, producing less rancidity and making it ideal for food applications where heating is required, such as in frying applications, for example for French fries. The oil has a high OSI (oxidative stability index) which is measured as the length of time an oil may be held at 110° C., such as greater than 20 or hours, preferably greater than 30 hours or greater than 50 hours. The low levels of saturated fatty acids relative to other vegetable oils provides for health benefits since saturated fatty acids have been associated with deleterious effects on health. The oils also have essentially zero trans fatty acid content which is desirable in some markets as trans fatty acids have also been associated with negative effects on heart health or raising LDL cholesterol. Moreover, due to its very low level of polyunsaturated fatty acids, the oil does not require hydrogenation to lower the levels of PUFA—such hydrogenation produces trans fatty acids. The oils are also advantageous for reducing the incidence or severity of obesity and diabetes. They are also desirable for food applications in that they contain only naturally occurring fatty acids (Scarth and Tang, 2006).

For purposes of the present invention, “feedstuffs” include any food or preparation for human or animal consumption (including for enteral and/or parenteral consumption) which when taken into the body: (1) serve to nourish or build up tissues or supply energy, and/or (2) maintain, restore or support adequate nutritional status or metabolic function. Feedstuffs of the invention include nutritional compositions for babies and/or young children.

Feedstuffs of the invention comprise for example, a cell of the invention, a plant of the invention, the plant part of the invention, the seed of the invention, an extract of the invention, the product of a method of the invention, or a composition along with a suitable carrier(s). The term “carrier” is used in its broadest sense to encompass any component which may or may not have nutritional value. As the person skilled in the art will appreciate, the carrier must be suitable for use (or used in a sufficiently low concentration) in a feedstuff, such that it does not have deleterious effect on an organism which consumes the feedstuff.

The feedstuff of the present invention comprises a lipid produced directly or indirectly by use of the methods, cells or organisms disclosed herein. The composition may either be in a solid or liquid form. Additionally, the composition may include edible macronutrients, vitamins, and/or minerals in amounts desired for a particular use. The amounts of these or other ingredients will vary depending on whether the composition is intended for use with normal individuals or for use with individuals having specialized needs such as individuals suffering from metabolic disorders and the like.

The foods may be produced by mixing the oil with one or more other ingredients so that the food comprises the oil, or mixed with one or more other ingredients to make a food additive such as salad dressing or mayonnaise. The food or food additive may comprise 1%-10% or more of the oil by weight. The oil may be blended with other vegetable oils to provide for optimal composition or with solid fats or with palm oil to provide semisolid shortening. Foods or food additives produced from the oil include salad dressing, mayonnaise, margarine, bread, cakes, biscuits (cookies), croissants, baked goods, pancakes or pancake mixes, custards, frozen desserts, non-dairy foods.

Examples of suitable carriers with nutritional value include, but are not limited to, macronutrients such as edible fats, carbohydrates and proteins. Examples of such edible fats include, but are not limited to, coconut oil, borage oil, fungal oil, black current oil, soy oil, and mono- and di-glycerides. Examples of such carbohydrates include, but are not limited to, glucose, edible lactose, and hydrolyzed starch. Additionally, examples of proteins which may be utilized in the nutritional composition of the invention include, but are not limited to, soy proteins, electrodialysed whey, electrodialysed skim milk, milk whey, or the hydrolysates of these proteins. With respect to vitamins and minerals, the following may be added to the feedstuff compositions of the present invention, calcium, phosphorus, potassium, sodium, chloride, magnesium, manganese, iron, copper, zinc, selenium, iodine, and vitamins A, E, D, C, and the B complex. Other such vitamins and minerals may also be added.

The components utilized in the feedstuff compositions of the present invention can be of semi-purified or purified origin. By semi-purified or purified is meant a material which has been prepared by purification of a natural material.

A feedstuff composition of the present invention may also be added to food even when supplementation of the diet is not required. For example, the composition may be added to food of any type, including, but not limited to, margarine, modified butter, cheeses, milk, yogurt, chocolate, candy, snacks, salad oils, cooking oils, cooking fats, meats, fish and beverages.

Additionally, lipid produced in accordance with the present invention or host cells transformed to contain and express the subject genes may also be used as animal food supplements to alter an animal's tissue or milk fatty acid composition or fatty acid composition of eggs, to one more desirable for human or animal consumption, or for animal health and wellbeing. Examples of such animals include sheep, cattle, horses, poultry, pets such as dogs and cats and the like.

Furthermore, feedstuffs of the invention can be used in aquaculture to increase the levels of fatty acids in fish for human or animal consumption.

Preferred feedstuffs of the invention are the plants, seed and other plant parts such as leaves, fruits and stems which may be used directly as food or feed for humans or other animals. For example, animals may graze directly on such plants grown in the field, or be fed more measured amounts in controlled feeding.

### Compositions

The present invention also encompasses compositions, particularly pharmaceutical compositions, comprising one or more lipids or oils produced using the methods of the invention.

A pharmaceutical composition may comprise one or more of the lipids, in combination with a standard, well-known, non-toxic pharmaceutically-acceptable carrier, adjuvant or vehicle such as phosphate-buffered saline, water, ethanol, polyols, vegetable oils, a wetting agent, or an emulsion such as a water/oil emulsion. The composition may be in either a liquid or solid form. For example, the composition may be in the form of a tablet, capsule, ingestible liquid, powder, topical ointment or cream. Proper fluidity can be maintained for example, by the maintenance of the required particle size in the case of dispersions and by the use of surfactants. It may also be desirable to include isotonic agents for example, sugars, sodium chloride, and the like. Besides such inert diluents, the composition can also include adjuvants such as wetting agents, emulsifying and suspending agents, sweetening agents, flavoring agents and perfuming agents.

Suspensions, in addition to the active compounds, may comprise suspending agents such as ethoxylated isostearyl alcohols, polyoxyethylene sorbitol and sorbitan esters, microcrystalline cellulose, aluminum metahydroxide, bentonite, agar-agar, and tragacanth, or mixtures of these substances.

Solid dosage forms such as tablets and capsules can be prepared using techniques well known in the art. For example, lipid produced in accordance with the present invention can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and the relevant lipid(s).

For intravenous administration, the lipids produced in accordance with the present invention or derivatives thereof may be incorporated into commercial formulations.

A typical dosage of a particular fatty acid is from 0.1 mg to 20 g, taken from one to five times per day (up to 100 g daily) and is preferably in the range of from about 10 mg to about 1, 2, 5, or 10 g daily (taken in one or multiple doses). As known in the art, a minimum of about 300 mg/day of fatty acid is desirable. However, it will be appreciated that any amount of fatty acid will be beneficial to the subject.

Possible routes of administration of the pharmaceutical compositions of the present invention include for example, enteral and parenteral. For example, a liquid preparation may be administered orally. Additionally, a homogenous mixture can be completely dispersed in water, admixed under sterile conditions with physiologically acceptable diluents, preservatives, buffers or propellants to form a spray or inhalant.

The dosage of the composition to be administered to the subject may be determined by one of ordinary skill in the art and depends upon various factors such as weight, age, overall health, past history, immune status, etc., of the subject.

Additionally, the compositions of the present invention may be utilized for cosmetic purposes. The compositions may be added to pre-existing cosmetic compositions, such that a mixture is formed, or a fatty acid produced according to the invention may be used as the sole “active” ingredient in a cosmetic composition.

### EXAMPLES

### Example 1. General Materials and Methods

### Plant Materials and Growth Conditions

A non-transgenic safflower (Carthamus tinctorius) line designated herein as CBI1582 was obtained by selection from a heterogeneous population of some breeding material received from Mexico. Approximately 20 seed from each breeding population were sown and the resultant plants grown in quarantine facilities for one season. Seeds from the plants were retained and evaluated non-destructively for fatty acid profile and oil content. A high oleic acid line was selected that produced at least 80% oleic acid in the seedoil. Since progeny plants of that line still appeared to be heterogeneous in morphology, further selections were made through two more generations by single seed descent. A line from the fourth generation that was stable in phenotype was retained as CBI1582 and used in subsequent genetic transformation experiments. The CBI1582 line is available from CSIRO, Canberra, Australia.

Safflower plants genotypes such as CBI1582, S-317 and transformants of CBI1582 were grown from seed in a glasshouse in a perlite and sandy loam potting mix under a day/night cycle of 16 hrs (25° C.)/8 hrs)(22° C.. Plants were also grown in the field as described below, at various locations in Victoria, New South Wales, Queensland and Western Australia, Australia.

Plant tissues for DNA and RNA extraction including leaves, roots, cotyledons and hypocotyls were harvested from safflower seedlings 10 days post-germination unless otherwise stated. Flowering heads were obtained at the first day of flower opening and developing embryos were harvested at three developmental stages at 7 (early), 15 (middle) and 20 (late) days post anthesis (DPA). Samples were immediately chilled in liquid nitrogen and stored at −80° C. until DNA and RNA extraction was carried out.

### Lipid Analysis

Isolation of Lipid Samples from Single Seeds for Rapid Fatty Acid Composition Analysis

After being harvested at plant maturity, safflower seeds were dried by storing the seeds for 3 days at 37° C., and subsequently at room temperature if not analysed right away. Single seeds or pooled seeds were crushed between small filter papers and the exuded seedoil samples that soaked into the papers were analysed for fatty acid composition by GC methods as described below.

Total Lipid Isolation from Half Cotyledons Post Germination

For screening purposes, for example for progeny seeds from transgenic plants, safflower seeds were germinated on a wet filter paper in a petri dish for 1 day. A cotyledon was carefully removed from each germinated seed for lipid analysis as described above for mature whole seeds. The remainder of each seedling was transferred to soil and the resultant plants grown to maturity followed by harvesting of seeds to maintain the transgenic line.

Extraction of Oil from Seeds Using Soxhlet Apparatus

For quantitative extraction of seedoil, harvested safflower seeds were dried in an oven at 105° C., overnight and then ground in a Puck Mill for 1 min. The ground seed material (˜250 grams) was collected into a pre-weighed thimble and weighed prior to oil extraction. After adding a layer of cotton wool on top of the meal, the oil was extracted in a Soxhlet Extraction apparatus with solvent (Petroleum Spirit 40-60 C), initially at 70-80° C. The mixture was then refluxed overnight with the solvent syphoning to the extraction flask every 15-20 min. The dissolved, extracted oil was recovered by evaporating off the solvent using a rotary evaporator under vacuum. The weight of the extracted oil was measured and the oil content was determined. To determine the fatty acid composition of the extracted oil, small aliquots were diluted in chloroform and analysed by gas chromatography.

### Fractionation of Lipids

When required, TAG fractions were separated from other lipid components using a 2-phase thin-layer chromatography (TLC) system on pre-coated silica gel plates (Silica gel 60, Merck). An extracted lipid sample equivalent to 10 mg dry weight of plant tissue was chromatographed in a first phase with hexane/diethyl ether (98/2 v/v) to remove non-polar waxes and then in a second phase using hexane/diethyl ether/acetic acid (70/30/1 v/v/v).

When required, polar lipids were separated from non-polar lipids in lipid samples extracted from an equivalent of 5 mg dry weight of leaves using two-dimensional TLC (Silica gel 60, Merck), using chloroform/methanol/water (65/25/4 v/v/v) for the first direction and chloroform/methanol/NH₄OH/ethylpropylamine (130/70/10/1 v/v/v/v) for the second direction.

The lipid spots, and appropriate standards run on the same TLC plates, were visualized by brief exposure to iodine vapour, collected into vials and transmethylated to produce FAME for GC analysis as follows.

### Fatty Acid Methyl Esters (FAME) Preparation and Gas Chromatography (GC) Analysis

For fatty acid composition analysis by GC, extracted lipid samples prepared as described above were transferred to a glass tube and transmethylated in 2 mL of 1 M HCl in methanol (Supelco) at 80° C. for 3 hours. After cooling to room temperature, 1.3 mL 0.9% NaCl and 800 μL hexane were added to each tube and FAMEs were extracted into the hexane phase. To determine the fatty acid composition, the FAMEs were separated by gas-chromatography (GC) using an Agilent Technologies 7890A gas chromatograph (Palo Alto, California, USA) equipped with a 30-m BPX70 column essentially as described by Zhou et al. (2011) except that the temperature ramping program was changed to initial temperature at 150° C., holding for 1 min, ramping 3° C./min to 210° C., then 50° C./min to 240° C. for a final holding of 2 min. Peaks were quantified with Agilent Technologies ChemStation software (Rev B.03.01 (317), Palo Alto, California, USA). Peak responses were similar for the fatty acids of authentic Nu-Chek GLC standard-411 (Nu-Chek Prep Inc, MN, USA) which contained equal proportions of 31 different fatty acid methyl esters, including 18:1, 18:0, 20:0 and 22:0 was used for calibration. The proportion of each fatty acid in the samples was calculated on the basis of individual and total peaks areas for the fatty acids.

### Analysis of the Sterol Content of Oil Samples

Samples of approximately 10 mg of oil together with an added aliquot of C24:0 monol as an internal standard were saponified using 4 mL 5% KOH in 80% MeOH and heating for 2 h at 80° C., in a Teflon-lined screw-capped glass tube. After the reaction mixture was cooled, 2 mL of Milli-Q water was added and the sterols extracted into 2 mL of hexane: dichloromethane (4:1 v/v) by shaking and vortexing. The mixture was centrifuged and the sterol extract was removed and washed with 2 mL of Milli-Q water. The sterol extract was then removed after shaking and centrifugation. The extract was evaporated using a stream of nitrogen gas and the sterols silylated using 200 mL of BSTFA and heating for 2 h at 80° C.

For GC/GC-MS analysis of the sterols, sterol-OTMSi derivatives were dried under a stream of nitrogen gas on a heat block at 40° C. and then re-dissolved in chloroform or hexane immediately prior to GC/GC-MS analysis. The sterol-OTMS derivatives were analysed by gas chromatography (GC) using an Agilent Technologies 6890A GC (Palo Alto, California, USA) fitted with an Supelco Equity™-1 fused silica capillary column (15 m×0.1 mm i.d., 0.1 μm film thickness), an FID, a split/splitless injector and an Agilent Technologies 7683B Series auto sampler and injector. Helium was the carrier gas. Samples were injected in splitless mode at an oven temperature of 120° C. After injection, the oven temperature was raised to 270° C., at 10° C. min 1 and finally to 300° C., at 5° C. min 1. Peaks were quantified with Agilent Technologies ChemStation software (Palo Alto, California, USA). GC results are subject to an error of +5% of individual component areas.

GC-mass spectrometric (GC-MS) analyses were performed on a Finnigan Thermoquest GCQ GC-MS and a Finnigan Thermo Electron Corporation GC-MS: both systems were fitted with an on-column injector and Thermoquest Xcalibur software (Austin, Texas, USA). Each GC was fitted with a capillary column of similar polarity to that described above. Individual components were identified using mass spectral data and by comparing retention time data with those obtained for authentic and laboratory standards. A full procedural blank analysis was performed concurrent to the sample batch.

### Example 2. Generation and Selection of GOR73226 and GOR73240 Safflower

Safflower plants of the CBI1582 genotype were transformed with a genetic construct to produce seeds and seedoil having between 90% and 95% oleic acid in the total fatty acid content. To do this, two genes were selected for down-regulation by RNA interference (RNAi), namely the FAD2-2 and FATB-3 genes of safflower.

The FAD2-2 gene was one of eleven FAD2-like genes found in safflower (Cao et al., 2013), encoding a fatty acid 412 desaturase enzyme that converts the monounsaturated oleic acid into linoleic acid, a polyunsaturated fatty acid. The amino acid sequence of the FAD2-2 polypeptide is provided herein as SEQ ID NO: 1 and the nucleotide sequence of the cDNA corresponding to the mRNA transcript from the FAD2-2 gene is provided as SEQ ID NO:2.

The FATB-3 gene is one of three FATB-like genes in safflower, encoding a acyl-ACP thioesterase enzyme that hydrolyses a thioester bond in palmityl-ACP to release palmitic acid during fatty acid synthesis in the plastids. The amino acid sequence of the FATB-3 polypeptide is provided herein as SEQ ID NO:3 and the nucleotide sequence of the cDNA corresponding to the mRNA transcript from the FATB-3 gene is provided as SEQ ID NO:4.

Regions of 756 nucleotides of the FAD2-2 cDNA and 412 nucleotides of the FATB-3 cDNA were selected to make the RNAi construct. The nucleotide sequences of these target regions are provided herein as SEQ ID NO:5 and SEQ ID NO:6, respectively. DNA fragments having these sequences were used to generate an inverted repeat construct that, when transcribed in the safflower cells, produced a hairpin RNA having a double-stranded RNA (dsRNA) region corresponding to the selected target gene sequences. The inverted repeat region was placed under the control of a seed-specific promoter from a flax conlinin gene (U.S. Pat. No. 7,642,346) and a transcription terminator/polyadenylation region from an octopine synthase gene (ocs3′). The target sequences (sense and antisense) in the inverted repeat were separated by two intron sequences, one an intron from a Flaveria trinerva PDK gene in the sense orientation with respect to the conlinin promoter and the other from a catalase-l gene in the antisense orientation. The inverted repeat was constructed using the vector system described by Helliwell and Waterhouse (2005).

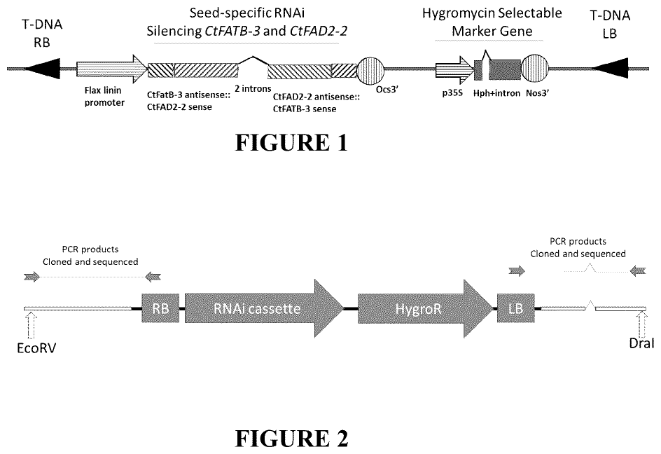

The DNA fragment containing the inverted repeat was inserted into a plant binary expression vector pORE-CBIb (Coutu et al., 2007) to generate the vector pCW732. The T-DNA of the vector contained a selectable marker gene that encodes hygromycin phosphotransferase (Hph, SEQ ID NO:8), thereby allowing selection for tolerance to hygromycin in tissue culture during the transformation process. The hph gene was expressed with the 35S promoter. FIG. 1 shows a schematic genetic map of the T-DNA region of pCW732. The nucleotide sequence of the T-DNA in pCW732 including the inverted repeat sequence is provided herein as SEQ ID NO:7. The sequence is annotated in the legend to SEQ ID NO: 7.

The genetic construct pCW732 was used to transform excised cotyledons and hypocotyls from plants of the safflower line CBI1582 using the Agrobacterium-mediated method with rescue of regenerated shoots using a grafting method (Belide et al., 2011). Ten independent, confirmed transformed shoots growing on non-transformed root-stocks (hereinafter termed To plants) were regenerated using the vector pCW732 and grown to maturity. Integration of the T-DNA in the To safflower scions was confirmed by PCR using T-DNA vector-specific primers as described by Belide et al. (2011). Seeds (T1 seeds) were harvested from the mature plants and assayed for increased oleic acid in the seedoil. At least six of the transformants yielded some seed having between 90% and 95% by weight oleic acid in the total fatty acid content of the seedoil as well as segregants having the same oleic acid content as CBI1582 and some intermediate phenotypes. Other transformants such as pCW722-82 did not yield seeds with at least 90% oleic acid, instead having approximately 80% oleic acid.

Various criteria were used to select the two best lines from the transformants over eight progeny generations, resulting in selection of progeny plants from the GOR73226 and GOR73240) lines as the optimal transgenic lines. The most important criterion was the fatty acid composition, namely an oleic acid level of >90% by weight, as well as a reduced palmitic acid level (<4.0%) and a greatly reduced linoleic acid (LA) level (<3.75%) compared to the parental line CBI1582. Other criteria used were the stability of the fatty acid trait over the generations, including in field trials, oil content in the seed, having single T-DNA insertions, agronomic performance and other traits as described in the following examples. To do this, T2 seed from transgenic plants that were homozygous for a single T-DNA insertion and having high oleic acid content were selected and propagated to produce T3 seed. These were again analysed non-destructively and the process repeated. T4 seed were sown in the field in 2014 near Kununurra, Western Australia under OGTR regulatory approval and the T4 plants evaluated for agronomic performance. T5 seed were planted near Narrabri, NSW in 2015 and the resultant plants evaluated. This process was repeated with subsequent progeny generations in multi-site field trials to the T9 generation.

### Example 3. Molecular Characterisation of Transgenic Lines

### Determination of the T-DNA Copy Number

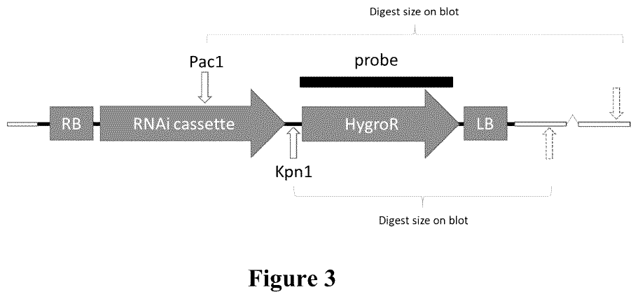

In order to analyse the number of T-DNA insertions and the insertions sites into the safflower genome for each transformed line, ten T4-generation plants of each of the transformed lines including GOR73226 and GOR73240 were grown in a glasshouse and DNA extracted from the combined plant material. The extracted DNAs were purified on caesium chloride gradients so that they were suitable for digestion with restriction enzymes, in this case KpnI and PacI, separately. Southern blot hybridisation analysis was conducted following the protocol according to Belide et al. (2011). In each case. 1 mg of DNA was digested overnight with KpnI or PacI (NEB. USA) according to the supplier's instructions. Both KpnI and PacI each digest at one site within the T-DNA fragment, but outside of the hygromycin phosphotransferase gene region (FIG. 3). The digested DNAs along with control DNA were electrophoresed on an agarose gel and blotted to a membrane by standard methods. For preparation of a radio-labelled probe. PCR primers were used to amplify the entire coding region of the Hph gene from pCW732. This amplicon was used as a template for a further round of PCR to generate a clean template for the probe free of contaminating vector backbone sequence. The probe was radiolabelled using random primer integration and radiolabelled ³²P-NTP ribonucleotides as previously described (Belide et al., 2011). After gel electrophoresis and blotting onto a membrane, the radiolabelled probe for the hygromycin phosphotransferase gene was hybridised to the membrane under stringent conditions and the membrane washed under stringent conditions.

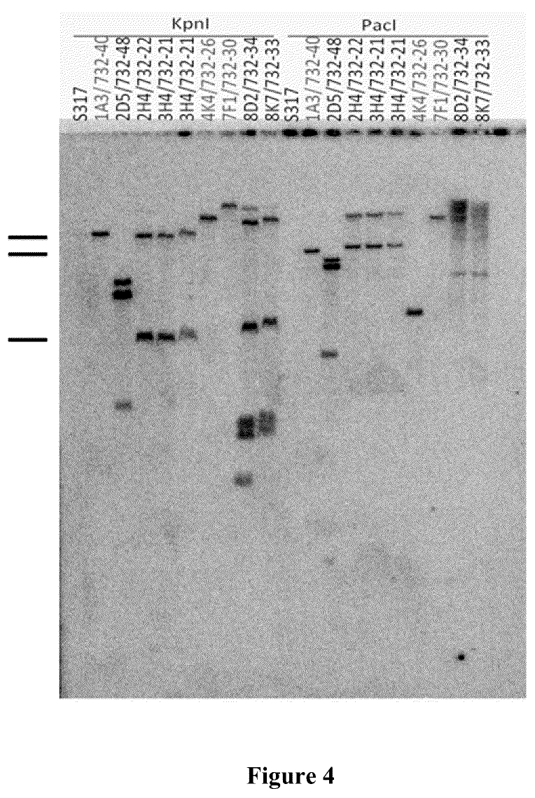

A photograph of the Southern blot is shown in FIG. 4. Only one hybridising band was observed for the GOR73226 (732-26) and GOR73240) (732-40) transformants for each of the PacI and KpnI enzymes, whereas some of the other transgenic plants analysed yielded two, three or up to six hybridising bands. It was concluded that the GOR73226 and GOR73240 plants each had only one T-DNA insertion into the genome. In contrast, the independent transgenic lines 21. 33 and 48 produced multiple bands, consistent with insertion of multiple T-DNA insertions within each of those transformants. The probe did not hybridise with the DNA from the non-transformed safflower plants, as a negative control.

### Insertion Sites and Flanking Sequences for the T-DNAs in GOR73226 and GOR73240)

In order to determine the insertion site for each of the GOR73226 and GOR72640 transgenic lines and isolate the junction sequences, the genomic DNA was used to map the precise location of the T-DNA insertion sites in the safflower genome. A Universal GenomeWalker™ 2.0 kit (Clontech) was used with the supplier's protocol to clone and identify the flanking sequences outside of the T-DNA Left and Right borders (FIG. 2). The protocol used digestion of the plant genomic DNAs with a restriction enzyme followed by adaptor ligation and PCR-based amplification to clone the flanking sequences. The oligonucleotide primer for each primer/adaptor pair was located just inside the Left Border or Right Border sequence of the T-DNA so that the sequences flanking the T-DNA were amplified, being the safflower genomic sequences adjacent to the T-DNA insertion sites. For the Left border cloning, the DNA was digested with DraI. For the Right Border cloning, the DNA was digested with EcoRV.

Amplicons from the genome walking analysis were cloned and their nucleotide sequences determined using standard techniques. Since the nucleotide sequence of the T-DNA from pCW732 was known, the junction sequence and therefore the flanking sequences could be readily identified. Using the GOR73226 DNA and the primer/adaptor pair for the region flanking the Left Border, only one amplicon of approximately 1000 bp long was cloned and sequenced (FIG. 5). The analysis using the primer/adaptor regions for the Right Border region also produced only one amplicon of approximately 1400 bp long. The safflower sequences flanking the Left and Right Border were determined from the sequences of the junction fragments. When aligned with a draft genome sequence of wild-type safflower (Bowers et al., 2016), which covered approximately 80% of the safflower genome with approximately 200.000 fragments/contigs, the sequences flanking the Left and Right Borders for GOR73226 matched to a single DNA contig in the draft genome. It was concluded that the T-DNA in GOR73226 had inserted into this contig, into a region of the contig having the sequence provided as SEQ ID NO:11. The combination of sequencing of the GOR73226 amplicons and the alignment to the wild-type safflower genome found that during the insertion of the T-DNA. 155 basepairs of the genomic DNA was deleted at the insertion site. Further, the analysis revealed that the entire Left Border sequence was inserted into the genome, but only 41 bp of the 162 bp Right Border sequence was inserted. Such rearrangements are commonly observed during integration of a T-DNA. The nucleotide sequences for the junction fragments for GOR73226 containing the Right and Left border sequences are provided as SEQ ID NO: 10 and SEQ ID NO:12, respectively. The nucleotide sequences of the Right and Left borders integrated into GOR73226 are provided as SEQ ID NO:9 and SEQ ID NO:13, respectively. The nucleotide sequence of a portion of the GOR73226 Right border junction is provided as SEQ ID NO: 14, and for the GOR73226 Left border junction as SEQ ID NO: 15. These sequences readily distinguish GOR73226 from other transgenic lines having insertions elsewhere in the genome. The nucleotide sequence spanning the T-DNA insertion including about 1 kb of flanking safflower genomic sequence upstream and downstream of the T-DNA is provided as SEQ ID NO:33.

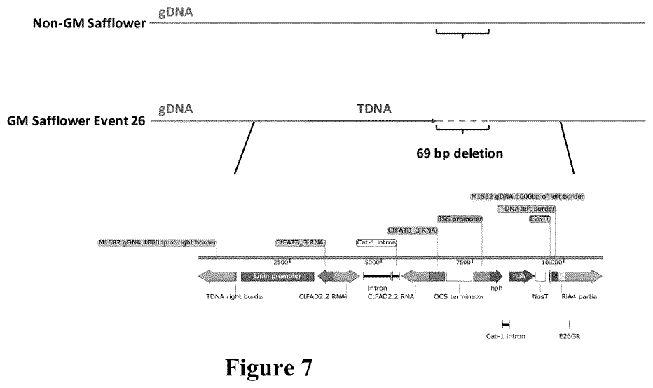

Based on this analysis a map of the pCW732 insertion into GOR73226 was generated (FIG. 7).

In analogous fashion, amplicons from the genome walking analysis for GOR73240 were cloned and their nucleotide sequences determined, using DNA from T4—and T7-generation plants. The junction sequences and therefore the flanking sequences from the safflower genome were readily identified. Using the GOR73240 DNA and the primer/adaptor pair for the region flanking the Left Border, only one amplicon of approximately 1000 bp long was cloned and sequenced (FIG. 5). The analysis using the primer/adaptor regions for the Right Border region also produced only one amplicon of approximately 6000 bp long. The safflower sequences flanking the Left and Right Borders were determined from the sequences of the junction fragments. When aligned with the draft genome sequence of wild-type safflower, the sequences flanking the Left and Right Borders for GOR73240 matched to a single DNA contig in the draft genome. It was concluded, as for GOR73226, that the T-DNA in GOR73240 had inserted into a region of this contig having the sequence provided as SEQ ID NO: 18. The combination of sequencing of the GOR73240 amplicons and the alignment to the wild-type safflower genome found that during the insertion of the T-DNA, the insertion of T-DNA generated a 34 bp deletion and 35 bp duplication within the genomic region (FIG. 6). Further, the analysis revealed that both the Left Border (13 bp) and Right Border (39 bp) sequences were truncated. The nucleotide sequences for the junction fragments for GOR73240 containing the Right and Left border sequences are provided as SEQ ID NO: 17 and SEQ ID NO:19, respectively. The nucleotide sequences of the Right and Left border sequences integrated into GOR73240) are provided as SEQ ID NO:16 and SEQ ID NO:20, respectively. The nucleotide sequence of a portion of the GOR73240 Right border junction is provided as SEQ ID NO:21, and for the GOR73240 Left border junction as SEQ ID NO:22. These sequences readily distinguish GOR73240 from other transgenic lines having insertions elsewhere in the genome. The nucleotide sequence spanning the T-DNA insertion including about 1 kb of flanking safflower genomic sequence upstream and downstream of the T-DNA is provided as SEQ ID NO:34.

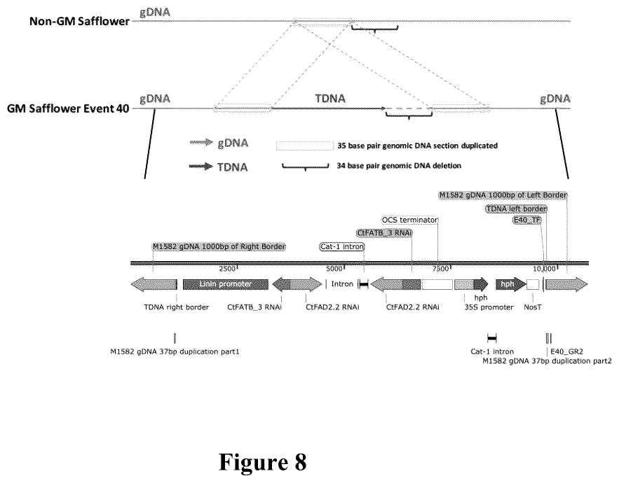

Based on this analysis a map of the pCW732 insertion into GOR73240 was generated (FIG. 8).

Conclusions from PCR Based Genome Walking

Overall, it was concluded from these results that GOR73226 and GOR73240 each contained a single-copy T-DNA insertion with no other partial or complete T-DNA components in the genome. Since the Left Border and Right Border sequence analyses provided the same results when using DNA from T7-generation plants as for the T4-generation plants, it was concluded that GOR73226 and GOR73240 had stably-inherited insertions into the safflower nuclear genome.

### Absence of the Vector Backbone Sequences in GOR73226 and GOR73240

Five pairs of PCR primers (Table 1) were designed across regions of the transformation vector pCW732 outside of the T-DNA, including regions of the bacterial origin of replication and the bacterial antibiotic selection marker encoding NptII. DNA isolated from T6-generation plants grown in the field under regulatory approval were used in PCR reactions with the primer pairs, using standard techniques. The PCR reaction products were electrophoresed on 1% agarose gels. DNA from the non-transgenic safflower variety S-317 was used as a negative control and plasmid DNA from the binary vector pCW732, diluted to the appropriate concentration and added to control DNA, was used as a positive control. No amplicons of the expected sizes were detected from the GOR73226 and GOR73240 DNAs. In addition, all of the control reactions demonstrating the presence or absence of target fragments as expected. It was concluded that no vector backbone sequences were integrated into the genomes of GOR73226 and GOR73240 plants during the transformation process.

TABLE 1
PCR primers to detect T-DNA vector backbone
sequence

| Primer | Sequence (5′ to 3′) | SEQ ID NO |
|---|---|---|
| P1FW | GTCGGCAAATCGTCAGACTT | 23 |
| P1RV | GTGGAGTCAGGCTTGATCGT | 24 |
| P2FW | TGTTCCCGGATCGAAGTAAG | 25 |
| P2RV | ACATCCTTGGCGTCTCAACT | 26 |
| P3FW | AATGTTCGAATGCCCTTCTC | 27 |
| P3RV | AGGGCGACCTCTTTTTGG | 28 |
| P4FW | GACAAGTGGTATGACATTGC | 29 |
| P4RV | CTAAAACAATTCATCCAG | 30 |
| P5FW | AGATCCTCTTCCGCTTCCTC | 31 |
| P5RV | TACCGGGTTGGACTCAAGAC | 32 |

### Example 4. Assessment of the GOR73226 and GOR73240 Oil Composition Phenotype

Fatty Acid Analysis of F2 Progeny from Transgenic Plants and from Crosses

The T2 and later generations of transgenic safflower seeds, and F2 seed populations from several crosses, were analysed for fatty acid composition. To do this, safflower seeds were imbibed on wet filter paper overnight at room temperature to allow the seed coat to be easily removed. The tip of a cotyledon from each imbibed seed, about 5 mm, was excised and sampled for fatty acid composition analysis by conversion of the fatty acids to fatty acid methyl esters (FAME) and quantitation by GC. To retain the seed, the remaining part of each seed was planted in soil in pots and resultant plants grown to maturity in a glasshouse.

FAME were prepared essentially as described by (Zhou et al., 2013), with slight modifications as follows. The methylation was extended to 4 hr using 800 μL 1N methanolic-HCl (Supelco, Bellefonte, USA). GC analysis with FID was also performed as described by (Zhou et al., 2013), except the ramping program was changed to an initial temperature at 150° C. holding for 1 min, then raised to 180° C., at 10° C./min, and to 240° C., at 50° C./min holding for 4 min. GLC standard 411 (Nuchek, Elysain, USA) was used for calibration.

Fatty acid analyses were also performed by the NSW Department of Primary Industries Oil Testing laboratory, a commercial testing laboratory registered with the National Association of Testing Authorities (NATA), utilising validated technical procedures and methods in accordance with industry standards.

The results for the fatty acid composition analysis for GOR73226 and GOR73240 seeds of the T7 generation from across 5 independent field trials conducted in 2016 are presented in Table 2. Values are ±standard error. Means with the same letter are not significantly different (>0.01). It was observed that GOR73226 and GOR73240 provided seedoil having consistently between 90% and 95% oleic acid with less than 4% palmitic acid and less than 2.75% linoleic acid. In contrast, the parental line CBI1582 had about 76% oleic acid and about 15% linoleic acid. All lines lacked α-linolenic acid (ALA). Standard errors were considered to be very low. The fatty acid composition was therefore remarkably stable from the T2 generation through to at least the T7 generation of seeds. Essentially the same results were observed in other field trials from geographically distinct environments. These data demonstrate the stability of the down-regulation of the CtFAD2-2 and CtFATB-3 genes by the RNAi construct to provide seedoil having 91-93% oleic acid.

TABLE 2
Fatty acid profile of GOR73226 and GOR73240 seedoil
compared to the parental line CBI1582.

| Fatty acid(s) | GOR73226 | GOR73240 | CBI1582 |
|---|---|---|---|
| Palmitic acid C16:0 | 3.46 ± 0.13^{a} | 3.53 ± 0.24^{a} | 6.27 ± 0.12^{b} |
| Palmitoleic C16:1 | 0.07 ± 0.04^{a} | 0.12 ± 0.07^{a} | 0.03 ± 0.01^{a} |
| Stearic acid C18:0 | 1.33 ± 0.12^{a} | 1.64 ± 0.12^{ab} | 1.76 ± 0.09^{a} |
| Oleic acid C18:1Δ9 | 92.50 ± 0.28^{a} | 91.82 ± 0.49^{a} | 75.91 ± 0.59^{b} |
| Linoleic acid C18:2Δ9, 12 | 2.52 ± 0.22^{a} | 2.65 ± 0.28^{a} | 15.73 ± 0.58^{b} |
| Linolenic acid C18:3Δ9, 12, 15 | 0.0^{a} | 0.0^{a} | 0.0^{a} |
| Arachidic acid C20:0 | 0.14 ± 0.03^{a} | 0.20 ± 0.04^{a} | 0.23 ± 0.03^{a} |

Crossing GOR73226 with a High Oleic-Type Non-Transgenic Safflower

An individual plant of GOR73226 of the T4 generation was grown in a glasshouse alongside a non-transgenic plant of variety S-317 having a high (about 75%) oleic acid genotype. During flowering, the two plants were manually crossed and all unused florets were emasculated to prevent self-pollination. This procedure generated 6 F1 seeds. The F1 seeds were sown and the resultant F1 plants grown to maturity. The F1 plants were allowed to self-pollinate, resulting in a population of 119 F2 seed. Each of the 119 seed were analysed for their fatty acid profile. The number of F2 seeds that contained levels of oleic acid greater than 90% by weight and palmitic acid levels less than 4% were evaluated, looking for a 3:1 segregation ratio of the high oleic acid trait. Of the 119 F2 seed produced from the cross, 37 seeds had palmitic acid levels greater than 4%. Of these, 36 individuals had oleic acid levels of less than 90%. These seeds were considered to be segregants lacking the T-DNA. Chi-square goodness of fit tests indicated that there was no significant departure from the predicted 3:1 segregation ratio (palmitic acid greater than 4% X²[1,N=119]=2.36, P=0.153, p<0.01: oleic acid levels less than 90% X²[1,N=119]=1.75, P=0.224, p<0.01). Based on this analysis, the insertion of the T-DNA in GOR73226 was stably inherited and segregated in a 3:1 ratio in accordance with Mendelian inheritance of a dominant genetic locus.

Crossing GOR73240 with a High Oleic-Type Non-Transgenic Safflower

In a similar fashion, an individual plant of GOR73240 of the T4 generation was grown in a glasshouse alongside a non-transgenic plant of the variety Montola 2003 having a high oleic acid genotype. The two plants were manually crossed and all unused florets were emasculated to prevent self-pollination. The F1 seed were sown and the F1 plants grown to maturity, producing a population of 59 F2 seed. Each of the 59 seed were analysed for their fatty acid profile. Of the 59 F2 seed, 14 had palmitic acid levels greater than 4% and oleic acid levels equal to or less than 90%, representing segregants lacking the T-DNA. Chi-square goodness of fit tests indicated that there was no significant departure from the predicted 3:1 segregation ratio (palmitic acid greater than 4% X²|1,N=59]=0.05, P=0.822, p<0.01: Oleic acid levels less than 90% X²[1,N=59]=0.277, P=0.0.599, p<0.01). Based on this analysis, the insertion of the T-DNA in GOR73226 was stably inherited and segregated in a 3:1 ratio in accordance with Mendelian inheritance of a dominant genetic locus.

Crossing GOR73240 with a High Linoleic-Type Non-Transgenic Safflower

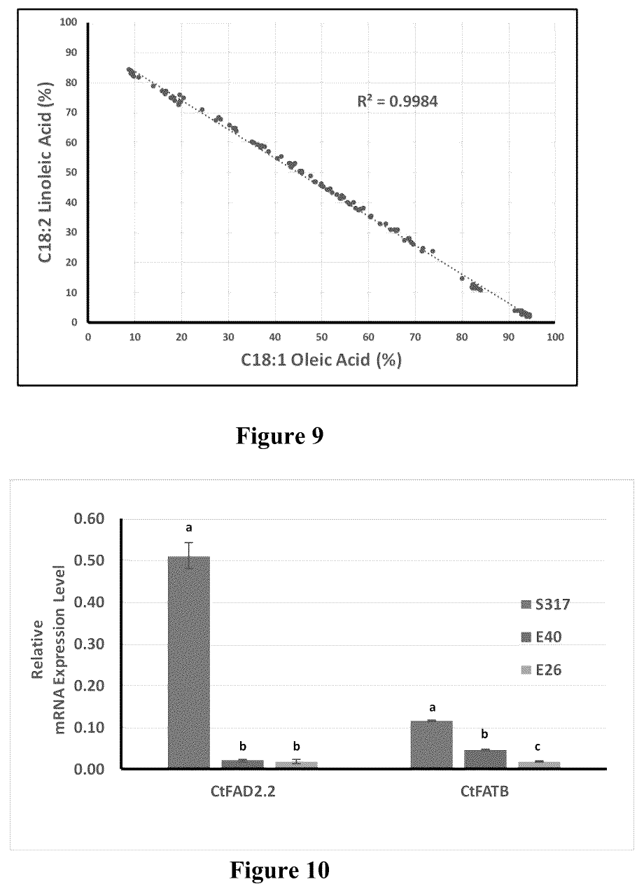

Further, an individual plant of GOR73240 of the T4 generation was grown in a glasshouse alongside a non-transgenic plant having a high linoleic acid (>70%) phenotype, and the plants crossed. The F1 seed were sown and the resultant F1 plants grown to maturity with self-pollination to produce a population of 126 F2 seed. Each of the 126 seed were analysed for their fatty acid profile. The number of F2 seeds that contained levels of oleic acid greater than 90% and the number of seeds with linoleic levels greater than 70% were evaluated with respect to a 3:1 segregation ratio. As expected from a cross between a linoleic type and a high oleic type, levels of linoleic acid were negatively correlated with oleic acid (FIG. 9). Of the 126 F2 seeds, 27 seeds had oleic acid levels greater than 90%. These plants contained a functional T-DNA i.e. had super high oleic acid levels. Chi-square goodness of fit tests indicated that there was no significant departure from the predicted 3: 1 segregation ratio (Oleic acid greater than 90% X²[1,N=126]=0.68, P=0.4096, p<0.01). Of the 126 F2 seeds, 24 seeds had linoleic acid levels greater than 70%. These seeds did not contain the T-DNA and were similar to the high linoleic acid parental line. Chi-square goodness of fit tests indicated that there was no significant departure from the predicted 3:1 segregation ratio (linoleic acid greater than 70% X²[1,N=126]=2.08, P=0.1492, p<0.01). Based on this analysis, the insertion of the T-DNA in GOR73226 was stably inherited and segregated in a 3:1 ratio in accordance with Mendelian inheritance of a dominant genetic locus.

### Example 5. Lipid Analysis in GOR73226 and GOR73240 Plants

### Profiling Membrane-Associated Lipid Species at Different Plant Developmental Stages

Total lipids were extracted from freeze-dried cotyledon, hypocotyl, roots and true leaves of two-week-old safflower plants of different varieties or lines. The varieties and lines were: GOR73226 and GOR73240 (SHO), high oleic non-transgenic varieties S-317 (HO1) and Lesaf496 (HO2), a high oleic non-transgenic safflower variety developed by EMS mutagenesis (ems/S901) that had compromised yield (U.S. Pat. No. 5,912,416), and a wild-type, low oleic acid safflower variety Centennial (LO). Freeze-dried leaf tissue from plants of each variety or line was ground to a powder in a micro-centrifuge tube containing a metallic ball using a Reicht tissue lyser (Qiagen) for 3 min at a frequency of 20 per sec. Chloroform:methanol (2:1, v/v) was added and mixed with the powder for a further 3 min using the tissue lyser before the addition of 1:3 (v/v) of 0.1 M KCl. The sample was then mixed for a further 3 min before centrifugation (5 min at 14,000 g), after which the lower lipid phase was collected. The remaining aqueous phase and cell debris was washed once with chloroform, centrifuged, and the lower phase removed and pooled with the earlier extract. The lipid phase solvent was then evaporated completely using nitrogen gas flow and the extracted lipid re-suspended using 1 ml chloroform per 20 mg extracted lipid.

Lipidomics Analysis Via LC-MS Lipids extracts were diluted in 1:100 mL butanol:methanol (1:1, v/v) and analyzed by liquid chromatography-mass spectrometry (LC-MS), based on previously described methods (Reynolds et al., 2015). Briefly, an Agilent 1290 series LC and 6490 triple quadrupole LC-MS was used with Jet Stream ionisation. The phosphatidylcholine (PC) and lysophosphatidylcholine (LPC) species were separated on an Agilent 120 HILIC column (2.1×100 mm. 2.7 μm), over a gradient from 95% acetonitrile to 75% acetonitrile with 20 mM ammonium acetate. PC and LPC hydrogen adducts were quantified by the characteristic 184 m/z phosphatidyl head group ion The 5 under positive ionisation mode, ammonium adducts of monogalactosyldiacylglycerol (MGDG), digalactosyldiacylglycerol (DGDG), diacylglycerol (DAG) and TAG lipid species were analyzed by the neutral loss of singular fatty acids C16 to C20. Multiple reaction monitoring (MRM) lists were based on the following major fatty acids: 16:0, 16:3, 18:0, 18:1, 18:2, 18:3, using a collision 30 energy of 28 V. Lipids were chromatographically separated using an Agilent Poroshell column (50 mm×2.1 mm. 2.7 μm) and a binary gradient with a flow rate of 0.2 mL/min. The mobile phases were: A. 10 mM ammonium formate in H₂O:acetonitrile; isopropanol (5:45:50, v/v): B. 10 mM ammonium formate in H2O:acetonitrile; isopropanol (5:20:75, v/v). Individual MRM TAG was identified based on ammoniated precursor ion and product ion from neutral loss. Results were integrated using Agilent Mass Hunter Quantitative software and exported into R for statistical and graphical analysis.

The LC-MS method provided a comprehensive analysis of lipids in seed and non-seed tissues in GOR73226 and GOR73240 plants compared to high oleic and low oleic safflower varieties. In roots and true leaves, the dominant lipid species such as diacylglycerol (DAG), digalactosyldiacylglyercol (DGDG) and monogalactosyldiacylglyercol (MGDG) were the same in GOR73226 and GOR73240 compared to the LO. HO1 and HO2 plants, each exhibiting the high polyunsaturated fatty acid composition typical of those vegetative tissues. In contrast, ems/S901 plants contained a marked increase in the monounsaturated content relative to the polysaturated fatty acids. These results indicated that the RNAi transgene encoded by the T-DNA of pCW732 used in generating GOR73226 and GOR73240 was not having an effect in non-seed tissues and was restricted to seed and developmentally-derived organs, such as the emergent cotyledons and hypocotyls.

### Example 6. RNA Analysis of the FAD2 and FATB Genes in the Transgenic Plants

### Down-Regulation of CtFATB and CtFAD2.2 Genes in GOR73226 and GOR73240 Plants

To analyse RNA expression in the transgenic plants. GOR73226. GOR73240) and non-transgenic control plants were grown in a glasshouse until flowering. Florets were manually self-pollinated to set the time of seed development and developing embryos were sampled for total RNA 15 days after pollination. Total RNA was extracted from the developing safflower seed. For RT-PCR analysis of gene expression, the precipitated RNA was further purified to remove small RNAs by using Plant RNAeasy columns (Qiagen) according to the supplier's instructions except that the chloroform extraction was repeated and the RNA precipitated overnight at −20° C. RQ1 DNAse (Promega) was used to remove contaminating DNA, cDNA synthesis was carried out using Superscript III reverse transcriptase (Life Technologies. Invitrogen) according to the manufacturer's protocol with an oligo dT primer (Invitrogen). For each RNA sample, three separate cDNA synthesis reactions were carried out. Real-time quantitative (qRT)-PCR was carried out as described (Allen et al., 2007). Data analysis was performed using SPSS Statistics (version 23) with the significance of differences between means tested using the Least Significant Difference (LSD) test (p<0.05).

Using these methods to prepare the RNA samples, quantitative PCR was used to measure the abundance of RNA expressed from the CtFAD2-2 and CtFATB-3 genes in GOR73226 and GOR73240 plants relative to the non-transgenic high-oleic safflower variety S-317. The expression levels of CtFATB-3 and CtFAD2-2 were significantly reduced (p<0).05) in GOR73226 and GOR73240 compared to safflower S-317 (FIG. 10). For CtFATB-3. GOR73226 and GOR73240 were significantly different (p<0.01) from each as well as from S-317. They were not significantly different (p>0.05) in CtFAD)2-2 expression, both being greatly reduced compared to S-317.

Small RNA Analysis Total RNA isolated from the plants was subjected to deep sequencing using the Illumina TruSeq small RNA Sample Prep Kit and Illumina based 100 bp single read technologies (John Curtin School of Medical Research, Canberra, Australia). The sRNA sequence database was trimmed of adaptor sequences using Trimmimatic (Bolger et al. 2015) and were back-aligned to template sequences of (IFAD2.2 and CtFATB using ShortStack (Axtell 2013), allowing a single mismatch. Locations with a minimum unique read coverage of 5 are reported.

The populations of small RNA molecules, generally in the size range 21-24 nt, extracted from GOR73226 and GOR73240 were sequenced and mapped against the draft safflower genome sequence. The only locations where these small RNA populations mapped were within the transcribed regions of the CtFAD2-2 and CtFATB-3 genes. In particular, the regions where the small RNAs aligned were mostly confined to the gene regions that were used in the design of the hairpin RNA gene in pCW732 (SEQ ID NOs:5 and 6). A wider examination of small RNAs aligning elsewhere in the safflower genome failed to find any significant hits, defined here as above a threshold of 10 hits across an open reading frame (ORF).

It was concluded from the lipidomics and RNA analyses that the CtFAD2-2 and CtFATB-3 genes were specifically down-regulated in the developing seed of the safflower plants, mediated by the small RNA molecules derived from the hairpin RNA, that mapped to the CtFATB-3 and CtFAD2-2 genes. This was consistent with an RNAi-mediated mechanism of reduction in gene expression. There was no down-regulation of the genes in the plant tissues other than the seed.

### Example 7. Expression of the Hph Gene in GOR73226 and GOR73240 Plants

To test for expression of the Hph gene in the transgenic plants, seeds from field grown GOR73226, GOR73240 plants of the T8 generation and the non-transgenic parental line CBI1582 were sown in soil in pots. Samples of true leaves having a 2 cm diameter were harvested after 3 weeks growth. Western blot analysis was performed on protein extracts as described by Belide et al., (2011). Briefly, leaf material was ground in liquid nitrogen to a powder. Approximately 5 mg of powder was added to 300 μL of standard Laemmli Buffer, heated to 95° C. for 5 minutes, cooled to room temperature, and centrifuged at 10000 rpm for 5 min. 20 μL of each supernatant was applied to a denaturing SDS gel having a 4-12% polyacrylamide gradient (Life Technologies). Proteins were electrophoresed at 200 mA for 40 min, using a BenchMark protein ladder as a standard for sizing. The proteins from the gels were blotted onto a prepared PVDF membrane and probed using a primary mouse monoclonal antibody raised against Hph protein (HYHmb: mybioSource.com product MBS857772: 1:2000 dilution) and a secondary antibody (anti-mouse HRP: 1:5000). Positive controls to Hph (F11576 and F11772 plants) were included on all membranes. F11772 was an F1 hybrid plant between GOR73240) and a non-transgenic safflower, where all alleles are expected to be hemizygous. F11576 was another safflower plant transformed with pCW732 (Event 33), containing about 5 T-DNA copies.

The Western blot analysis of GOR73226 and GOR73240 revealed a single polypeptide band of about 40 kD that bound the antibody, corresponding to the Hph protein. There was no evidence of multiple protein products and no Hph protein was detected in the non-transgenic safflower CBI1582. The protein expression profiles were essentially the same between plants of GOR73226 and GOR73240.

The HPH protein catalyses the phosphorylation of the 4-hydroxyl group of the antibiotic Hygromycin B, rendering it inactive. This is highly specific for a limited number of antibiotics that are not used for human clinical applications and has no effect on aminocyclitol or aminoglycoside antibiotics. In animal studies, the protein has no acute toxicity and database analysis reveals no similarity to known toxic proteins or allergens. The protein is not glycosylated in plants and the protein is rapidly degraded in gastric fluid.

### Example 8—Summary Agronomic Assessment of GOR73226 and GOR73240 Field Trials

Field trials were conducted for the purpose of phenotypic and agronomic assessment and to provide seed and vegetative tissue samples for molecular characterisation and compositional analysis (Table 3). Plants of GOR73226. GOR73240, their parental line CBI1582 and conventional non-transformed safflower were grown across several growing regions in Australia under a licence issued by the Office of the Gene Technology Regulator.

The agronomic practices and pest control measures used were location-specific and were typical for all aspects of safflower cultivation and included soil preparation, fertilizer application, irrigation, and pesticide application. The field trials were established in a randomised complete block (RCB) design. At each site, the trials included the test, control, and reference varieties. In 2015, GOR73226 and GOR73240 seed were from the T4 generation and in 2016 from the T5 generation. Every block (replicate) included a plot of each treatment. The experimental unit was the plot. All plots within each block were independently randomised so that the treatments were in random order. Typically there were four replicates at each site. Within each replicate, each safflower variety was planted in plots arranged in random order.

Although some trials occurred in the same State, they were not planted in the same location. Plots were in different fields, or in different locations on the farm due to crop rotation practices. Field conditions such as environment, field history, soil type, pest presence, and drainage can differ from year to year.

### Assessment Methods

GOR73226 and GOR73240 characteristics (Table 4) were compared to the parental line CBI1582. Data analysis was performed using SPSS Statistics (version 23) with the significance of differences between means tested using the Least Significant Difference (LSD) test (p<0.01). A Levene's Test was performed to verify homogeneity of the variances (p>0.05) and where required, a data transformation was performed to normalise data and obtain homogeneity of the variances.

### Summary of Growing Conditions

Field trials were undertaken over two growing seasons in New South Wales and Victoria. The growing conditions in each State and over each year were substantially different. A summary of growing season conditions was provided in 2015 and 2016 Crop Reports published by the Australian Bureau of Agricultural and Resource Economics and Sciences, summarised as follows.

In Victoria in 2015, the crop growing season was mixed. The safflower sites located in Victoria had a poor start with below average rainfall with seasonal conditions remaining unfavourable over the major cropping regions. Winter rainfall was significantly below average, particularly in August, and soil moisture levels are well below average. Rainfall was sufficient for crops to continue developing, but yield prospects declined. Rainfall in Victoria was below average during September and particularly October and was accompanied by unusually high daytime temperatures in early October. The extent to which yield potential of individual crops was adversely affected depended on stage of crop development and regional differences in the condition of crops at the beginning of spring. The unfavourable seasonal conditions also adversely affected crop quality. Overall, the safflower sites in Victoria were stressed.

TABLE 3
Field trial sites used for agronomic assessment

| Trial Site | | Tria | Date | Flowering | Harvest |
|---|---|---|---|---|---|
| Number | Location | design | Planted | Date* | Date |
| DIR131 | West | 0.233 ha; | 10 Jul. | 30 Oct. | 12 Jan. |
| Site 1 | Wimmera | RCB, 4 | 2015 | 2015 | 2016 |
| (KA15-838) | (Kaniva), | replicates | | | |
| | Victoria | | | | |
| DIR131 | Horsham | 0.233 ha; | 10 Jul. | 30 Oct. | 12 Jan. |
| Site 2 | (Kalkee), | RCB, 4 | 2015 | 2015 | 2016 |
| (KA15-837) | Victoria | replicates | | | |
| DIR131 | Narrabri | 0.35 ha; | 21 Jul. | 30 Oct. | 2 Jan. |
| Site 3 | (Bellata), | RCB, 4 | 2015 | 2015 | 2016 |
| (KA15-834) | NSW | replicates | | | |
| DIR131 | Narrabri, | 0.37 ha; | 21 Jul. | 3 Nov. | 8 Dec. |
| Site 4 | NSW | RCB, 4 | 2015 | 2015 | 2015 |
| (KA15-833) | | replicates | | | |
| DIR131 | Horsham | 0.10 ha; | 28 Jun. | 14 Dec. | 14 Feb. |
| Site 8 | (Kalkee), | RCB, 4 | 2016 | 2016 | 2017 |
| (KA16-0947) | Victoria | replicates | | | |
| DIR131 | West | 0.10 ha; | 28 Jun. | 10 Dec. | 16 Feb. |
| Site 9 | Wimmera | RCB, 4 | 2016 | 2016 | 2017 |
| (KA16-0948) | (Kaniva), | replicates | | | |
| | Victoria | | | | |
| DIR131 | Narrabri | 0.25 ha; | 4 Jul. | 8 Nov. | 21 Dec. |
| Site 10 | (Bellata), | RCB, 4 | 2016 | 2016 | 2016 |
| (KA16-0937) | NSW | replicates | | | |
| DIR131 | Narrabri, | 0.75 ha; | 28 Jul. | 22 Nov. | 5 Jan. |
| Site 12 | NSW | RCB, 4 | 2016 | 2016 | 2017 |
| (KA16-0936) | | replicates | | | |
| DIR131 | Narrabri | 0.40 ha; | 29 Jul. | 14 Nov. | 4 Jan. |
| Site 13 | (Wee Waa), | RCB, 4 | 2016 | 2016 | 2017 |
| (KA16-0938 | NSW | replicates | | | |

*Flowering date refers to the date of the first flower for DIR131 reporting purposes

TABLE 4
Agronomic characteristics assessed

| Assessment | | |
|---|---|---|
| Type | Scale | Method |
| Vigour | % | 0 = No vigour, 9 = Most vigorous plot |
| Height | cm | Height of 10 plants were measured and the average taken |
| Flowering | days | Number of days to 50% flowering |
| Disease | 0-9 | 10 leaves were assessed and number of diseased leaves |
| Incidence | | were counted |
| Insect Infestation | 0-9 | 10 leaves were assessed for insect damage and counted |
| Lodging | 0-9 | The crop was assessed for lodging where 9 = no crop |
| | | lodging and 0 = completely flat |
| Shattering | 0-9 | The crop was assessed for shattering where 9 = no crop |
| | | shattering and 0 = 100% crop shattering and seed on the |
| | | ground |
| Yield | T/ha | Yield was measured with a small plot harvester and |
| | | extrapolated to a T/ha basis. |

In contrast, in 2015, NSW sites received average to above average rainfall. Seasonal conditions over September and October were generally hotter and drier than average in NSW cropping regions. Towards the end of October, and for much of November, heavy rainfall improved levels of upper layer soil moisture. Overall, the conditions in NSW were more favourable than Victoria.

Seasonal conditions in 2016 in the major cropping regions in Victoria were very favourable for crop development during the Spring. Spring rainfall was above average, which increased soil moisture levels, and temperatures were cooler than average. Seasonal conditions through Spring and into harvest were very favourable. However, waterlogging and lodging (fallen crops) adversely affected crops in some regions.

Many cropping regions in NSW had above-average rainfall in September 2016, after a wetter than average Winter in most parts of the state. The winter and early spring rainfall increased soil moisture levels, which resulted in record high yields in many regions. However, in some areas, the above average rainfall resulted in flooding and waterlogged crops, particularly in central and southern NSW. Seasonal conditions during November and December were not favourable with below average rainfall combined with above average temperatures depleted upper layer soil moisture leading to a hot finish for the safflower trials.

### Assessment of Seedling Vigour

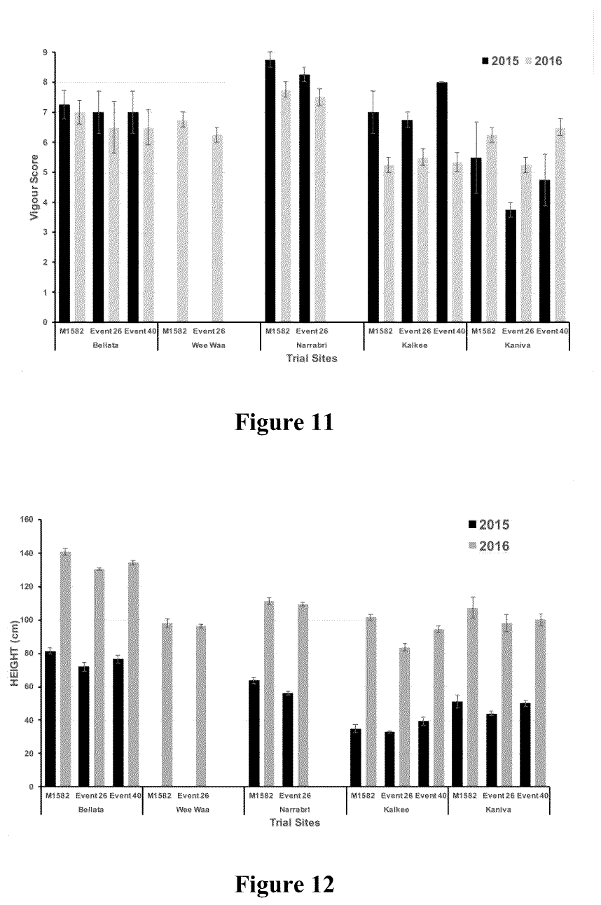

Seedling vigour is a characteristic that determines the potential for rapid and uniform seedling emergence and establishment of crops. The characteristic defines the initial growth and the ultimate yield potential of a crop. Each replicate trial plot at each trial site was assessed for seedling vigour at approximately 40 days after planting (FIG. 11).

In 2015, there was significant variation in seedling vigour between the trial sites (F_{[3.48]}=20.17: p<0.01), reflecting the differences in seasonal variation. However, there was no significant difference between GOR73226, GOR73240 and the parental line CBI1582 (F_{[2.48]}=1.33: p=0.28) and there was no significant interaction effect between safflower line or trial site (F_{[6.48]}=0.57: p=0.75).

Similarly, in 2016 there was significant variation in vigour between the trial sites (F_{[4.52]}=10.54: p<0.01), also reflecting the differences in seasonal variation. Similar to 2015, there was no significant difference between the lines tested (F_{[2.52]}=1.44: p=0.25) or interaction effect between the lines and trial sites (F_{[8.52]}=1.38; p=0.25).

The results from 2015 and 2016 over 9 different trial sites indicate that seedling vigour of the GOR73226 and GOR73240 safflower lines were not significantly different to the parental line CBI1582.

### Assessment of Plant Height

Each trial plot at every trial site was assessed for plant height at approximately 120 days after planting (FIG. 12). In 2015, there was significant variation in plant height between the trial sites (F_{[3.48]}=198.78: p<0.01), reflecting the differences in seasonal variation. There was also a significant difference in plant height amongst the safflower lines (F_{[2.48]}=9.93: p<0.01) with GOR73226 consistently shorter than GOR73240 and the parental control CBI1582. There was no significant interaction between trial sites and the safflower lines (F_{[6.48]}=1.21: p=0.33).

Similar observations were recorded in 2016 with significant differences between sites (F_{[4.52]}=91.72: p<0.01) and safflower lines (F_{[2.52]}=9.82: p<0.01), but no interaction between the height of safflower lines and site (F[8,52]=1.91: p=0.10). GOR73240 was no different to the parental line CBI1582, but GOR73226 was slightly shorter than both GOR73240 and the parental line at Bellata and Kalkee.

The results from 2015 and 2016 over 9 different trial sites indicate that plant height of the GOR73226 and GOR73240 safflower lines were not significantly different to the parental line CBI1582, although under some environments GOR73226 was slightly shorter than the other two.

### Time to Flowering

The time to flowering in safflower is highly dependent on variety and the time of sowing. To investigate if GOR73226 and GOR73240 had the same flowering time as the parental lime CBI1582, the time (number of days) to 50% flowering in an individual plot was assessed.

In 2015, two sites located in New South Wales were evaluated (Site 3: Narrabri and Site 4: Bellata). Although both trial sites were planted on the same day (21st July 2015), there was a significant difference between the trial sites (F_{[1.24]}=30.74: p<0.01) in terms of the time to flowering with Bellata (110 days) taking on average 4 days longer to flower than the Narrabri site (106 days). There was also a significant effect of variety across the sites (F_{[2.24]}=6.25: p<0.01), but no significant interaction effect (F_{[2.24]}=1.53: p=0.25). On average, GOR73226 (106.5+0.65 days) flowered slightly earlier than the parental line (109.75+0.65 days). GOR73240 (108+0.65 days) was not significantly different to either GOR73226 or the parental control.

In 2016, three sites in New South Wales were evaluated (Site 12: Narrabri, Site 10: Bellata and Site 13: Wee Waa). As seen in 2015, there was a significant effect (F_{[2.28]}=1182.72: p<0.01) of trial site on the time to flowering. This could be attributed, in part, to differences in planting dates between the sites. The site at Bellata was planted earlier and had the longest time to flowering (137+0.25 days) compared to Narrabri and Wee Waa (125+0.31 days and 116+0.31 days).

In contrast, over all the sites there was no significant difference between varieties (F_{[2.28]}=0.25: p=0.78) or any interaction between the varieties (F_{[2.28]}=0.50; p=0.61) and the trial sites.

In 2015, two sites located in Victoria were evaluated (Site 1: Kaniva and Site 2; Kalkee). Although both trial sites were planted on the same day (10th July 2015), there was a significant difference between the trial sites (F_{[1.24]}=9.99: p<0.01) in terms of the time to flowering with Kalkee (138 days) taking on average 3 days longer to flower than the Kaniva site (135 days). There was no significant effect of variety across the sites (F_{[2.24]}=4.80: p=0.02) or interaction effect (F_{[2.24]}=1.68: p=0.22). On average, GOR73226 (135+0.75 days) flowered slightly earlier than the parental line (138+0.75 days) and GOR73240 (137+0.75 days).

In 2016 in Victoria, there was no significant difference between the trial sites (F_{[2.24]}=1.42: p=0.25) or between varieties (F_{[2.24]}=3.32: p=0.06). The time to flowering for the parental control was 172+0.2 days, for GOR73226 173+0.2 days and for GOR73240, 172+0.2 days.

In summary, the assessment over two years and multiple sites indicated that GOR73226 and GOR73240 were not significantly different to the parental line CBI1582 in terms of the time to flowering.

### Assessment of Disease Incidence

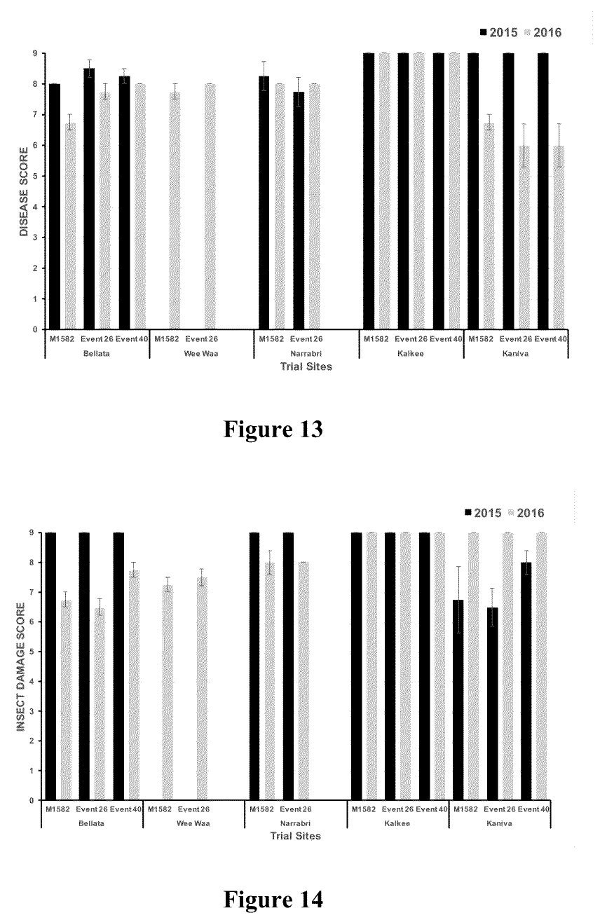

Each replicate trial plot at each trial site was assessed for disease incidence at approximately 120 days after planting (FIG. 13). In 2015, there was significant variation in disease incidence between the trial sites (F_{[3.48]}=12.49; p<0.01), reflecting the differences in seasonal variation. However, there was no significant difference between GOR73226, GOR73240 and the parental line CBI1582 (F_{[2.48]}=0.108: p=0.90) and there was no significant interaction effect between safflower line or trial site (F_{[6.48]}=0.85: p=0.54).

The same observations were recorded in 2016 with significant variation in disease incidence between the trial sites (F_{[4.52]}-23.97: p<0.01) and no significant difference between GOR73226, GOR73240 and the parental line CBI1582 (F_{[2.52]}=0.15: p=0.87) and there was no significant interaction effect between safflower line or trial site (F_{[8.52]}=1.85: p=0.11).

In summary, the results from 2015 and 2016 over 9 different trial sites indicated that disease incidence of the GOR73226 and GOR73240 SHO safflower lines were not significantly different to the parental line CBI1582.

### Assessment of Insect Damage

Each replicate trial plot at each trial site was assessed for insect damage at approximately 120 days after planting (FIG. 14). In 2015, there was significant variation in insect damage between the trial sites (F_{[3.48]}=16.03: p<0.01), reflecting the differences in seasonal variation. However, there was no significant difference between GOR73226, GOR73240 or the parental line CBI1582 (F_{[2,48]}=1.05: p=0.36) and there was no significant interaction effect between safflower line or trial site (F_{[6,48]}=1.05; p=0.41).

The same observations were recorded in 2016 with significant variation in insect damage between the trial sites (F_{[4.52]}-52.33: p<0.01) and no significant difference between GOR73226, GOR73240 and the parental line CBI1582 (F_{[2.52]}=3.60; p=0.04) and there was no significant interaction effect between safflower line or trial site (F_{[8.52]}=2.67: p=0.03).

In summary, the results from 2015 and 2016 over 9 different trial sites indicated that the incidence of insect damage in the transgenic safflower lines were not significantly different to the parental line.

### Assessment of Harvest Lodging

Each replicate trial plot at each trial site was assessed for harvest lodging at approximately 165 days after planting and capsule shattering. In 2015, there was no significant variation in lodging between the trial sites (F_{[3.48]}=2.64: p=0.06). Similarly, there was no significant difference between GOR73226, GOR73240 and the parental line (F_{[2.48]}=0.81: p=0.84) and there was no significant interaction effect between safflower line or trial site (F_{[6.48]}=0.81: p=0.98). In 2016, there was significant variation in lodging between the trial sites (F_{[4.52]}=32.13: p<0.01) but no significant difference between GOR73226, GOR73240 or the parental line CBI1582 (F_{[2.52]}=0.30: p=0.74) and there was no significant interaction effect between safflower line or trial site (F_{[8.52]}=0.33: p=0.92). There was no pod shattering observed in any safflower plots over all sites and years.

### Yield Assessment

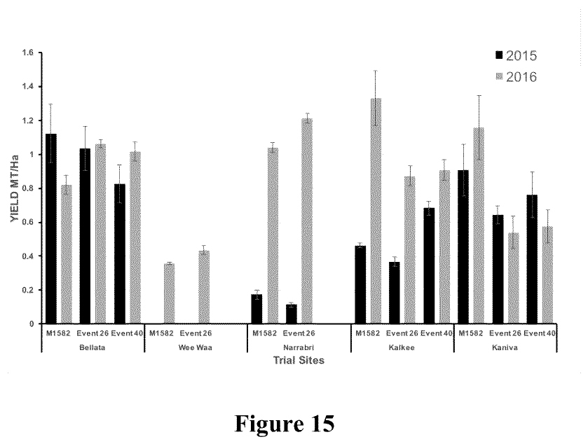

In 2015, there was significant variation in yield between the trial sites (F_{[3.48]}=1.84: p<0.01), reflecting the differences in seasonal and geographic variation (FIG. 15). However, there was no significant difference between GOR73226, GOR73240 or the parental line CBI1582 (F_{[2.48]}=4.10: p=0.25) and there was no significant interaction effect between safflower line or trial site (F_{[6.48]}=2.93: p=0.02). Similarly, in 2016 there was significant variation in yield between the trial sites (F_{[4.52]}=25.38: p<0.01), also reflecting the differences in seasonal and geographic variation. There was no significant difference between the safflower lines tested (F_{[2.52]}=4.66: p=0.02), however there was a significant interaction effect between the lines and trial sites (F_{[8.52]}=7.96: p<0.01). In NSW GOR73226 and GOR73240 yielded higher than the parental control, however the opposite was the case in Victoria.

Overall, the grain yield of the transgenic safflower lines was not significantly different to the parental line, with yield differences observed in 2015 and 2016 over 9 different trial sites associated with seasonal variability and geographic location rather than the presence of the transgenes. This was a very important criterion for selecting GOR73226 and GOR73240 rather than the other transgenic lines that had been created, in combination with the stability of the fatty acid composition of the seedoil over multiple trials in the field as described below.

### Conclusions

The agronomic assessment of the transgenic safflower lines GOR73226 and GOR73240 indicated that they were not significantly different to their parental control line in all of the assessed phenotypes. No adverse effects of the genetic modification were observed.

### Example 9. Compositional Assessment of GOR73226 and GOR73240 Safflower Seed

### Selection of Control and Reference Varieties

Safflower seed was chosen as the primary test material for compositional analysis of GOR73226 and GOR73240 because the seedmeal and oil fractions are derived from seed. The composition of seed was considered to be representative of these derived materials. The composition of vegetative tissue from field grown GOR73226 and GOR73240 was also examined. The most relevant comparator for these was the parental line CBI1582. Conventional non-transformed safflower varieties have a history of safe use for food and feed and were also used as reference varieties. Both oleic type and linoleic type safflower varieties were included. Such varieties are commonly used in bird seed and vegetable oil markets. The following reference varieties were included to provide a range of values common to conventional non-transformed safflower: Sironaria and Centennial (linoleic acid types), S-317, Montola 2003 and S901 (oleic acid types).

Several safflower seed products can be used as animal feeds: the seeds, the by-product of oil extraction (safflower meal) and the hulls, mostly used as a protein ingredient for animal feeding (Oelke et al., 1992). Safflower seeds used for oil production may be either cold pressed, expeller-pressed or solvent extracted. The by-products, safflower meal, and early stage vegetative tissue may be used for animal feeding. As such, feed quality assessments were undertaken on GOR73226 and GOR73240 seed meal derived from an expeller press and field grown vegetative tissue.

Seed and vegetative tissue samples were obtained from plants from some of the field trails described in Example 8, planted in RCB design. At least two geographically distant sites were chosen for seed analysis, Kalkee in Victoria and Bellata in New South Wales, with replicates pooled to provide a composite sample for analysis. Seed from the Bellata trial were also crushed to produce seed meal for feed testing. Oil samples for testing were obtained from seeds obtained from a field trial undertaken in 2014. Samples from four independent events were analysed and compared to the parental CBI1582 control and conventional non-transformed safflower varieties. Vegetative plant samples were also obtained from 38 day old plants from block plantings undertaken in Kununurra, Western Australia.

At each field trial site, seed was harvested from individual plots and kept separate. For the purposes of compositional analysis, replicates of each variety and transgenic line were pooled to form a composite sample.

Composite seed samples were processed by grinding prior to being analysed. This was undertaken following the protocols provided by each of the testing laboratories. All methods were undertaken by commercial testing laboratories utilising technical procedures and methods in accordance with industry standards.

A summary of analytes tested can be found in Table 5. These analytes were selected by considering the important nutritional components of safflower.

Data analysis was performed using SPSS Statistics (version 23) with the significance of differences between means tested using the Least Significant Difference (LSD) test (p<0.01). A Levene's Test was performed to verify homogeneity of the variances (p>0.05) and where required, a LOG (base 10) transformation was performed on proportional data to normalise data and obtain homogeneity of the variances.

### Analysis of Safflower Seed

Nutritional analysis was conducted on safflower seed to confirm that the composition of GOR73226 and GOR73240 remained within the normal levels for safflower when compared to the parental line CBI1582 and conventional non-transformed safflower. The compositional assessments determined the following concentrations:

A summary of the proximate chemical composition of seed from GOR73226, GOR73240, CBI1582 and several conventional safflower varieties are presented in Table 6.

### Moisture Content

Moisture content is an important factor associated with seed storage quality and was observed to be significantly different (p<0.05) across the varieties tested. The moisture content ranged from 4.6 to 6.8% (w/w). CBI1582 that had the highest average moisture content (6.2+0.27%) compared to the other varieties tested, but was not significantly different (p>0.01) to GOR73226 and GOR73240, or other high oleic acid varieties. The exception was that CBI1582 was significantly different (p<0.01) in moisture content to the linoleic safflower variety Sironaria.

GOR73226 (5.8+0.17%) and GOR73240 (5.6+0.10%) were not significantly different (p>0.01) to any of the safflower varieties tested and mean values were within the ranges reported in the literature.

The mean differences observed may be related to differences in maturity at harvest, but all varieties were within the industry standards for moisture content. It is recommended that for safe, long-term storage, threshed safflower seed should not exceed 8% moisture.

TABLE 5
Safflower seed composition analytes assessed
Proximates (6)

| | | |
|---|---|---|
| Crude Protein | Fat | Ash |
| Oven Moisture | Carbohydrates | Energy |

Vitamins (11)

| | | |
|---|---|---|
| Niacin B3 | Vitamin B6 | Vitamin C |
| Thiamine B1 | Riboflavin B2 | Panthothenate B5 |
| Vitamin A | Vitamin D2 | Vitamin B12 |
| Folate | Folic Acid | |

Minerals (9)

| | | |
|---|---|---|
| Copper | Magnesium | Potassium |
| Iron | Manganese | Phosphorus |
| Sodium | Zinc | Calcium |

Total Amino Acids (16)

| | | |
|---|---|---|
| Alanine | Histidine | Proline |
| Arginine | Isoleucine | Serine |
| Aspartic Acid + Asparagine | Leucine | Threonine |
| Lysine | Methionine | Tyrosine |
| Glutamic Acid + Glutamine | Phenylalanine | Valine |
| Glycine | | |

Fatty Acid Profile (20)

| | | |
|---|---|---|
| Myristic acid C14:0 | Palmitic acid C16:0 | Palmitoleic acid C16:1 |
| Heptadecanoic acid C17:0 | Heptadecenoic acid C17:1 | Stearic acid C18:0 |
| Oleic acid C18:1 | Linoleic acid C18:2 | Linolenic acid C18:3 |
| Arachidic acid C20:0 | Eicosenoic acid C20:1 | Behenic acid C22:0 |
| Erucic acid C22:1 | Lignoceric acid C24:0 | Tetracosenoic acid C24:1 |
| Polyunsaturated Fat | Monounsaturated Fat | Saturated Fat |
| Trans Fat | Oil Content | |

Sugars Profile (1)

Total Free Sugars

Anti-Nutrients (2)

| | |
|---|---|
| Tannins | Cyanide |

TABLE 6
Proximate analysis of safflower seed

| | | | | | Montola | | Tolerance | Literature |
|---|---|---|---|---|---|---|---|---|
| Characteristic* | GOR73226 | GOR73240 | CBI1582 | S-317 | 2003 | Sironaria | interval** | range |
| Moisture | 5.8 ± 0.17^{ab} | 5.6 ± 0.1^{ab} | 6.2 ± 0.27^{a} | 5.5 ± 0^{ab} | 5.85 ± 0.18^{ab} | 5.1 ± 0.18^{b} | 2.42-8.50 | 5.5-7.4 |
| Crude Protein | 20.1 ± 2.55^{a} | 21.8 ± 1.1^{a} | 21.5 ± 4.2^{a} | 16.2 ± 0.0^{a} | 19.5 ± 9.7^{a} | 18.5 ± 4.45^{a} | 0-39.1 | 14.7-37.2 |
| Total lipid | 34.6 ± 1.3^{ab} | 34.7 ± 1.4^{ab} | 31.2 ± 1.2^{ab} | 30.5 ± 1.2^{a} | 37.7 ± 1.5^{b} | 37.7 ± 1.52^{b} | 1.0-60.0 | 13.7-29.0 |
| Carbohydrates | 35.85 ± 3.6^{a} | 34.2 ± 2.8^{a} | 37.7 ± 4.7^{a} | 45.1 ± 1.6^{a} | 34.3 ± 8.8^{a} | 36.7 ± 5.0^{a} | 3.60-66.7 | 22.5-48.9 |
| Ash | 3.1 ± 0.3^{a} | 3.19 ± 0.3^{a} | 2.9 ± 0.2^{a} | 2.7 ± 0.3^{a} | 3.3 ± 0.28^{a} | 2.2 ± 0.24^{a} | 0.50-6.6 | 3.4-4.2 |
| Energy | 535.2 ± 5.1^{ab} | 536.3 ± 4.2^{ab} | 517.5 ± 4.2^{ab} | 519.8 ± 4.9^{ab} | 554.3 ± 6.2^{bc} | 559.9 ± 6.2^{bc} | 283-696 | 490-507 |
| (calories/100 g) | | | | | | | | |

*Calculated by differences on a dry weight basis (g/100 g) unless otherwise stated. Values are means ± standard error. Means with the same letter are not significantly different.
**Tolerance intervals were calculated to contain, with 95% confidence, 99% of the values in the population. Negative values were corrected zero.

### Analysis of Safflower Seed

Nutritional analysis was conducted on safflower seed to confirm that the composition of GOR73226 and GOR73240 remained within the normal levels for safflower when compared to the parental line CBI1582 and conventional non-transformed safflower. The compositional assessments determined the following concentrations:

A summary of the proximate chemical composition of seed from GOR73226, GOR73240, CBI1582 and several conventional safflower varieties are presented in Table 6.

### Moisture Content

Moisture content is an important factor associated with seed storage quality and was observed to be significantly different (p<0.05) across the varieties tested. The moisture content ranged from 4.6 to 6.8% (w/w). CBI1582 that had the highest average moisture content (6.2+0.27%) compared to the other varieties tested, but was not significantly different (p>0.01) to GOR73226 and GOR73240, or other high oleic acid varieties. The exception was that CBI1582 was significantly different (p<0.01) in moisture content to the linoleic safflower variety Sironaria.

GOR73226 (5.8+0.17%) and GOR73240 (5.6+0.10%) were not significantly different (p>0.01) to any of the safflower varieties tested and mean values were within the ranges reported in the literature.

The mean differences observed may be related to differences in maturity at harvest, but all varieties were within the industry standards for moisture content. It is recommended that for safe, long-term storage, threshed safflower seed should not exceed 8% moisture.

### Crude Protein

Protein content varied from 16-21% across all of the varieties tested. There was no significant differences (p>0.01) in crude protein content and all were within the literature range. The analysis demonstrates that crude protein levels in GOR73226 and GOR73240 were not significantly different to the conventional safflower lines tested.

### Crude Fat

Crude fat contents of GOR73226 and GOR73240 were not significantly different (p>0.01) to the control, CBI1582 or several of the commercial safflower varieties. The high oleic variety S-317 was significantly different (p>0.01) to Montola 2003 and Sironaria. This demonstrated that the crude fat levels in GOR73226 and GOR73240 were not significantly different to the conventional safflower lines tested.

### Ash

The analysis demonstrated that in relation to ash content, there were no significant differences (p>0.01) between GOR73226 and GOR73240 and the conventional safflower lines tested.

### Carbohydrates

The total carbohydrate level was calculated by difference using the fresh weight derived data and the following equation: % carbohydrates=100%—(% protein+% fat +% moisture+% ash). No significant differences in carbohydrate content (p>0.01) were observed between GOR73226 and GOR73240 and the conventional safflower lines tested.

### Energy

Calories were calculated using the following equation: Calories (Kcal/100 g)=(4×% protein)+(9×% fat)+(4×% carbohydrates). The energy potential of GOR73226 and GOR73240 were not significantly different (p>0.01) to the control,

CBI1582 or several of the commercial safflower varieties. Some differences were observed between conventional safflower lines tested. The analysis demonstrated that the caloric potential for GOR73226 and GOR73240 were similar to the conventional safflower lines tested.

### Summary of Proximate Analysis

Proximate analysis was undertaken on seed from GOR73226 and GOR73240 and compared to the parental control and conventional safflower. The analysis demonstrated that both GOR73226 and GOR73240 were not significantly different to their parental line CBI1582 and the conventional safflower lines tested.

### Mineral Composition of Safflower Seed

A number of minerals are essential plant nutrients. Some are required in larger amounts (macronutrients) and some only in trace amounts (micronutrients). Both macro- and micro-nutrients were analysed in seed samples from GOR73226 and GOR73240 and compared with CBI1582 and commercial safflower varieties. Across the 9 minerals assayed, GOR73226 and GOR73240 were not significantly different (p>0.01) to the parental control CBI1582 and were similar to the conventional safflower lines tested. Mineral contents for GOR73226 and GOR73240 were within the literature range for safflower.

### Vitamin Analysis of Safflower Seed

Vitamin analysis was undertaken from composite samples from the Kalkee trial site only. Levels of vitamins in GOR73226 and GOR73240 were comparable to the parental control CBI1582 and conventional safflower lines tested. However, levels of Vitamin B6 were considerably higher in all safflower samples from this study compared to those reported in the literature for other oilseeds. Further, compared to other safflower varieties, the level of Vitamin B5 was lower in GOR73226 compared to the conventional safflower, more similar to levels in observed in canola seed. Vitamin B6 functions as a cofactor of many enzymes. In particular, pyridoxal 5′-phosphate, which is the active form of Vitamin B6, has multiple roles as a versatile cofactor of enzymes that are mainly involved in the metabolism of amino acid compounds. The data indicate that safflower is a good source of Vitamin B6.

### Amino Acid Analysis of Safflower Seed

A total of 16 amino acids were examined. No significant differences (p>0.01) were observed between GOR73226 and GOR73240 with any of the conventional safflower lines tested. The analysis demonstrated that the amino acid composition of GOR73226 and GOR73240 were not significantly different to the parental control CBI1582 and conventional safflower.

Fatty Acid Profile from Safflower Seed

The GOR73226 and GOR73240 safflower have been genetically modified to accumulate super-high levels of oleic acid in the seed. The fatty acid composition of homozygous seed from field grown safflower were analysed for their fatty acid profiles (Table 7). The analysis indicated that GOR73226 and GOR73240 seed were comparable to the parental line CBI1582 and conventional safflower lines tested except for oleic acid, linoleic and palmitic acid levels (Table 7). In this analysis, GOR73226 and GOR73240 exhibited approximately 92% oleic acid, the levels of linoleic acid were 1.2% and 1.6% and palmitic acid levels were 2.6% and 2.5% respectively. The lower levels of linoleic and palmitic acid reflecting the homozygosity of the GOR73226 and GOR73240 seed samples.

Collectively, the fatty acid analysis of seed from GOR73226 and GOR73240 demonstrated the efficacy and specificity of the down regulation of the CtFAD2.2 and CtFATB genes.

Analysis of Free Sugars from Safflower Seed

There were no significant differences (p>0.01) in free sugar levels between GOR73226 and GOR73240 compared to the conventional safflower lines tested.

### Analysis of Anti-Nutrients in Safflower Seed

### Tannins

Tannins are polyphenols that can bind to and precipitate proteins (Butler and Rogler 1992: Chung et al., 1998). In livestock diets, tannins may diminish weight gains, apparent digestibility and feed utilisation efficiency. These anti-nutritional effects have generally been attributed to inhibition by tannins of digestion of dietary proteins. Other effects associated with dietary tannin are systemic, requiring absorption of inhibitory material from the digestive tract into the body (Chung et al., 1998).

The levels of tannins in the safflower tested ranged from 0.08-0.41%. GOR73226 and GOR73240 were not significantly different (p>0.01) to CBI1582 and the conventional lines tested. The levels in GOR73226 and GOR73240 were lower than in other oilseeds e.g, canola-1.5%, Canadian Canola Council (2015): rapeseed-0.5%, Heuzé et al. (2017a): soybean-0.85%, Heuzé et al. (2017b): sunflower meal-1.4%, Heuzé et al. (2016).

### Hydrogen Cyanide

Prussic acid, also known as hydrogen cyanide or HCN, is a chemical compound both useful and dangerous. Although it is naturally present in some plants, this substance can also be synthesised through a variety of chemical processes. It has been reported that 110 to 135 ppm may be fatal after 0.5 to 1 hour or later, or dangerous to life. The acute lethal dosage of hydrogen cyanide (HCN) in most animal species is ˜2 mg/kg with plant materials containing ≥200 ppm of cyanogenic glycosides considered dangerous (Cope, 2017). On a dry weight basis, plant materials with 200-500 ppm HCN should be considered potentially toxic to livestock (Williams, 2012).

All safflower varieties examined contained less than 2.5 ppm (mg/Kg). The analysis demonstrated that GOR73226 and GOR73240 were not significantly different to the conventional safflower lines tested and that potential cyanide production among varieties is very low and not considered toxic to either humans or animals.

TABLE 7
Fatty acid profile of GOR73226 and GOR73240
compared to the parental control CBI1582

| | | | | S- | | |
|---|---|---|---|---|---|---|
| Fatty acid* | GOR73226 | GOR73240 | CBI1582 | 317 | Montola | Sironaria |
| Myristic acid | 0.03 | 0.03 | 0.09 | 0.07 | 0.08 | 0.11 |
| C14:0 | | | | | | |
| Palmitic acid | 2.6 | 2.5 | 5.8 | 5.5 | 5.3 | 7.4 |
| C16:0 | | | | | | |
| Palmitoleic acid | 0.2 | 0.1 | 0.2 | 0.2 | 0.1 | 0.1 |
| C16:1 | | | | | | |
| Heptadecanoic | <0.1 | <0.1 | <0.1 | <0.1 | <0.1 | <0.1 |
| acid C17:0 | | | | | | |
| Heptadecenoic | <0.1 | <0.1 | 0.1 | 0.1 | <0.1 | <0.1 |
| acid C17:1 | | | | | | |
| Stearic acid | 2.5 | 2.0 | 2.2 | 2.9 | 2.0 | 2.4 |
| C18:0 | | | | | | |
| Oleic acid C18:1 | 92.0 | 92.3 | 75.2 | 67.8 | 74.8 | 11.8 |
| Linoleic acid | 1.2 | 1.6 | 14.7 | 21.9 | 16.1 | 76.3 |
| C18:2 | | | | | | |
| Linolenic acid | 0.1 | 0.1 | 0.1 | 0.1 | 0.1 | 0.1 |
| C18:3 | | | | | | |
| Arachidic acid | 0.5 | 0.4 | 0.5 | 0.5 | 0.4 | 0.3 |
| C20:0 | | | | | | |
| Eicosenoic acid | 0.3 | 0.3 | 0.3 | 0.3 | 0.3 | 0.2 |
| C20:1 | | | | | | |
| Behenic acid | 0.3 | 0.3 | 0.4 | 0.3 | 0.3 | 0.2 |
| C22:0 | | | | | | |
| Erucic acid | <0.1 | <0.1 | <0.1 | <0.1 | <0.1 | <0.1 |
| C22:1 | | | | | | |
| Lignoceric acid | 0.2 | 0.2 | 0.2 | 0.2 | 0.3 | 0.5 |
| C24:0 | | | | | | |
| Tetracosenoic | 0.1 | 0.2 | 0.2 | 0.1 | 0.2 | 0.6 |
| acid C24:1 | | | | | | |
| Polyunsaturated | 1.3 | 1.7 | 14.8 | 22.0 | 16.2 | 76.4 |
| fatty acids | | | | | | |
| Monounsaturated | 92.6 | 92.9 | 76.0 | 68.5 | 75.4 | 12.7 |
| fatty acids | | | | | | |
| Saturated fatty | 6.1 | 5.4 | 9.2 | 9.5 | 8.4 | 10.9 |
| acids | | | | | | |

*Seed samples were analysed by NSW Department of Primary Industries Oil testing Services and CSIRO. Fatty acid levels are presented as % of total fatty acid content, by weight. Homozygous seed samples for GOR73226 and GOR73240 were obtained from field trials conducted at Kununurra, Western Australia, in 2016.

### Analysis of Safflower Meal

### Feed Quality Analysis

A feed quality assessment of safflower meal from GOR73226 and GOR73240, the CBI1582 parental control and conventional safflower was undertaken. GOR73226 and GOR73240 were not significantly different to the parental control CBI1582 and the conventional safflower lines tested.

### Analysis of Vegetative Tissue

Safflower is valuable forage for Mediterranean areas since it remains green and has a higher feed value under dry conditions (Stanford et al., 2001; Landau et al., 2005; Peiretti 2009). The potential value to Australian farming systems is poorly understood, however several reports indicate strategic use can offer satisfactory growth rates and productivity to livestock (French et al., 1988). Safflower can be directly grazed by sheep and cattle or fed fresh in a cut-and-carry system. Safflower is also used as hay especially if it has suffered from frost. It has been recommended that silage should be prepared from safflower at the budding stage (Peiretti, 2009; Oyen et al., 2007).

The GOR73226 and GOR73240 plants may present a forage opportunity, particularly during drought where seed remaining in the safflower stubble following harvest germinates on an early rainfall event, generating early vegetative growth available for livestock grazing in autumn. Therefore, the forage quality of GOR73226 and GOR73240 was examined.

### Feed Quality Analysis

The feed quality analysis indicated that vegetative tissues from GOR73226 and GOR73240 were not significantly different to the conventional safflower. The majority of the components examined were within the literature ranges reported for vegetative safflower tissue. Of note, several components differed to those reported elsewhere. For example, the protein levels of GOR73226 and GOR73240 were much higher than reported in the literature. However, it is noted that protein levels are dependent on growth stage and agronomic conditions, with higher crude protein levels reportedly associated with nitrogen application (Danieli et al., 2011) and decreases in crude protein recorded through later stages of development (Corleto et al., 2005; Peiretti, 2009).

### Mineral Content

Mineral nutrition is a key component in maintaining the health and productivity of livestock. Therefore, understanding and balancing the mineral nutrient composition of a feed source can be important. Mineral deficiencies are more likely to occur than toxicities and feed rations are often formulated to exceed minimum animal requirements. In these cases, it is important to determine if dietary mineral concentrations are beyond maximum tolerable concentrations for animals. Mineral toxicities resulting from an over-supply in feed or water may have observable effects such as a decrease in animal performance or a change in animal behaviour.

The levels of key minerals in the vegetative tissue of GOR73226 and GOR73240 were examined. None of the minerals assayed were close to or over the maximum tolerable levels for livestock.

### Conclusions

A comprehensive analysis of the composition of GOR73226 and GOR73240 was conducted. Results were compared with the parental control CBI1582 and commercially available safflower varieties. The compositional and feed quality analyses confirmed that GOR73226 and GOR73240 were not significantly different to other safflower varieties, except having a fatty acid profile with high levels of oleic acid and low levels of linoleic acid and palmitic acid, the intended trait.

This compositional analysis and feed quality analysis confirmed that GOR73226 and GOR73240 were as safe for human and animal consumption respectively when compared to conventional safflower lines tested and/or reference literature.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abdullah et al. (1986) Biotech. 4:1087.
Allen et al. (2007) PNAS 104:16371-16376.
Ascherio and Willett (1997) Am. J. Clin. Nutr. 66:1006S-1010S.
Axtell (2013) RNA 19:740-751.
Bartholomew (1971) Temperature effects on the fatty acid composition of developing seeds in safflower, Carthamus tinctorius L. Thesis, University of California, 1971.
Belide et al. (2011). Plant Methods 7:12.
Bligh and Dyer (1959). Canadian Journal of Biochemistry and Physiology 37:911-7.
Bowers et al. (2016) G3 Genes|Genomes|Genetics 6:2203-2211.
Bonanome and Grundy (1988). N. Engl. Med. 318: 1244-1248.
Broun et al. (1999) Annu. Rev. Nutr. 19: 197-216.
Butler and Rogler (1992) Biochemical Mechanisms of the Antinutritional Effects of
Tannins. In: Phenolic Compounds in Food and Their Effects on Health I, Chapter 23, pp 298-304.
Canadian Canola Council (2015). Canola Meal Feed Industry Guide, http://www.canolacouncil.org/media/516716/2015_canola_meal_feed_industry_guide, pdf.
Cao et al. (2013) BMC Plant Biol. 13:5.
Chung et al. (1998) Crit Rev Food Sci Nutr 38:421-464.
Corleto et al. (2005) Evolution of biomass and quality of safflower during the reproductive stage for hay and ensiling purposes. In: Proceedings of the 6th International Safflower Conference, Istanbul, Turkey, pp. 69-73.
Coutu et al. (2007) Transgenic Research 16:771-781.
Cope et al. (2017) Overview of Cyanide Poisoning. Merck Veterinary Manual (http://www.merckvetmanual.com/toxicology/cyanide-poisoning/overview-of-cyanide-poisoning).
Danieli et al. (2011) Italian J Agron 6:e4.
Dougherty et al. (1995). Am. J. Clin. Nutr. 61:1120-1128.
Fenandez San Juan (1995). Alimentaria 33: 93-98.
French et al. (1988) Tropical Grasslands 22:85-90.
Fujimura et al. (1985) Plant Tissue Culture Lett. 2:74.
Gamez et al. (2003) Food Res International 36: 721-727.
Gunstone (2001) Inform 11: 1287-1289.
Helliwell and Waterhouse (2005) Methods Enzymol 392:24-35.
Heuze et al. (2016) Sunflower meal. Feedipedia, a programme by INRA, CIRAD, AFZ and FAO, http://www.feedipedia.org/node/732.
Heuze et al. (2017a) Rapeseed meal. Feedipedia, a programme by INRA, CIRAD, AFZ and FAO, http://www.feedipedia.org/node/52.
Heuze et al. (2017b) Soybean seeds. Feedipedia, a programme by INRA, CIRAD, AFZ and FAO, http://www.feedipedia.org/node/42.
Hill and Knowles (1968). Crop Sci. 8:275-277.
Hu et al. (1997) N. Engl. J. Med. 337: 1491-1499.
Knowles (1968) Economic Botany 22: 195-200.
Knowles (1972) Journal of the American Oil Chemists' Society 49:27-29.
Knowles (1989) Oil crops of the world: their breeding and utilization/ed G. Robbelen,
R. Keith Downey, A.Ashri. McGraw-Hill, New York.
Landau et al. (2005 Small Ruminant Res 59:239-249.)
Langridge et al. (2001) Aust J Agric Res 52: 1043-1077.
Lemieux (2000) Current Genomics 1: 301-311.
Liu et al. (2002). J. Am. Coll. Nutr. 21: 205S-211S.
Marangoni et al. (1995) Trends in Food Sci. Technol. 6: 329-335.
Mensink and Katan (1990) N. Engl. J. Med. 323: 439-445.
Noakes and Clifton (1998) Am. J. Clin. Nutr. 98: 242-247.
Oelke et al. (1992) Safflower. Alternative Field Crop Manual, University of Wisconsin-Exension, Cooperative Extension.
Oil World Annual (2004) International Seed Testing Association (ISTA) Mielke GmbH, Hamburg, Germany.
Oyen and Umali (2007) Carthamus tinctorius L., Record from Protabase, van der
Vossen, H. A. M.: Mkamilo, G. S. (Editors). PROTA (Plant Resources of Tropical Africa/Ressources végétales de l′Afrique tropicale), Wageningen, Netherlands. Peiretti (2009) Livestock Research for Rural Development. Volume 22, Article #206. Perez-Vich et al. (1998) JAOCS 75:547-555.
Phillips et al. (2002) Journal of Food Composition and Analysis 12:123-142.
Reynolds et al. (2015) Front Plant Sci 6, Artcile number 164.
Roche and Gibney (2000) Am. J. Clin. Nutr. 71: 232S-237S.
Ryan et al. (1984) J. Am. Oil Chem. Soc. 61:1610-1619.
Scarth and Tang (2006) Crop Science 46:1225-1236.
Sebedio et al. (1994) Fett. Wiss. Technol. 96: 235-239.
Smith et al. (2000) Nature 407: 319-320.
Stanford et al. (2001) Can J Anim Sci 81:289-292.
Thelen and Ohlrogge (2002) Metabolic Engineering 4: 12-21.
Tholstrup et al. (1994) Am. J. Clin. Nutr. 59: 371-377.
Toriyama et al. (1986) Theor. Appl. Genet. 205:34.
Waterhouse et al. (1998) Proc. Natl. Acad. Sci. USA 95: 13959-13964.
Williams (2012) Medicinal Plants in Australia Volume 3: Plants, Portions and Poisons. Australia: Rosenberg Publishing.
Zhou et al. (2011) Journal of Biological Chemistry 286:2877-2885.
Zhou et al. (2013) FEBS Lett 587:2371-2376.
Zock and Katan (1992) J. Lipid Res. 33:399-410.

1. A safflower plant cell comprising
(a) a polynucleotide which comprises a sequence of nucleotides provided as SEQ ID NO: 13 and a polynucleotide which comprises a sequence of nucleotides provided as SEQ ID NO: 14,
(b) a polynucleotide which comprises a sequence of nucleotides provided as SEQ ID NO: 21 and a polynucleotide which comprises a sequence of nucleotides provided as SEQ ID NO: 22,
(c) a T-DNA molecule, wherein the T-DNA molecule is inserted into a region of a chromosome of the cell, wherein the region has a nucleotide sequence provided as SEQ ID NO: 11,
(d) a T-DNA molecule, wherein the T-DNA molecule is inserted into a region of a chromosome of the cell, wherein the region has a nucleotide sequence provided as SEQ ID NO: 18, or
(e) any combination thereof.

2. The safflower plant cell of claim 1 which comprises (a) and (c), or (b) and (d), and/or which is homozygous for the polynucleotide (s) and/or the T-DNA (s).

3. A safflower plant cell comprising comprising one or more of
(a) a polynucleotide which comprises a sequence of nucleotides provided as SEQ ID NO: 7,
(b) a polynucleotide which comprises a sequence of nucleotides provided as nucleotides 296 to 8640 of SEQ ID NO: 7,
(c) a polynucleotide which comprises a sequence of nucleotides provided as SEQ ID NO: 33,
(d) a polynucleotide which comprises a sequence of nucleotides provided as SEQ ID NO: 34, or
(e) a polynucleotide having at least 95% identity with the full length of nucleotides 296 to 8640 of SEQ ID NO: 7.

4. (canceled)

5. The safflower plant cell according to claim 1, wherein one or more or all of the following apply
(a) oleic acid comprises at least 90% by weight of the total fatty acids in the cell, preferably between 908 and 958 by weight of the total fatty acids in the cell,
(b) at least 95% by weight of the lipid in the cell is triacylglycerol (TAG),
(c) palmitic acid comprises less than 2.78 by weight of the total fatty acids in the cell, preferably between 2.5% and 2.7% by weight of the total fatty acids in the cell,
(d) linoleic acid comprises less than 1.78 by weight of the total fatty acids in the cell, preferably between 1.2% and 1.6% by weight of the total fatty acids in the cell,
(e) α-linolenic acid is absent from the total fatty acids in the cell or is present at less than 0.28 by weight of the total fatty acids in the cell,
(f) the cell comprises a hygromycin phosphotransferase polypeptide,
(g) the cell has reduced CtFAD2-2 activity and reduced CtFATB-3 activity relative to a corresponding safflower cell lacking the polynucleotide (s) and/or the T-DNA (s),
(h) the cell comprises an ol allele of the CtFAD2-1 gene or an oll allele of the CtFAD2-1 gene, or both alleles, preferably is homozygous for the ol allele.

6. The safflower plant cell according to claim 1 which is a seed cell, preferably in a seed of a safflower plant growing in a field or in a harvested seed.

7. Safflower seed comprising a cell according to claim 1, or a collection of safflower seeds, wherein at least 95% of the seeds are seeds having the cell, or seedmeal extracted from the safflower seed.

8. A safflower plant, or part thereof, which comprises a cell according to claim 1 and/or which produces seed having the cell.

9. The safflower plant, or part thereof, of claim 8, produced by growing the seed having the cell.

10. The safflower plant, or part thereof, of claim 8, wherein the polynucleotide (s) and/or the T-DNA (s) are stably integrated into the genome of the plant, or part thereof, for at least nine generations, or
wherein one or more or all of the following features are the same as a corresponding plant lacking the polynucleotide (s) and/or the T-DNA (s) grown under the same conditions: seedling vigour, plant height, time to flowering, harvest lodging, seed crude protein content, seed crude fat content, seed ash content and seed carbohydrate content, or
which comprises a transgene other than the polynucleotide (s) and/or the T-DNA (s).

11-12. (canceled)

13. Pollen or an ovule of the plant according to claim 8.

14. A tissue culture of regenerable cells, wherein the cells are as defined in claim 1, preferably being of a tissue selected from the group consisting of leaves, pollen, embryos, roots, root tips, pods, flowers, ovules and stems.

15. A method for producing a safflower plant or seed therefrom, the method comprising:
(a) crossing a first safflower plant according to claim 8 with a second safflower plant to yield progeny safflower seed; and
(b) growing the progeny safflower seed, under plant growth conditions, to yield a progeny safflower plant, and optionally
(c) harvesting seed from the progeny safflower plant.

16. The method of claim 15, wherein the second safflower plant has at least one agronomically desirable trait that is lacking in the first safflower plant, or
wherein the trait is herbicide resistance, insect resistance, bacterial disease resistance, fungal disease resistance, viral disease resistance, female sterility or male sterility, or optionally further comprising:
(d) backcrossing one or more progeny plants from step (b) with plants of the same genotype as the second safflower plant for a sufficient number of times to produce a plant having at least 75% of the genotype of the second safflower plant.

17-18. (canceled)

19. A method of identifying a safflower plant,
the method comprising analysing DNA obtained from the plant for one or more of the polynucleotide (s) and/or T-DNA molecules defined in claim 1.

20. A method of producing safflower seed, the method comprising,
a) growing a plant according to claim 8, preferably in a field as part of a population of at least 1000 such plants, and
b) harvesting the seed.

21. A method of producing safflower oil, comprising obtaining seed of claim 7 and processing the seed to obtain safflower oil.

22. Oil obtained from, or obtainable by, one or more of the cell according to claim 1, the seed of claim 7, the safflower plant according to claim 8, or the method of claim 21.

23. A composition, preferably a food or feed composition, comprising one or more of the cell according to claim 1, the seed of claim 7, the safflower plant according to claim 8, the oil of claim 22, or oil produced using the method of claim 21, and one or more acceptable carriers.

24. (canceled)

25. A method of producing a feedstuff, the method comprising admixing one or more of the cell according to claim 1, the seed of claim 7, the safflower plant according to claim 8, the oil of claim 22, the composition of claim 23 or oil produced using the method of claim 21, with at least one other food ingredient, or heating a food product in the presence of the oil of claim 22, the composition of claim 23 or oil produced using the method of claim 21.

26. (canceled)

27. Feedstuffs, cosmetics or chemicals comprising reacting one or more of the cell according to any one of claims 1 to 6, the seed of claim 7, the safflower plant according to any one of claims 8 to 11, the oil of claim 22, the composition of claim 23 or oil produced using the method of claim 21.

28. (canceled)


SEQUENCE LISTING (40 sequences)

SEQ ID NO: 1 - protein, 383 aa, Carthamus tinctorius
MGAGGRMSNPSEGEKKTELEGIQRVPYQKPPFTVGDVKKAIPPHCFNRSVIRSFSYVVYDLTIASILYYIATTFIPLLPHPLAYVAWPIYWAVQGCVMTGVWVIAHECGHHAFSDYQWLDDTVGLILHSVLLVPYFSWKYSHRRHHSNTGSIEHDEVFVPKLKSGVRSTAKYLNNPPGRILTLLVTLTLGWPLYLMFNVSGRYYDRFACHFDPNSPIYSNRERAQIFISDAGIFAVLYGLYRLAAVKGLVWVLTVYAGPLLVVNGFLVLITFLQHTHPSLPHYDSTEWDWLRGALATIDRDYGILNKVFHNITDTHVTHHLFSTMPHYHAMEATKAIIPILGDYYQFDGTSVFKAMYRETKECIYVDKDEEVKDGVYWYRNKI

SEQ ID NO: 2 - DNA, 1456 bp, Carthamus tinctorius
gcacgaggcccaaacggcccatttcgctattcaaaaggagtttcagaaagcctccaaagattcattcaggtcattgaacaatgggtgcaggcgggcgaatgtcgaacccctccgagggcgaaaagaaaaccgaactcgaaggcatccaacgagtcccataccaaaaacctccgttcacagtcggggatgtcaaaaaagccatcccacctcattgtttcaatcgatcggtcatccgctctttctcgtacgtcgtttacgaccttaccatcgcatccatcttgtactacatcgccacaactttcatcccgctcctcccgcacccgctcgcctacgtggcctggcccatttactgggccgttcagggctgtgtcatgaccggggtctgggtcatagcccacgaatgcggccatcacgcctttagcgactaccaatggctcgacgacactgtcggccttatcctacattctgttcttcttgttccttacttctcgtggaaatacagccaccgccgccaccactccaacaccggctcgattgagcacgacgaggtcttcgtcccgaaactcaaatccggggtccggtccaccgcaaaatacctaaacaacccccccggccggatcttgacccttcttgtcaccctcaccctcggctggccgttgtacctcatgttcaacgtctccggccgctactacgaccgctttgcctgccattttgacccaaacagccccatctactcgaaccgtgaacgtgcccagatcttcatctccgacgccgggatcttcgccgtcctctacgggctctaccggctcgcggcggtcaaagggctcgtgtgggtcctgaccgtctatgccggaccgttgctcgtggtcaacgggtttcttgtcctgatcacgttcctccagcacacccacccgtcgttgccgcactacgactcaaccgagtgggactggctccgtggggccctggccaccatcgaccgtgactacgggattttaaacaaggtgttccacaacatcaccgacacccacgtgacccaccatctgttttcgacgatgccgcattatcacgcgatggaggccacgaaggcgataattccgatcctcggggattattatcagttcgatgggacgtcagtttttaaggcgatgtatagggagacgaaggagtgcatttatgtggataaagatgaggaggtgaaggatggggtttactggtaccggaataagatttgaagatgatggatgaaatgaaaaaatggtattaatagagtggcgaaatggtgtctttaattagtgttgttttagagtatggttttatgagttatgatatgccatttaaatggacatggaattgttagtgatttgaataacttttgtactctgaatatggtgtttgtaggctatgtagccactctgtttttagctttttaaatgcatctatctatgttggaacctta

SEQ ID NO: 3 - protein, 361 aa, Carthamus tinctorius
MVATAATASLFPVSSPQPHSGTKKSGKLGGVPDGVDVRGIKTKSVTSGGLQVKAAQAPAEVNGTRVRPMIKTDDNSTSHVPRTFINQLPDWSMLLAAITTIFLAAEKQWMMLDWKTKRPDMLADLDPFGLGRIVQDGLVFRQNFSIRSYEIGADRTASIETLMNHLQETALNHVKTAGLLGDGFGSTPEMCKKNLFWVVTKMQVMVDRYPTWGDVVQVDTWVARSGKNGMRRDWLLRDCKTGEVVTRASSNWVMMNKETRRLSKIPDEVRAEIEQYFVDAPPVVDDDSRKLPKLEDTTADYVRNGLTPRWSDLDVNQHVNNVKYIGWILESAPQQVVEKYELAGLTLEYRRECRKDSVLSR

SEQ ID NO: 4 - DNA, 1671 bp, Carthamus tinctorius
gcacgaggctggccctcctccttctctctctctcttctttcagcttccctgcctgctaaaattccactaaacaaatacaaaaggaatccaaattcaacattcttccccaatcgcacgccttagggatatatgctgttctttggtcatctttgaacaattatttataatttggtgagcacgtctgaaaccaataggtttgactgataaacatacgggttaatctgaatactttaatcatggttgctacggctgctactgcatcactgtttccggtttcttctccacaacctcactctgggaccaagaaatctggtaagcttggaggtgtgccagatggcgttgatgtgcgtggaatcaagacaaaatctgttacttctggcggtttgcaagttaaggctgcacaggctcctgcagaggtaaatgggactagagtgcggcccatgatcaagacagacgataattctacgtcacatgtcccaagaacctttatcaaccagttgccggattggagcatgcttcttgctgcaatcaccacaatcttcttggctgcagagaaacaatggatgatgctggattggaaaaccaagcggcctgacatgcttgctgatctggatccttttggtttagggagaattgtgcaggatgggcttgtattccgtcaaaacttctctattagatcctatgaaataggggctgatcgaactgcgtcgatagagacgctaatgaatcatttgcaggaaactgcccttaatcatgtaaagactgcgggactcttgggtgatggctttggctcaacacctgaaatgtgcaagaagaatctattctgggtggtgacaaagatgcaggtgatggtagaccgttatccaacttggggtgatgttgttcaagtagatacatgggtagctcgatctgggaaaaacggcatgcgccgtgattggctgcttcgtgattgcaaaacaggcgaggtcgtaacaagagcttcaagtaattgggttatgatgaataaagagacaaggaggttatcaaaaattccagatgaagtccgagctgaaatagaacagtactttgtagatgcacctccggtcgtggatgatgacagcagaaaattgcctaaacttgaagacaccactgctgactatgttcgcaatggtttaactccaaggtggagtgatctggatgtcaaccaacatgttaacaatgtgaagtatattggttggatccttgagagtgctccacaacaagttgtggagaagtatgagcttgctggcttgactctggaataccgtcgagaatgtaggaaggatagcgtgctgagtcgctgacatcggtattgggtggcaatggtgatggaatagctgattccgattccagtgatgttgattgccagcatgtgctgctatttgagagtggtggtgaaggtgaggttgtgaaaggaaggactgcatggcgaccaaaaaaagataaggatggaattagaagcagcattgctgacttctttgctggaaatggaaatggaaatggaagtgcctaataccatgtttcatgttttgcccttttgcctcatttcaatgtgtataaaagaatcatcatcactcatcctatttatgctggctcttggaaataacttgcaatgtcctgcccccacatatatacatatttttctattttgta

SEQ ID NO: 5 - DNA, 756 bp, Artificial Sequence
  Nucleotide sequence of the region of the cDNA for the safflower FAD2-2 gene used in the genetic construct pCW732
taccaatggctcgacgacactgtcggccttatcctacattctgttcttcttgttccttacttctcgtggaaatacagccaccgccgccaccactccaacaccggctcgattgagcacgacgaggtcttcgtcccgaaactcaaatccggggtccggtccaccgcaaaatacctaaacaacccccccggccggatcttgacccttcttgtcaccctcaccctcggctggccgttgtacctcatgttcaacgtctccggccgctactacgaccgctttgcctgccattttgacccaaacagccccatctactcgaaccgtgaacgtgcccagatcttcatctccgacgccgggatcttcgccgtcctctacgggctctaccggctcgcggcggtcaaagggctcgtgtgggtcctgaccgtctatgccggaccgttgctcgtggtcaacgggtttcttgtcctgatcacgttcctccagcacacccacccgtcgttgccgcactacgactcaaccgagtgggactggctccgtggggccctggccaccatcgaccgtgactacgggattttaaacaaggtgttccacaacatcaccgacacccacgtgacccaccatctgttttcgacgatgccgcattatcacgcgatggaggccacgaaggcgataattccgatcctcggggattattatcagttcgatgggacgtcagtttttaaggcgatgtatagggagacgaaggagtgcatttatgtggat

SEQ ID NO: 6 - DNA, 412 bp, Artificial Sequence
  Nucleotide sequence of the region of the cDNA for the safflower FATB-3 gene used in the genetic construct pCW732
cttctggcggtttgcaagttaaggctgcacaggctcctgcagaggtaaatgggactagagtgcggcccatgatcaagacagacgataattctacgtcacatgtcccaagaacctttatcaaccagttgccggattggagcatgcttcttgctgcaatcaccacaatcttcttggctgcagagaaacaatggatgatgctggattggaaaaccaagcggcctgacatgcttgctgatctggatccttttggtttagggagaattgtgcaggatgggcttgtattccgtcaaaacttctctattagatcctatgaaataggggctgatcgaactgcgtcgatagagacgctaatgaatcatttgcaggaaactgcccttaatcatgtaaagactgcgggactcttgggtgatgg

SEQ ID NO: 7 - DNA, 9266 bp, Artificial sequence
  Nucleotide sequence of the region of the genetic construct pCW732 spanning the T-DNA
ggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgctagccaattggggcccgcgttgggaattcgattgagctctaatacgactcactataggcggccgctctagactcaagcatacggacaagggtaaataacatagtcaccagaacataataaacaaaaagtgcagaagcaagactaaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaattctcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcagacccatctgtgccactcgtacaattacgagagttgttttttttgtgattttcctagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccaccatggatccctgcagtaaatcccgggctcgagacaagtttgtacaaaaaagcaggctccgcggccgcccatcacccaagagtcccgcagtctttacatgattaagggcagtttcctgcaaatgattcattagcgtctctatcgacgcagttcgatcagcccctatttcataggatctaatagagaagttttgacggaatacaagcccatcctgcacaattctccctaaaccaaaaggatccagatcagcaagcatgtcaggccgcttggttttccaatccagcatcatccattgtttctctgcagccaagaagattgtggtgattgcagcaagaagcatgctccaatccggcaactggttgataaaggttcttgggacatgtgacgtagaattatcgtctgtcttgatcatgggccgcactctagtcccatttacctctgcaggagcctgtgcagccttaacttgcaaaccgccagaagcctgcaggtaccaatggctcgacgacactgtcggccttatcctacattctgttcttcttgttccttacttctcgtggaaatacagccaccgccgccaccactccaacaccggctcgattgagcacgacgaggtcttcgtcccgaaactcaaatccggggtccggtccaccgcaaaatacctaaacaacccccccggccggatcttgacccttcttgtcaccctcaccctcggctggccgttgtacctcatgttcaacgtctccggccgctactacgaccgctttgcctgccattttgacccaaacagccccatctactcgaaccgtgaacgtgcccagatcttcatctccgacgccgggatcttcgccgtcctctacgggctctaccggctcgcggcggtcaaagggctcgtgtgggtcctgaccgtctatgccggaccgttgctcgtggtcaacgggtttcttgtcctgatcacgttcctccagcacacccacccgtcgttgccgcactacgactcaaccgagtgggactggctccgtggggccctggccaccatcgaccgtgactacgggattttaaacaaggtgttccacaacatcaccgacacccacgtgacccaccatctgttttcgacgatgccgcattatcacgcgatggaggccacgaaggcgataattccgatcctcggggattattatcagttcgatgggacgtcagtttttaaggcgatgtatagggagacgaaggagtgcatttatgtggataagggtgggcgcgccgacccagctttcttgtacaaagtggtctcgatcgaggaattcggtaccccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattatttttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaagctgggttcgaaatcgataagcttgcgctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcacaagcttggatcctctagaccactttgtacaagaaagctgggtcggcgcgcccacccttatccacataaatgcactccttcgtctccctatacatcgccttaaaaactgacgtcccatcgaactgataataatccccgaggatcggaattatcgccttcgtggcctccatcgcgtgataatgcggcatcgtcgaaaacagatggtgggtcacgtgggtgtcggtgatgttgtggaacaccttgtttaaaatcccgtagtcacggtcgatggtggccagggccccacggagccagtcccactcggttgagtcgtagtgcggcaacgacgggtgggtgtgctggaggaacgtgatcaggacaagaaacccgttgaccacgagcaacggtccggcatagacggtcaggacccacacgagccctttgaccgccgcgagccggtagagcccgtagaggacggcgaagatcccggcgtcggagatgaagatctgggcacgttcacggttcgagtagatggggctgtttgggtcaaaatggcaggcaaagcggtcgtagtagcggccggagacgttgaacatgaggtacaacggccagccgagggtgagggtgacaagaagggtcaagatccggccgggggggttgtttaggtattttgcggtggaccggaccccggatttgagtttcgggacgaagacctcgtcgtgctcaatcgagccggtgttggagtggtggcggcggtggctgtatttccacgagaagtaaggaacaagaagaacagaatgtaggataaggccgacagtgtcgtcgagccattggtacctgcaggcttctggcggtttgcaagttaaggctgcacaggctcctgcagaggtaaatgggactagagtgcggcccatgatcaagacagacgataattctacgtcacatgtcccaagaacctttatcaaccagttgccggattggagcatgcttcttgctgcaatcaccacaatcttcttggctgcagagaaacaatggatgatgctggattggaaaaccaagcggcctgacatgcttgctgatctggatccttttggtttagggagaattgtgcaggatgggcttgtattccgtcaaaacttctctattagatcctatgaaataggggctgatcgaactgcgtcgatagagacgctaatgaatcatttgcaggaaactgcccttaatcatgtaaagactgcgggactcttgggtgatgggcggccgcggagcctgcttttttgtacaaacttgtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagggcccgacgtcgcatgctcctctagactcgacgcgtcctagagatccgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgaccccgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccggagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcatcgatgatatcagatccgggcggaaataggtaaagaagttgcggataaggtaattgccattgcagattatttggattgagagtgaatatgagactctaattggataccgaggggaatttatggaacgtcagtggagcatttttgacaagaaatatttgctagtgatgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccacaaaatcaccactcgatacaggcagcccatcagtccactagagcgatcgcagctttctgagccgccgattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcgatttgaccgatcttctccagtttttctcggttgctgaggccgc

SEQ ID NO: 8 - protein, 341 aa, Proteus mirabilis
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 9 - DNA, 41 bp, Artificial Sequence
  Nucleotide sequence of the Right border sequence inserted into GOR73226 (pCW732_RB)
aaacactgatagtttaaactgaaggcgggaaacgacaatct

SEQ ID NO: 10 - DNA, 122 bp, Artificial Sequence
  Nucleotide sequence of the GOR73226 Right border junction sequence (E26_RB_junction)
atacatatatatagggttaggttattttgttaacccattaatttgtgttaaccgtgttaaccatataaccaccgttattttaaacactgatagtttaaactgaaggcgggaaacgacaatct

SEQ ID NO: 11 - DNA, 272 bp, Artificial Sequence
  Nucleotide sequence of a Safflower scaffold sequence into which the T-DNA of GOR73226 has inserted (gx_S317_Scaff_m9987)
atacatatatatagggttaggttattttgttaacccattaatttgtgttaaccgtgttaaccatataaccaccgttgatcatttcaatcaatggttaagattaatttcctaaattagaggaaagttatttaaatcctaaaatataggaggctggcccacatatgcacaaaaataggaaaaataaagaaatcctattggtccatttttggttaacacggttaacacaaattcatgggttaacaaaataactctcctctatatatatatatata

SEQ ID NO: 12 - DNA, 145 bp, Artificial Sequence
  Nucleotide sequence of the GOR73226 Left border junction sequence (E26_LB_junction)
acaggttgaaccgcagctctcttccattgataataggtctgaacggaaatacccacgatcttaacggcgtccttcaaggttgcgccgccagcgacctgagcttcatgggttaacaaaataactctcctctatatatatatatata

SEQ ID NO: 13 - DNA, 104 bp, Artificial Sequence
  Nucleotide sequence of the Left border sequence inserted into GOR73226 (E26_LB)
acaggttgaaccgcagctctcttccattgataataggtctgaacggaaatacccacgatcttaacggcgtccttcaaggttgcgccgccagcgacctgagcttc

SEQ ID NO: 14 - DNA, 40 bp, Artificial Sequence
  Nucleotide sequence of a portion of the GOR73226 Right border junction
catataaccaccgttattttaaacactgatagtttaaact

SEQ ID NO: 15 - DNA, 40 bp, Artificial Sequence
  Nucleotide sequence of a portion of the GOR73226 Left border junction
ccgccagcgacctgagcttcatgggttaacaaaataactc

SEQ ID NO: 16 - DNA, 39 bp, Artificial Sequence
  Nucleotide sequence of the Right border sequence (pCW732_RB) inserted into GOR73240
acactgatagtttaaactgaaggcgggaaacgacaatct

SEQ ID NO: 17 - DNA, 209 bp, Artificial Sequence
  Nucleotide sequence of the GOR73240 Right border junction sequence (E40_RB_junction)
taagaaatggcggaacaaaattacaccgttattccggcgtccgagcatagaagaaaagcggatatttggcaatgttacgacttgtgcaagtttcccgatggtagcaaaaaagtgcgatgcaatcggtgtggcgactttctaaaacccgaatcgaattcgacgttaaagcgacactgatagtttaaactgaaggcgggaaacgacaatct

SEQ ID NO: 18 - DNA, 300 bp, Artificial Sequence
  Nucleotide sequence of a Safflower scaffold sequence into which the T-DNA of GOR73240 has inserted (gx_S317_Scaff097804)
taagaaatggcggaacaaaattacaccgttattccggcgtccgagcatagaagaaaagcggatatttggcaatgttacgacttgtgcaagtttcccgatggtagcaaaaaagtgcgatgcaatcggtgtggcgactttctaaaacccgaatcgaattcgacgttaaagcgacattacgaacatagttgcaaggcgttgaaagcggtcgaagatccaaatcaaccaatacttgaccattccggtcaagtatggtcgtatgataacgatcttgcgcaacaaatgacgatgaaatgtgtaatt

SEQ ID NO: 19 - DNA, 175 bp, Artificial Sequence
  Nucleotide sequence of the GOR73240 Left border junction sequence (E40_LB_junction)
aagaaatatttgctagtgatgtacggttaaaaccaccccagtactttctaaaacccgaatcgaattcgacgttaaagcggtcgaagatccaaatcaaccaatacttgaccattccggtcaagtatggtcgtatgataacgatcttgcgcaacaaatgacgatgaaatgtgtaatt

SEQ ID NO: 20 - DNA, 44 bp, Artificial Sequence
  Nucleotide sequence of the Left border of the T-DNA in GOR73240 (pCW732_LB)
aagaaatatttgctagtgatgtacggttaaaaccaccccagtac

SEQ ID NO: 21 - DNA, 40 bp, Artificial Sequence
  Nucleotide sequence of a portion of the GOR73240 Right border junction
tcgaattcgacgttaaagcgacactgatagtttaaactga

SEQ ID NO: 22 - DNA, 40 bp, Artificial Sequence
  Nucleotide sequence of a portion of the GOR73240 Left border junction
ggttaaaaccaccccagtactttctaaaacccgaatcgaa

SEQ ID NO: 23 - DNA, 20 bp, Artificial Sequence
  Oligonucleotide primer
gtcggcaaatcgtcagactt

SEQ ID NO: 24 - DNA, 20 bp, Artificial Sequence
  Oligonucleotide primer
gtggagtcaggcttgatcgt

SEQ ID NO: 25 - DNA, 20 bp, Artificial Sequence
  Oligonucleotide primer
tgttcccggatcgaagtaag

SEQ ID NO: 26 - DNA, 20 bp, Artificial Sequence
  Oligonucleotide primer
acatccttggcgtctcaact

SEQ ID NO: 27 - DNA, 20 bp, Artificial Sequence
  Oligonucleotide primer
aatgttcgaatgcccttctc

SEQ ID NO: 28 - DNA, 18 bp, Artificial Sequence
  Oligonucleotide primer
agggcgacctctttttgg

SEQ ID NO: 29 - DNA, 20 bp, Artificial Sequence
  Oligonucleotide primer
gacaagtggtatgacattgc

SEQ ID NO: 30 - DNA, 18 bp, Artificial Sequence
  Oligonucleotide primer
ctaaaacaattcatccag

SEQ ID NO: 31 - DNA, 20 bp, Artificial Sequence
  Oligonucleotide primer
agatcctcttccgcttcctc

SEQ ID NO: 32 - DNA, 20 bp, Artificial Sequence
  Oligonucleotide primer
taccgggttggactcaagac

SEQ ID NO: 33 - DNA, 11060 bp, Artificial Sequence
  Nucleotide sequence of the insertion of the T-DNA in GOR73226 including the flanking safflower genomic sequences
caaaacatcttcgaatcgaaatcaaatttctaaaacgaattaaacaacaatggcgattatagctgatatctggaaacgaagatcaaaaacgaaaaagaaatcaacaaatctgaagaaacacgcactgtcacttacttgtaggtgtgagatatggctctgtctgtatgatgatcctctgcagatgtttgatagttcgagtttaatggagcaaattggctatggcgatttctaggtttacaagaattccttacttttctacaattcctcaaaactgacatctctgttttcctgcaaaaagctttcgtctcgaaaatggcggaagattttgttaaacaaatgctcccctcatttcgtctttcgaaagcgacaactttatagaggttttaaagtgaccgaatagtttcattgaaccccctcaaggtttaagatttggcaggaaaggccctttcttagtttcattcatatcctcttaaaatgcattcaaattttgtcgacggatgtttgagttttcttacaattttctttttgttaatatggggtttcgagtcggtttacttgcaccgactaacacctggatgaccataattgagatggagtcattgtatatgaacctttatgactgtaagggctttagtagcgtctgaatttgaatatggataaatgagattttccattacatgcattctgtaaagcttgaccaatgcacaacaatccaagtggtttatatttttagtatataacctaatttaaactgtttgatagtattgacttattaagtatggagtagatagatcattagatgtggattttagtttgggagcttgagattttagtgggttttaatcaaaactacatagttttgatgtgtgttgtacctaagcttaggtacataacagcaacacactccctttccccgctatatacatatatatagggttaggttattttgttaacccattaatttgtgttaaccgtgttaaccatataaccaccgttattttaaacactgatagtttaaactgaaggcgggaaacgacaatctgctagtggatctcccagtcacgacgttgtaaaacgggcgccccgcggaaagcttgctagccaattggggcccgcgttgggaattcgattgagctctaatacgactcactataggcggccgctctagactcaagcatacggacaagggtaaataacatagtcaccagaacataataaacaaaaagtgcagaagcaagactaaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaattctcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcagacccatctgtgccactcgtacaattacgagagttgttttttttgtgattttcctagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccaccatggatccctgcagtaaatcccgggctcgagacaagtttgtacaaaaaagcaggctccgcggccgcccatcacccaagagtcccgcagtctttacatgattaagggcagtttcctgcaaatgattcattagcgtctctatcgacgcagttcgatcagcccctatttcataggatctaatagagaagttttgacggaatacaagcccatcctgcacaattctccctaaaccaaaaggatccagatcagcaagcatgtcaggccgcttggttttccaatccagcatcatccattgtttctctgcagccaagaagattgtggtgattgcagcaagaagcatgctccaatccggcaactggttgataaaggttcttgggacatgtgacgtagaattatcgtctgtcttgatcatgggccgcactctagtcccatttacctctgcaggagcctgtgcagccttaacttgcaaaccgccagaagcctgcaggtaccaatggctcgacgacactgtcggccttatcctacattctgttcttcttgttccttacttctcgtggaaatacagccaccgccgccaccactccaacaccggctcgattgagcacgacgaggtcttcgtcccgaaactcaaatccggggtccggtccaccgcaaaatacctaaacaacccccccggccggatcttgacccttcttgtcaccctcaccctcggctggccgttgtacctcatgttcaacgtctccggccgctactacgaccgctttgcctgccattttgacccaaacagccccatctactcgaaccgtgaacgtgcccagatcttcatctccgacgccgggatcttcgccgtcctctacgggctctaccggctcgcggcggtcaaagggctcgtgtgggtcctgaccgtctatgccggaccgttgctcgtggtcaacgggtttcttgtcctgatcacgttcctccagcacacccacccgtcgttgccgcactacgactcaaccgagtgggactggctccgtggggccctggccaccatcgaccgtgactacgggattttaaacaaggtgttccacaacatcaccgacacccacgtgacccaccatctgttttcgacgatgccgcattatcacgcgatggaggccacgaaggcgataattccgatcctcggggattattatcagttcgatgggacgtcagtttttaaggcgatgtatagggagacgaaggagtgcatttatgtggataagggtgggcgcgccgacccagctttcttgtacaaagtggtctcgatcgaggaattcggtaccccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattatttttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaagctgggttcgaaatcgataagcttgcgctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcacaagcttggatcctctagaccactttgtacaagaaagctgggtcggcgcgcccacccttatccacataaatgcactccttcgtctccctatacatcgccttaaaaactgacgtcccatcgaactgataataatccccgaggatcggaattatcgccttcgtggcctccatcgcgtgataatgcggcatcgtcgaaaacagatggtgggtcacgtgggtgtcggtgatgttgtggaacaccttgtttaaaatcccgtagtcacggtcgatggtggccagggccccacggagccagtcccactcggttgagtcgtagtgcggcaacgacgggtgggtgtgctggaggaacgtgatcaggacaagaaacccgttgaccacgagcaacggtccggcatagacggtcaggacccacacgagccctttgaccgccgcgagccggtagagcccgtagaggacggcgaagatcccggcgtcggagatgaagatctgggcacgttcacggttcgagtagatggggctgtttgggtcaaaatggcaggcaaagcggtcgtagtagcggccggagacgttgaacatgaggtacaacggccagccgagggtgagggtgacaagaagggtcaagatccggccgggggggttgtttaggtattttgcggtggaccggaccccggatttgagtttcgggacgaagacctcgtcgtgctcaatcgagccggtgttggagtggtggcggcggtggctgtatttccacgagaagtaaggaacaagaagaacagaatgtaggataaggccgacagtgtcgtcgagccattggtacctgcaggcttctggcggtttgcaagttaaggctgcacaggctcctgcagaggtaaatgggactagagtgcggcccatgatcaagacagacgataattctacgtcacatgtcccaagaacctttatcaaccagttgccggattggagcatgcttcttgctgcaatcaccacaatcttcttggctgcagagaaacaatggatgatgctggattggaaaaccaagcggcctgacatgcttgctgatctggatccttttggtttagggagaattgtgcaggatgggcttgtattccgtcaaaacttctctattagatcctatgaaataggggctgatcgaactgcgtcgatagagacgctaatgaatcatttgcaggaaactgcccttaatcatgtaaagactgcgggactcttgggtgatgggcggccgcggagcctgcttttttgtacaaacttgtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagggcccgacgtcgcatgctcctctagactcgacgcgtcctagagatccgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgaccccgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccggagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcatcgatgatatcagatccgggcggaaataggtaaagaagttgcggataaggtaattgccattgcagattatttggattgagagtgaatatgagactctaattggataccgaggggaatttatggaacgtcagtggagcatttttgacaagaaatatttgctagtgatgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagagcgatcgcagctttctgagccgccgattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcatgggttaacaaaataactctcctctatatatatatatatatattgaacgtcaagttaaacacacacacgctccagcggtatcgggcctccgattgggcaatcccactaatccgttgcgacctggcccaacctatggctgaacccaaccagctgggaagtggatcccatagactggatcctcccataccggtcccgaacccggcttcgaaaaaccagatcctttattgaccggatgagtggccaagatttgtcctcatgttcacaaccaaaattgagttcacatttgaacttcaaatgtgaacccaatttttgttgtgaacgtgagaacgaatctcagccttcggttatggtagacgcgatcacggaggattgacgcgattcatgggagggtggagatttaaaggaggggacccggtcaaagccgtctttattgaccggtgagtggctgagattcgtcctcatgttttcttaaaaggttatgtttctcgtatgaaactttctctctctatatatatatattgaatgatttacaattatatatttttaacatattaaacgaactttaatatattccttcatatgtgaattcaaacttaaaagtaaaggttatgtaaactttttccatattccaaattcaaataacacaagaaatttaaaattctatgtcagggactttatatgacaaaaagaaaaagtgaaagggagatttttcaaaatttgaaatcattacagcgcgcagggcgcagcttctgaaaggtggaatcatgtaaatataattggggaaaatggggaaaaaagtaaggcaattttttaaaatctcaaaaaataaacaaaattgtggaaggatgattgaaaaaaatatttctcaaatattataatttaccataatatatgtttttttcctattttaaacatttttttatttaggccgtgcgtagcacaatagcggcttcgctgcggtttggccgtgggctcggcatgcttagcctgcgcaagctac

SEQ ID NO: 34 - DNA, 10726 bp, Artificial Sequence
  Nucleotide sequence of the insertion of the T-DNA in GOR73240 including the flanking safflower genomic sequences
tctcgaatatcggcaaaacatatgcccataaatgacttaaaaaataaaagtacccctccccccctctccctccctcttctccttttagtccaaaaagcagcaaaatataaagattgaatagaaaaaagcatacaaaaaatatcaatttattagaaaatgtgttaataatttgattgagattaagaaaatgttgagatttatataattataaaataaactttgggttctcctttctttgtaccatctttgttcttttttaataaaattgttgacctttcaaacaaaaaatatttcccatttatgtttcatatttaacctttttatttggatgtagtcttacttacttaggattagtgatggtcaaacgaagccgaaccgaaccgatcgaaaaaaccaaacccgaaccgacccgaaccggtttttggcccgaactgatttgaaccggttttgggcctgcgggcctgaaccggtcccgggtattatagaattcttcggtccaatcccggtttgaacccgaaccggtttaacggtcacttggaaaattttcaaaaattctagccgttaaggagccgttggactagccgtttttttggtttttttgtggtttttttgcttttattacaccttcaaatgcatataaatagtaggggaaggttatggctcaaaccaccaattcccgaatagtaaaaaacgcaaaaaaacgcaaagatttcttatttttcggctcccgtaatctcgtattctctaatctcgattctcgagttctcgtgttctcgtattctctaatctcgagttctcgtgttctcgtattctctaatctcgagttctcgtgttctcgtgttctcgtgttctcgtattttctaatttctattttctagttatctcgtattaagaaatggcggaacaaaattacaccgttattccggcgtccgagcatagaagaaaagcggatatttggcaatgttacgacttgtgcaagtttcccgatggtagcaaaaaagtgcgatgcaatcggtgtggcgactttctaaaacccgaatcgaattcgacgttaaagcgacactgatagtttaaactgaaggcgggaaacgacaatctgctagtggatctcccagtcacgacgttgtaaaacgggcgccccgcggaaagcttgctagccaattggggcccgcgttgggaattcgattgagctctaatacgactcactataggcggccgctctagactcaagcatacggacaagggtaaataacatagtcaccagaacataataaacaaaaagtgcagaagcaagactaaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaattctcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcagacccatctgtgccactcgtacaattacgagagttgttttttttgtgattttcctagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccaccatggatccctgcagtaaatcccgggctcgagacaagtttgtacaaaaaagcaggctccgcggccgcccatcacccaagagtcccgcagtctttacatgattaagggcagtttcctgcaaatgattcattagcgtctctatcgacgcagttcgatcagcccctatttcataggatctaatagagaagttttgacggaatacaagcccatcctgcacaattctccctaaaccaaaaggatccagatcagcaagcatgtcaggccgcttggttttccaatccagcatcatccattgtttctctgcagccaagaagattgtggtgattgcagcaagaagcatgctccaatccggcaactggttgataaaggttcttgggacatgtgacgtagaattatcgtctgtcttgatcatgggccgcactctagtcccatttacctctgcaggagcctgtgcagccttaacttgcaaaccgccagaagcctgcaggtaccaatggctcgacgacactgtcggccttatcctacattctgttcttcttgttccttacttctcgtggaaatacagccaccgccgccaccactccaacaccggctcgattgagcacgacgaggtcttcgtcccgaaactcaaatccggggtccggtccaccgcaaaatacctaaacaacccccccggccggatcttgacccttcttgtcaccctcaccctcggctggccgttgtacctcatgttcaacgtctccggccgctactacgaccgctttgcctgccattttgacccaaacagccccatctactcgaaccgtgaacgtgcccagatcttcatctccgacgccgggatcttcgccgtcctctacgggctctaccggctcgcggcggtcaaagggctcgtgtgggtcctgaccgtctatgccggaccgttgctcgtggtcaacgggtttcttgtcctgatcacgttcctccagcacacccacccgtcgttgccgcactacgactcaaccgagtgggactggctccgtggggccctggccaccatcgaccgtgactacgggattttaaacaaggtgttccacaacatcaccgacacccacgtgacccaccatctgttttcgacgatgccgcattatcacgcgatggaggccacgaaggcgataattccgatcctcggggattattatcagttcgatgggacgtcagtttttaaggcgatgtatagggagacgaaggagtgcatttatgtggataagggtgggcgcgccgacccagctttcttgtacaaagtggtctcgatcgaggaattcggtaccccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattatttttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaagctgggttcgaaatcgataagcttgcgctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcacaagcttggatcctctagaccactttgtacaagaaagctgggtcggcgcgcccacccttatccacataaatgcactccttcgtctccctatacatcgccttaaaaactgacgtcccatcgaactgataataatccccgaggatcggaattatcgccttcgtggcctccatcgcgtgataatgcggcatcgtcgaaaacagatggtgggtcacgtgggtgtcggtgatgttgtggaacaccttgtttaaaatcccgtagtcacggtcgatggtggccagggccccacggagccagtcccactcggttgagtcgtagtgcggcaacgacgggtgggtgtgctggaggaacgtgatcaggacaagaaacccgttgaccacgagcaacggtccggcatagacggtcaggacccacacgagccctttgaccgccgcgagccggtagagcccgtagaggacggcgaagatcccggcgtcggagatgaagatctgggcacgttcacggttcgagtagatggggctgtttgggtcaaaatggcaggcaaagcggtcgtagtagcggccggagacgttgaacatgaggtacaacggccagccgagggtgagggtgacaagaagggtcaagatccggccgggggggttgtttaggtattttgcggtggaccggaccccggatttgagtttcgggacgaagacctcgtcgtgctcaatcgagccggtgttggagtggtggcggcggtggctgtatttccacgagaagtaaggaacaagaagaacagaatgtaggataaggccgacagtgtcgtcgagccattggtacctgcaggcttctggcggtttgcaagttaaggctgcacaggctcctgcagaggtaaatgggactagagtgcggcccatgatcaagacagacgataattctacgtcacatgtcccaagaacctttatcaaccagttgccggattggagcatgcttcttgctgcaatcaccacaatcttcttggctgcagagaaacaatggatgatgctggattggaaaaccaagcggcctgacatgcttgctgatctggatccttttggtttagggagaattgtgcaggatgggcttgtattccgtcaaaacttctctattagatcctatgaaataggggctgatcgaactgcgtcgatagagacgctaatgaatcatttgcaggaaactgcccttaatcatgtaaagactgcgggactcttgggtgatgggcggccgcggagcctgcttttttgtacaaacttgtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagggcccgacgtcgcatgctcctctagactcgacgcgtcctagagatccgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgaccccgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccggagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcatcgatgatatcagatccgggcggaaataggtaaagaagttgcggataaggtaattgccattgcagattatttggattgagagtgaatatgagactctaattggataccgaggggaatttatggaacgtcagtggagcatttttgacaagaaatatttgctagtgatgtacggttaaaaccaccccagtactttctaaaacccgaatcgaattcgacgttaaagcggtcgaagatccaaatcaaccaatacttgaccattccggtcaagtatggtcgtatgataacgatcttgcgcaacaaatgacgatgaaatgtgtaattcaaatggcgttgtcgtttggacatttcgataacacgcacattacgaacttgattcgtaaatacttccaaccacgctacgaaccggtaagtcgtcaaactcttagacgcgatgctataagatattggctcaaagctaaaaaagtacgaaataaatgttttcaaaattaccctcgagggtttctttaaccgccgatatatggagcgcaccaaacggattgccacaatcctatttatgcataacggcacattggattatgccggaagcgtggcaattatgcaagcgtgtaatcgcgtttgaacactttggttcgccacattcggagaacgtcaatattctataattatgagaattcttaaaatatggtttaaaaactagtattattagtatgagttttgataacgctagtaacaataccgcatgtattgatagattacgtttggttttatcccctattttagatggaaaattgtttcacacattatgtgtcgcacacataataaacttatccgttcaagacggcctaagagtatttgatcccgttttaactaagtttaggcttttgatacgtcggatttttgcaaaaggaaaaagatatcaaaacttatttatagacttttgcactagtgtcgattttaaagctttgtcgggggctcacgatatgccggttcgatggaactcgacttggaaaatgttggataatttaataagacaaagacaatatattcaaccattttataatctccatttcccgcataacgcaataactaatactgattgggaaatcattcaaagttttcacgatattttgttcgatttcaaaactgcaacaacg

SEQ ID NO: 35 - DNA, 27 bp, Artificial Sequence
  Oligonucleotide primer
ttggataccgaggggaatttatggaac

SEQ ID NO: 36 - DNA, 20 bp, Artificial Sequence
  Oligonucleotide primer
caatcacaataagtcgttgc

SEQ ID NO: 37 - DNA, 27 bp, Artificial Sequence
  Oligonucleotide primer
ttggataccgaggggaatttatggaac

SEQ ID NO: 38 - DNA, 20 bp, Artificial Sequence
  Oligonucleotide primer
tgataacgatcttgcgcaac

SEQ ID NO: 39 - DNA, 19 bp, Artificial Sequence
  Oligonucleotide primer
aaaagcctgaactcaccgc

SEQ ID NO: 40 - DNA, 19 bp, Artificial Sequence
  Oligonucleotide primer
tcgtccatcacagtttgcc